(19) United States
(12) Patent Application Publication
(10) Pub. No.: US 2005/0048589 A1
(43) Pub. Date: Mar. 3, 2005

(54) Lung cancer specific gene products: their coding sequence, their antibodies and their use in diagnostic, therapeutic and disease management of lung cancer

(76) Inventor: Moncef Jendoubi, San Francisco, CA (US)

Correspondence Address: ORRICK, HERRINGTON & SUTCLIFFE, LLP, 4 PARK PLAZA, SUITE 1600, IRVINE, CA, 92614-2558, US

(21) Appl. No.: 10/926,543

(22) Filed: Aug. 25, 2004

Related U.S. Application Data
(60) Provisional application No. 60/497,790, filed on Aug. 25, 2003.

(51) Int. Cl. G01N 33/574; C07K 16/30

(52) U.S. Cl. 435/7.23; 530/388.8

(57) ABSTRACT

The present invention relates to the disclosure of a group of gene products, polypeptides and their derivatives, their corresponding polynucleotide sequences (DNA and RNA), and specific antibodies to said gene products and derivatives. It is shown that said gene products are differentially expressed in lung cancer patients versus normal controls, using either tissues or biological fluids as specimens. Also provided is a procedure for producing the cancer specific gene products by recombinant techniques. This invention also pertains to the use of said antibodies in characterizing the gene products of the present invention, and in diagnostic, and disease management applications. The present invention also pertains to the therapeutic use of said antibodies and gene products to treat cancer and other human diseases. The invention also relates to composition, kits, and methods for detecting, characterizing, preventing, and treating human lung cancer.

### RELATED INFORMATION

This application claims priority over U.S. provisional application Ser. No. 60/497,790 filed on Aug. 25, 2003. The priority of the prior application is expressly claimed, and the disclosure of the prior application is hereby incorporated by reference in its entirety.

### FIELD OF THE INVENTION

The field of the invention is lung cancer. The invention includes disclosure of genes, gene-products and antibodies associated with lung cancer. The invention further includes composition and methods of use for diagnosis, disease management and therapy of lung cancer.

### BACKGROUND OF THE INVENTION

Cancer is the second leading cause of death in the US and a major health burden in industrialized countries. The National Institutes of Health estimated overall costs for cancer in the year 2002 at $171.6 billion: $60.9 billion for direct medical costs (total of all health expenditures), $15.5 billion for indirect morbidity costs (cost of lost productivity due to illness) and $95.2 billion for indirect mortality costs (cost of lost productivity due to premature death). The four major cancers affect breast, colon, lung and prostate.

Every type of cancer has been associated with chromosomal alterations, alterations in gene sequences and gene expression patterns, and modifications in protein levels, structure, or function. These molecular markers have potential use in early detection of cancer, in determining prognosis, monitoring disease progression, metastasis or therapeutic response.

However, of all the potential markers described in cancer, very few have actually reached the clinical setting. While screening for prostate-specific antigen (PSA) is now part of routine physical examination procedures of male population in the US, there is a need for more accurate prostate cancer screening markers. Indeed, PSA is more prostate specific than cancer specific and leads to high false positive rates. Another example of marker used in the clinic is carcinoembryonic antigen (CEA) to monitor disease progression and therapy response of colorectal cancer. However, only a proportion of colon cancer patients express elevated CEA levels at the time of diagnosis, and CEA may be elevated in other cancer types as well. Finally, imaging is being used to detect early breast cancers in population-wide screening, but this approach has become highly controversial. For most other types of cancer, including lung cancer, there are no early detection markers available.

In addition to diagnostics, another area in which accurate molecular markers are needed is cancer therapy to determine patient treatment response. Indeed, only 50-60% of patients, on average, responds to approved drugs, and most drugs have serious adverse side effects, particularly among cancer chemotherapies. There is a need to identify the subset of cancer patients that will successfully respond to a given cancer therapy. A compelling example is provided by a certain type of breast cancer cells expressing high levels of the tyrosine receptor kinase ERB2 (also known as HER2/neu) approximately representing 25% of breast cancer patients. Such cancer cells are more likely to respond to transzumab (Herceptin) therapy based on a therapeutic monoclonal antibody that specifically targets this receptor. Patients' tumor is first tested for ERB2 expression, before the drug is administered.

There is a need to develop novel, better and personalized therapeutic agents that are effective against various subtypes of cancer and patients' response. In conclusion: a) improved molecular markers and non-invasive early detection methods are needed to prevent the majority of cancers, b) more accurate (both sensitive and specific) markers are needed for cancers that are already associated to an early detection methods, and c) more reliable markers, especially serum markers, are needed for prognosis, metastasis and treatment response.

A fundamental issue in the biological sciences and in biotechnology is to investigate how an organism's genome regulates and maintains its functions in the normal state, and how alterations in genome products contribute to the disease state of an organism. To find new or better treatment against disease, or to develop new drugs, we need to understand the function of gene products, correlate their activities to disease, and unravel cellular pathways in the physiological and pathological states.

Genomic approaches capitalizing on DNA sequence information and bioinformatic analysis, are so-called “gene to function” approaches, whereby potential targets are relatively uncharacterized genes or gene products, that are selected on the basis of sequence motifs homologous to molecules known to be part of certain cellular pathways of relevance to the disease under examination. These potential targets are then further investigated in order to be assigned a biological function.

Over the past 10 years much effort has been devoted to the comparative analysis of gene expression profiles in different tissues, developmental stages, or in specific physiological and pathological conditions, using methods such as subtractive hybridization, differential display, serial analysis of gene expression (SAGE). Recent years of target discovery have been dominated by DNA microarrays, particularly in relation to cancer. However, mRNA expression patterns do not provide the most accurate molecular signature of a disease state. Indeed, mRNA expression levels are an indirect measure of protein expression, as mRNA levels do not always correlate with protein levels. There is also increasing evidence that most of the RNA transcribed from the genome is not translated into proteins, and that there are non-protein coding DNA sequences and RNA species in the genome, such as interfering RNA and microRNAs. Finally, the relatively limited set of genes in the eukaryotic genome encodes a highly complex proteome via a variety of mechanisms of regulation and expression strategies, including RNA splicing and post-translational modifications and cleavage.

Proteins are the end product of gene expression, and the major players in cellular processes. It is the concerted action of gene products, in time and space, within the complex network of cellular interactions that eventually results in a given phenotype, including a disease status. Proteins are therefore the major targets for diagnostic and therapeutic interventions and the ultimate target for the pharmaceutical industry. There is a need to discover and identify diagnostic markers and therapeutic targets through proteomic investigations.

Lung cancer is the leading cause of cancer death in both men and women in the United States accounting for an estimated 157,200 deaths in 2003 and 25% of all cancer deaths (Jemal, 2002). Cigarette smoking is by far the most important risk factor in the development of lung cancer accounting for over 80% of all lung cancers. Other risk factors include occupational, medical and environmental exposure to certain industrial substances (ACS, 2003). Since 1990, death rates have significantly declined in men, while rates for women have continued to increase, although at a much slower pace. However, since 1987, more women have died of lung cancer than of breast cancer. Such decreased lung cancer incidence and mortality rates most likely result from cigarette smoking cessation programs. Decreasing smoking patterns among women lag behind those of men thus explaining these figures. One of the reasons for the deadly toll of this disease, is that only 15% of lung cancers are diagnosed at an early stage. Resection is an option only for patients presenting with localized disease (i.e. at an early stage), in which case the survival rate is 48%. When the tumor has spread to lymph nodes, the 5-year survival is reduced to 5-13% (ACS, 2003).

Lung cancer treatment includes surgery, if disease in not too advanced, radiation therapy and chemotherapy. Overall, the prospects of lung cancer patients remain limited at present and new therapeutic strategies are eagerly awaited.

There are four major histologic types of lung cancer (Carbone, 1997), including small cell carcinoma (SCLC) and the three non-small cell lung carcinoma (NSCLC), which altogether account for approximately 75% of all lung cancer cases. This distinction is clinically important due to their different response to therapy, as SCLC is more responsive to chemotherapy than NSCLC. Among NSCLC: adenocarcinoma are peripheral and represent 30-40% of lung cancers, while squamous cell carcinoma and large cell carcinoma represent 20-25% and 15-20% of lung cancers, respectively. Adenocarcinoma are further classified into three subgroups, including bronchioloalveolar carcinoma (BAC; Brambilla, 2001).

Early detection screening by chest radiography and sputum cytology are not sufficiently sensitive, so the American Cancer Society (ACS) has not recommended screening for early lung cancer detection, even in individuals at high risk for lung cancer (ACS, 2003). New imaging technologies such as low dose spiral multi-slice helical computed tomography (CT) offers promise in detecting early lung cancers compared with the chest X-ray, as it detects much small peripheral tumors. However, a CT drawback is the high rate of incidental nodule detection, and the subsequent morbidity cost associated with biopsy of these nodules.

There are many chromosomal abnormalities such as loss of heterozygosity (LOH) alterations, as well as oncogenes, tumor suppressor genes and other cell cycle related genes that have been identified and associated to the pathogenesis of lung cancer (Minna, 2003). These molecular determinants may be potential candidates for early diagnostic markers or prognostic indicators of disease stage, progression and survival. However, no single marker has so far met sufficient specificity and sensitivity to be recognized of clinically significant value.

In several malignancies including ovarian, colorectal and breast cancers, serum tumor markers have been successful in evaluating prognosis or response to therapy. Serum markers such as carcinoembryonic antigen (CEA) and CA-125, serum cytokeratin fragment 21-1 (CYFRA 21-1) and the extracellular soluble fraction of the c-erb-2 protein (a member of the EGF receptor protein family) are found to be elevated in lung cancers. However, none is useful for early lung cancer detection.

Thus, lung cancer remains a deadly disease with no clinically or diagnostically useful early detection marker. Unfortunately, smoking trends among youth have increased considerably in the US, and smoking in adult population is still high worldwide. Further, lung cancer risk persists even fifteen years after smoking cessation, and the cancer itself may take over ten years before becoming clinically evident (Witsuba, 1997). This latency period assures that we will witness a high rate of lung cancer for decades ahead. For this and the above reasons, lung cancer remains a national and global public health priority.

In summary, there is a tremendous need for lung cancer early diagnostic markers and for the development of reliable, accurate and non invasive diagnostic tests enabling early detection as well as prognostic evaluation, using biological specimens such as serum. Therefore, the identification and characterization of novel and improved biomarkers for lung cancer is of paramount importance, especially for early diagnosis, staging, prognosis, treatment response, metastasis indication, surveillance and disease management.

### SUMMARY OF THE INVENTION

The present invention discloses gene products associated to lung cancer, their DNA (RNA) sequences, their polynucleotides and peptide derivatives, and antibodies having specific binding affinity to the protein production expressed by the identified genes. The present invention also relates to the use of the above mentioned molecules in diagnostic and therapeutic applications.

The present invention includes a number of polypeptide sequences of human origin, as well as fragments, analogs and derivatives thereof, and a number of nucleotide sequences (DNA or RNA) of human origin encoding in all or in part, the identified polypeptides.

The invention also includes a process for producing those polypeptides by recombinant techniques, including expression in bacterial and mammalian systems, and purification of the relevant gene products. The present invention includes methods for the production of polyclonal antibodies, as well as of monospecific antibodies against the relevant gene products. Such antibodies have a wide variety of utilities, many of which are exemplified in the teachings herein.

The present invention identifies differential expression of specific gene products in normal and diseased tissues as well as in biological fluids, derived from normal individuals and lung cancer patients, by means of immunodetection using the antibodies mentioned above. Likewise, the present invention includes the use of the above mentioned antibodies to assess the relative expression of the relevant gene products in different histological types of lung cancer. Hence, the present invention provides evidence for the specific overexpression or underexpression of those gene products in relation to lung cancer as compared to normal. The present invention further provides evidence for the function of one or all such gene products as markers for lung cancer detection, progression and disease management, whether those gene products are present in tissues, or secreted in biological fluids.

The identified antibodies having specific binding affinity for lung cancer related gene products, also referred to as targets or markers, are useful for in vivo and in vitro diagnostic applications. The diagnostic method can utilize one or a panel of antibodies that recognize lung cancer specific targets, whether the antibodies are specific for the organ site, the histological type or subtype of the tumor, or even the stage of disease. The present invention further provides a method to identify, and thus diagnose, diseased individuals within a population of unknown samples. In an extension of the above embodiment, the present invention thus further provides the composition of a diagnostic kit for detection and disease management of lung cancer.

The antibodies of the present invention have further utility in the characterization of lung cancer related gene products, as provided in further embodiments of the present invention, including but not restricted to the determination of their apparent molecular weight, their cellular localization (e.g. to the nucleus, the cytoplasm or the cell membrane), their presence in fixed tissue microarrays, and the purification of the target gene products through biochemical techniques known to those skilled in the art.

The present invention further provides the ability to use the antibodies in a number of applications such as, but not restricted to analysis of the biological activity and function of the lung-cancer-specific gene products in relation to cellular pathways and networks in normal and disease state, investigation of toxicity profiles, expression of lung cancer associated gene products in normal tissues or in animal models of cancer to determine the therapeutic potential of said lung cancer specific targets. The present invention further discusses the utility of the lung cancer specific antibodies in therapeutic applications for the treatment of lung cancer.

The present invention takes advantage of: (a) the need to analyze gene expression at the protein level and in particular the tremendous need to discover specific and sensitive markers for lung cancer, as well as accurate, non-invasive and user-friendly diagnostic assays for lung cancer, as well known by those skilled in the art; (b) the need to develop novel therapeutic assays and molecules for the treatment of lung cancer and the management of that disease, as well known by those skilled in the art; (c) the exquisite specificity of interaction between two molecules, preferably an antibody, and its protein target even within a complex biological mixture; (d) a process namely a matrix protein array that enables to simultaneously assay a multiplicity of biological samples, in various physiological conditions; (e) the broad use of antibodies in multiple and diversified assays, including multiplex format for rapid high-throughput analysis of biological samples.

In conclusion, the present invention provides the composition for a multiplicity of polypeptides, polynucleotides, antibodies and derivatives, corresponding to lung cancer gene products. The present invention further provides certain characterization, biological activities and functions for the above mentioned molecules. The present invention further provides the utility of the above molecules in diagnostic and therapeutic applications. It is a unique aspect of the present invention to provide correlation between the relevant gene products and lung cancer by examination of the relative expression levels of proteins in a large number of human specimens. The present invention should greatly improve the diagnosis and treatment of most lung cancers.

The following Examples are provided to further illustrate the present invention and the procedure by which the molecules of the present invention may be prepared. The Examples are in no way restrictive, and numerous modifications and variations of the present invention are possible in light of the teachings herein, and therefore, within the scope of the appended claims, the invention may be practiced otherwise than as particularly described.

### BRIEF DESCRIPTION OF FIGURES AND TABLES

FIGS. 1A to 1D: Reactivity of selected antibodies by matrix protein array analysis of normal and diseased tissues from lung cancer patients (protein sets 1 to 5).

Differential protein expression between normal and diseased samples is shown in a matrix protein array experiment using selected antibodies of the present invention. Protein extracts prepared from matched normal and diseased lung tissues from lung cancer patients are printed, in the same amount, in each chamber of the matrix protein array. Each chamber displays the same matrix of samples. Then each chamber of the matrix protein array receives one single antibody. Antibodies are tested at the dilution of 1/2000. Immunodetection is by chemiluminescence reaction. Detailed description of matrix protein array analysis can be found in Example 2, 3 and 4. The schematic diagram below each figure provides in a table format (rows and columns) the ID No of the antibodies used in the experiment, and their location in the matrix protein array.

Protein samples used in experiment 1A are referred to as “protein set 1”, where protein samples are prepared from matched normal and cancer specimens from bronchioalveolar lung carcinoma. Protein samples used in experiment 1B are referred to as “protein set 2”, where protein samples are prepared from matched normal and cancer specimens from lung adenocarcinoma. Protein samples used in experiment 1C are referred to as “protein set 3 and 4”, where protein samples are prepared from matched normal and cancer specimens from squamous cell lung carcinoma. Protein samples used in experiment ID are referred to as “protein set 5”, where protein samples are prepared from matched normal and cancer specimens from lung adenocarcinoma, and printed in duplicate. Table 1A provides detailed clinical information on the protein sets used, such as the lung cancer tissue type, and identifies lung specimens by an ID tag. Table1B provides the matrix location of the protein samples used.

FIG. 2: Reactivity of selected antibodies by matrix protein array analysis of plasma from normal and lung cancer patients (protein set 6).

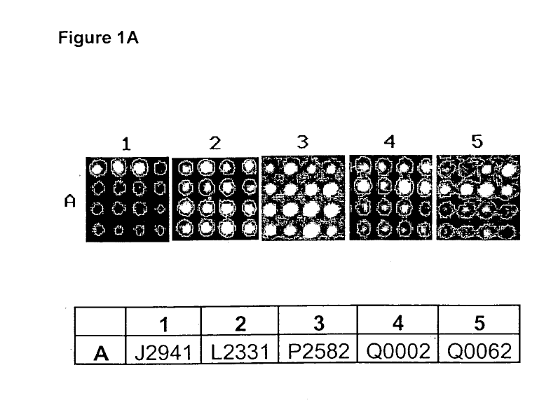

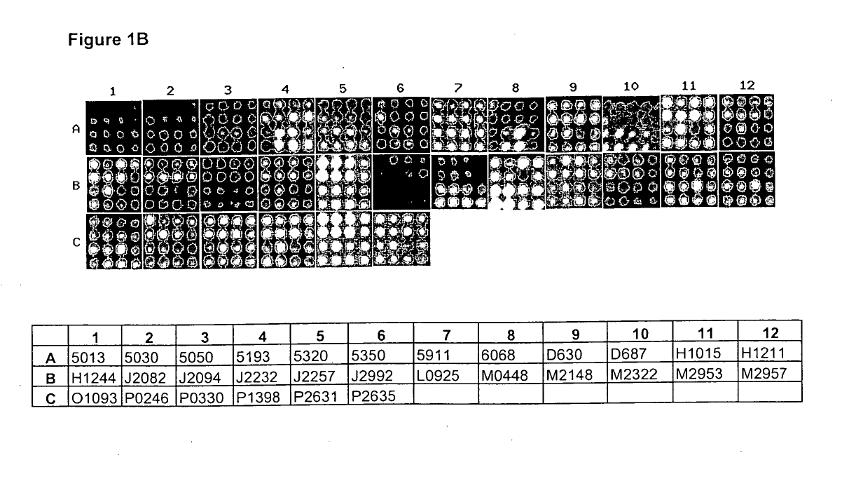

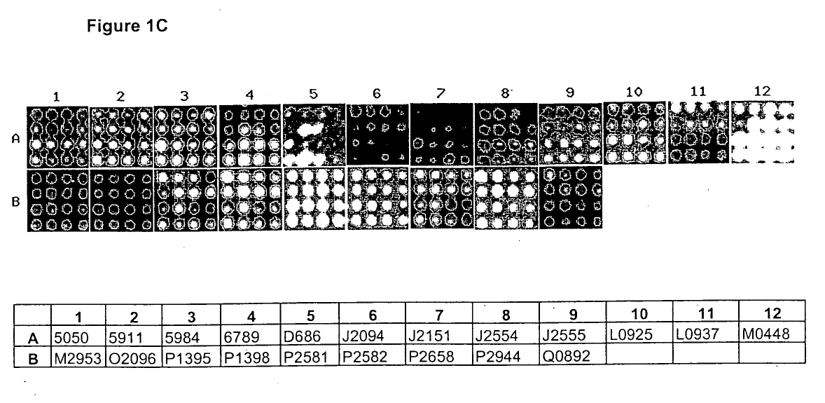

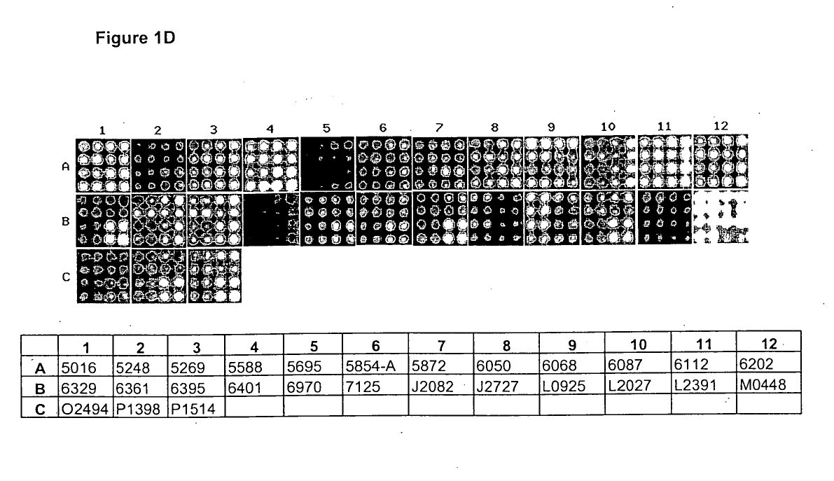

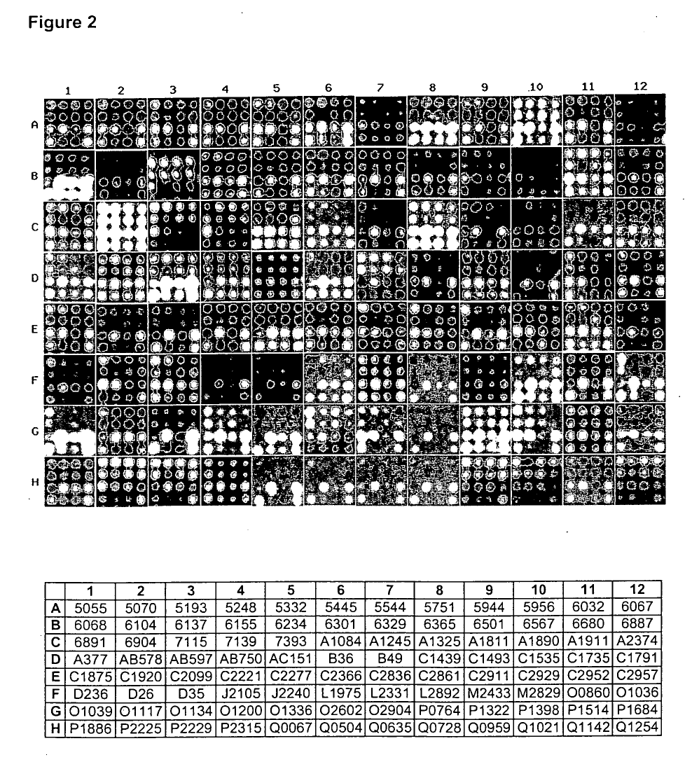

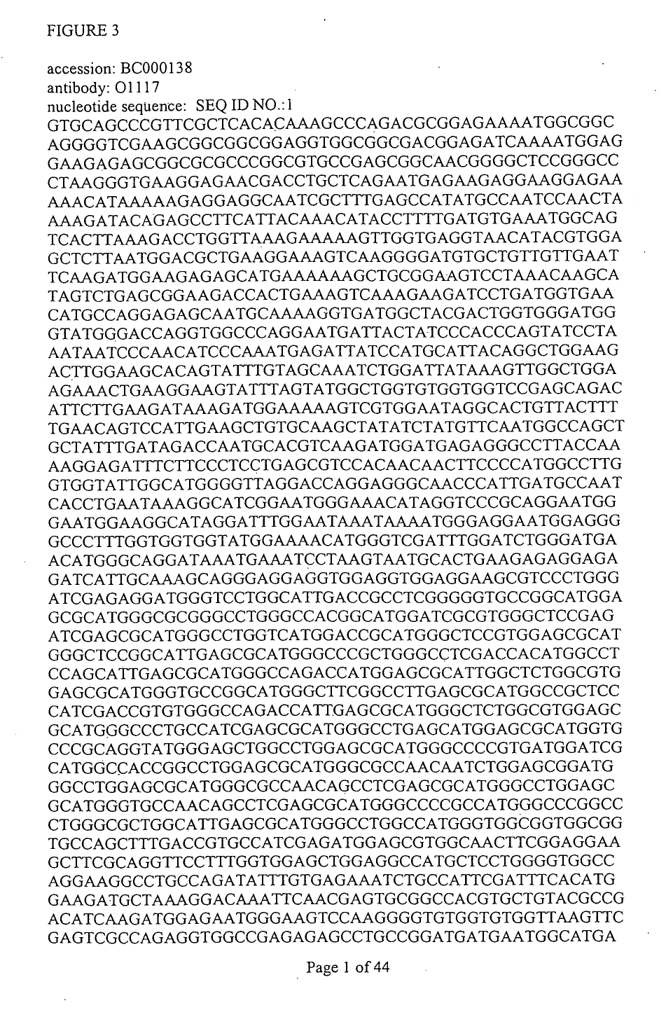

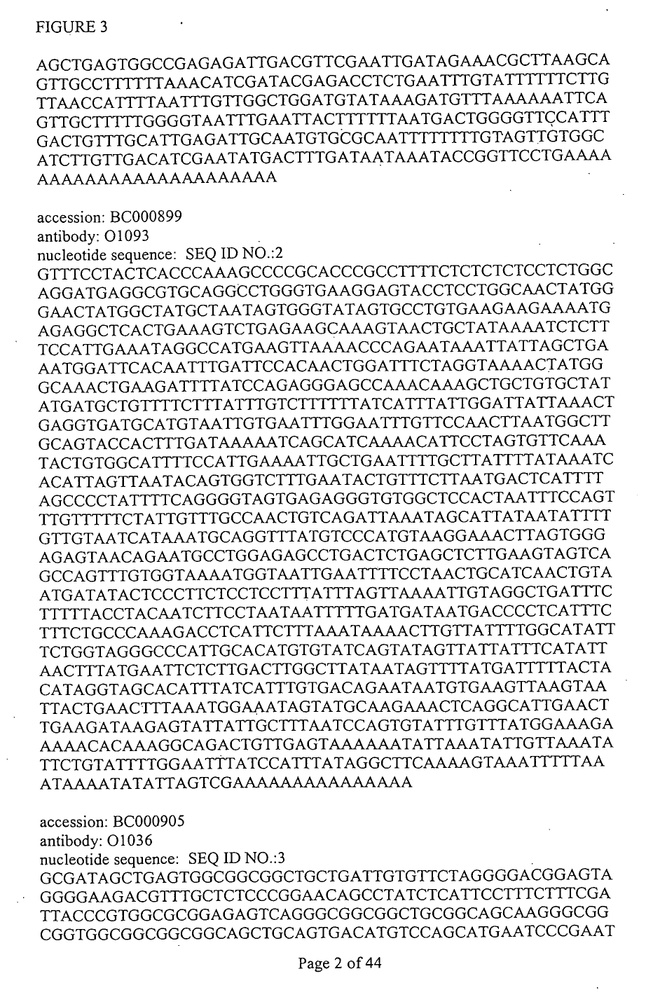

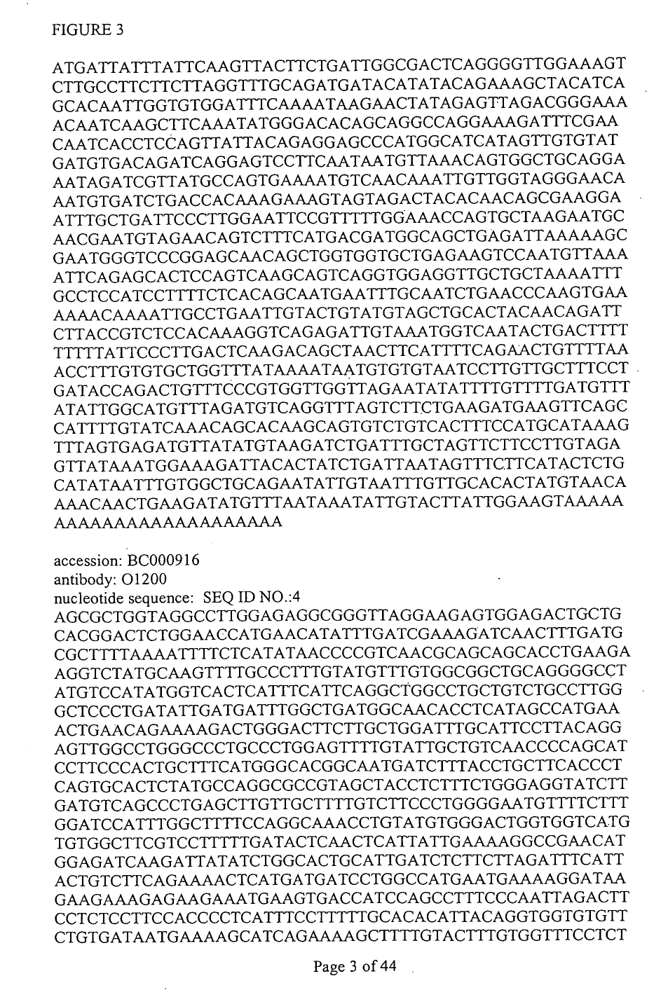

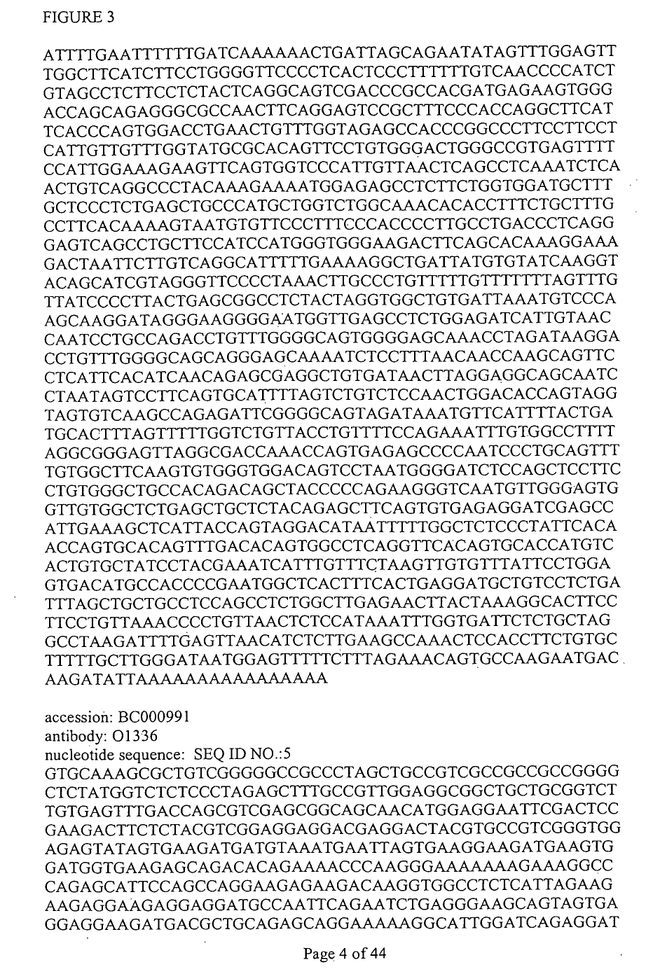

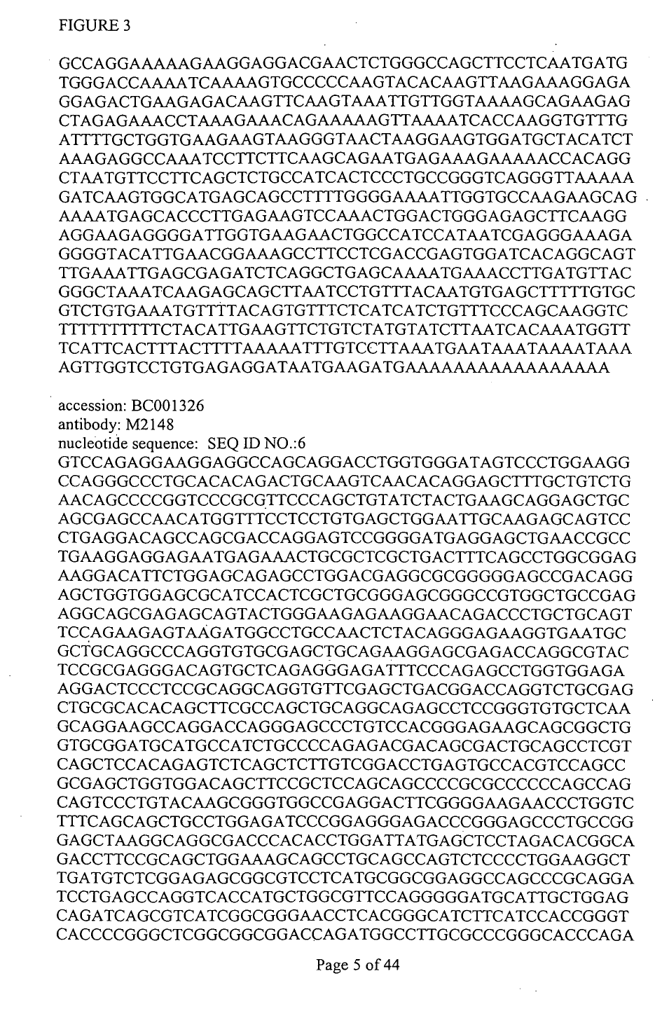

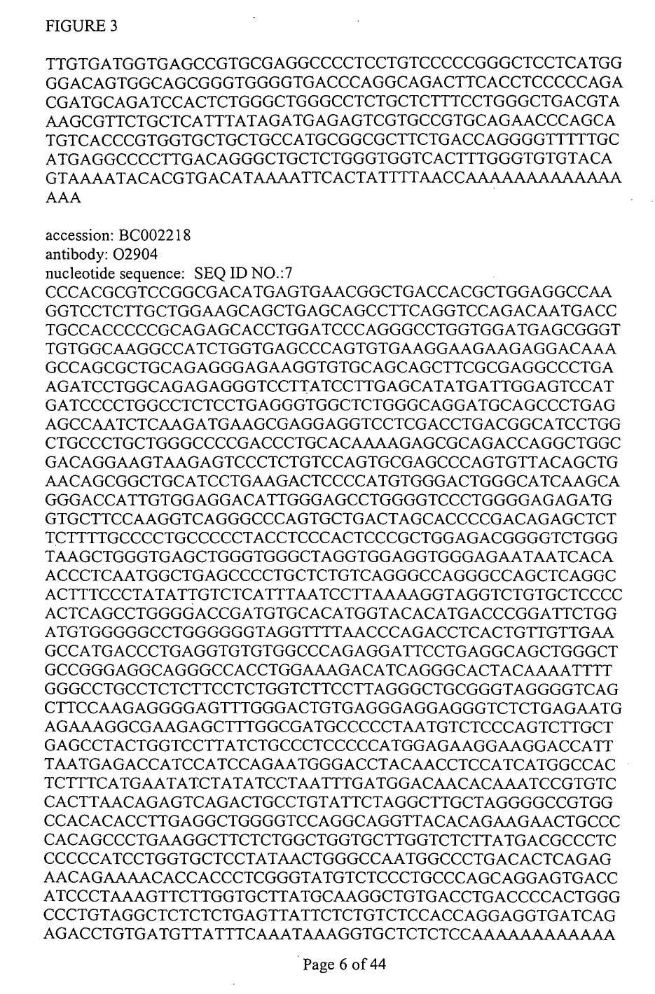

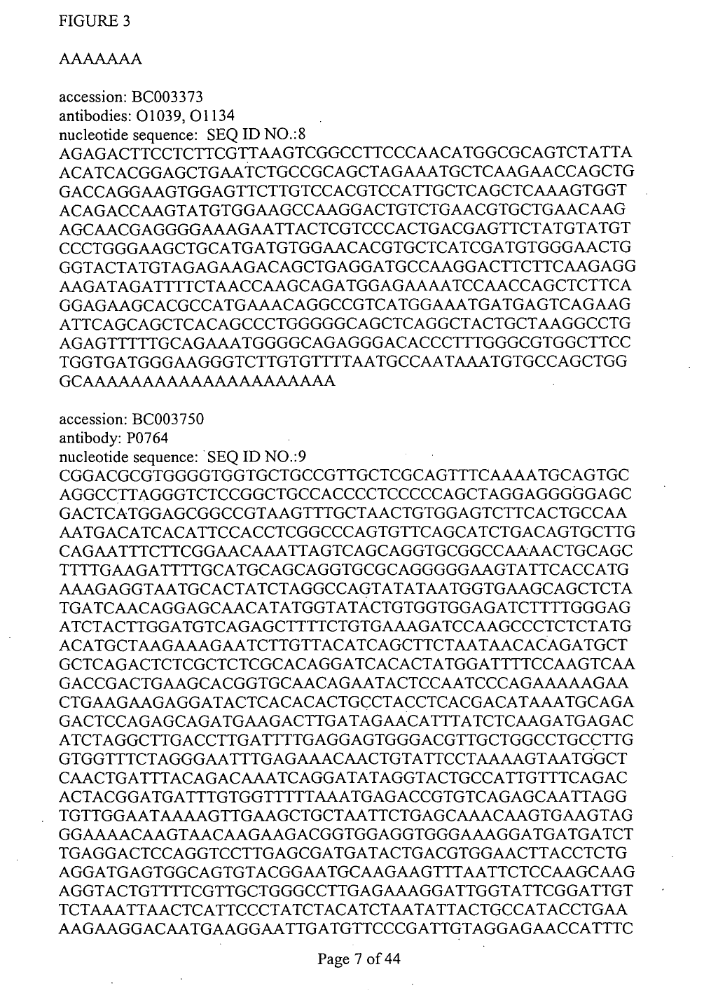

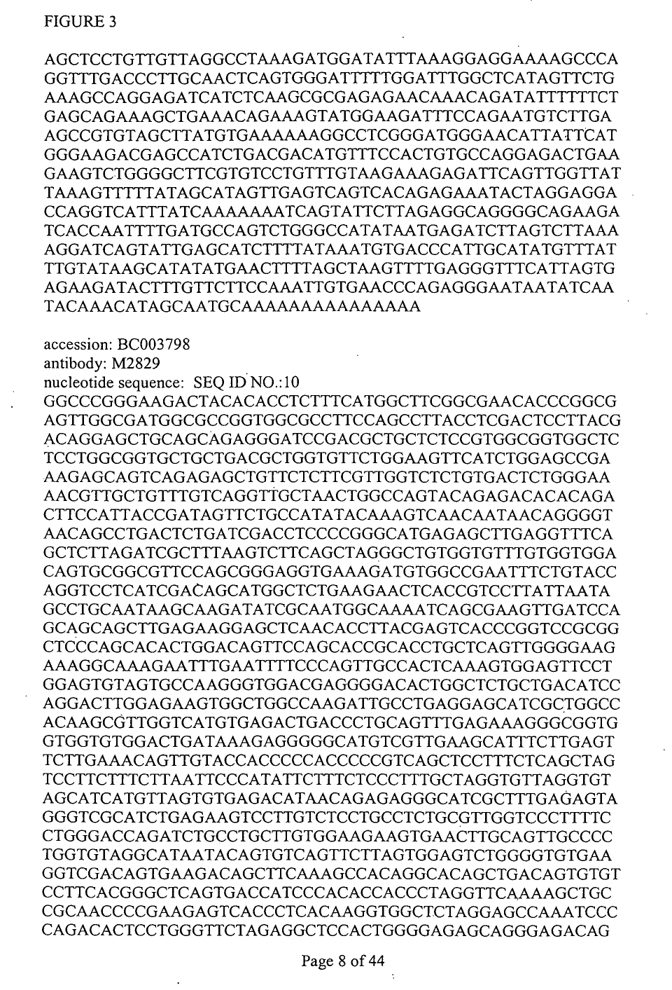

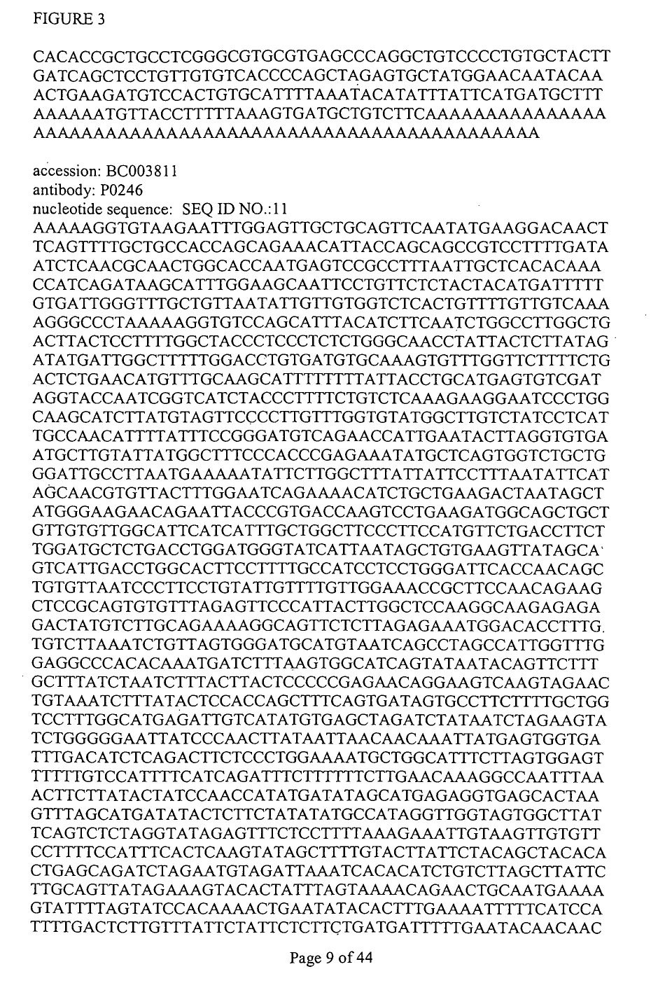

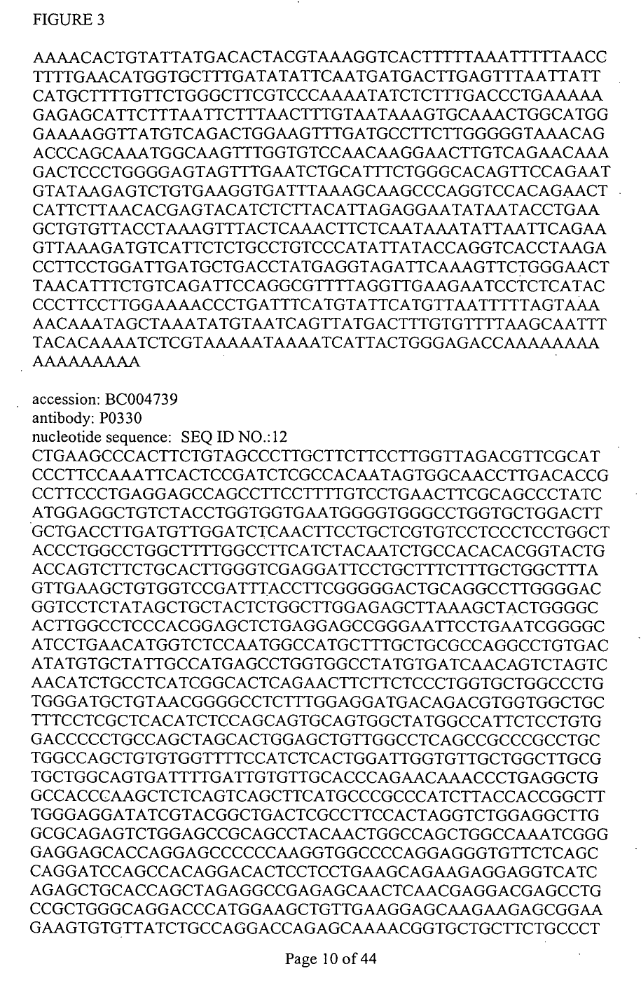

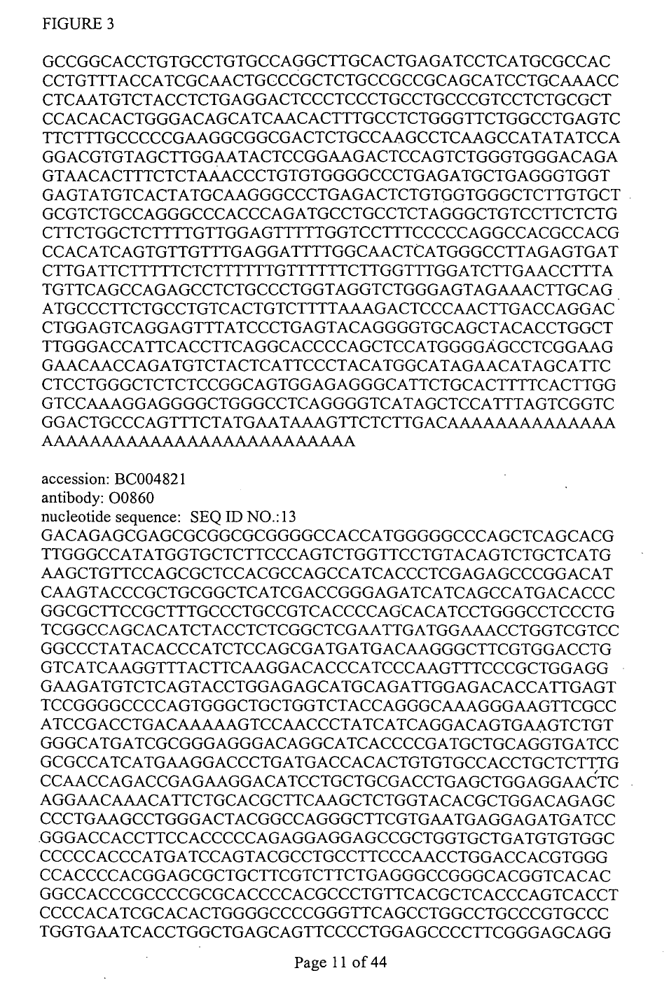

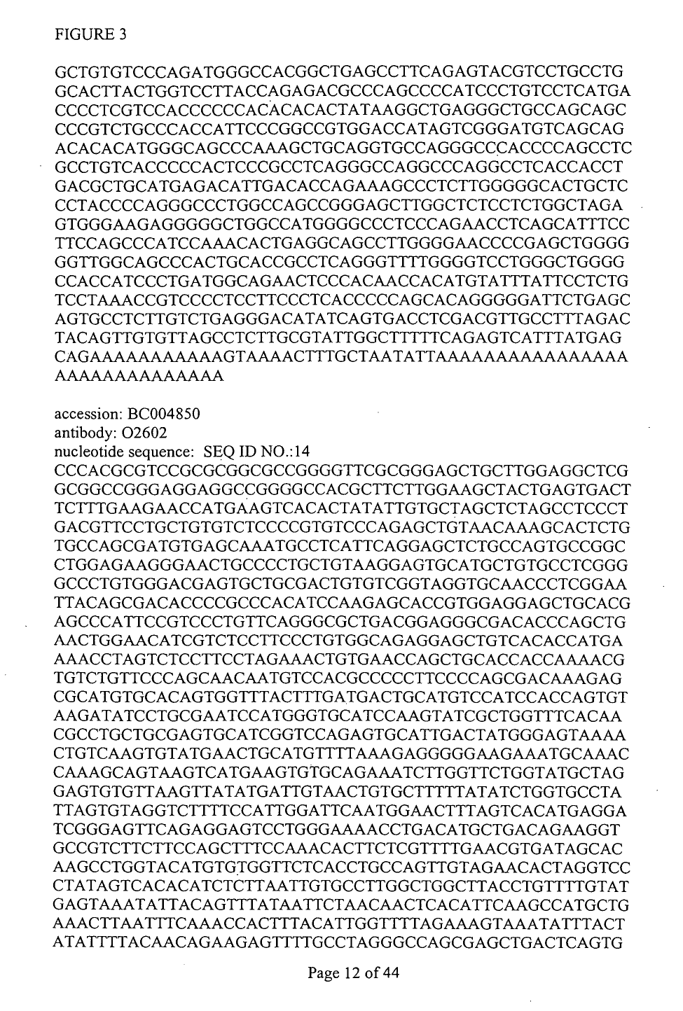

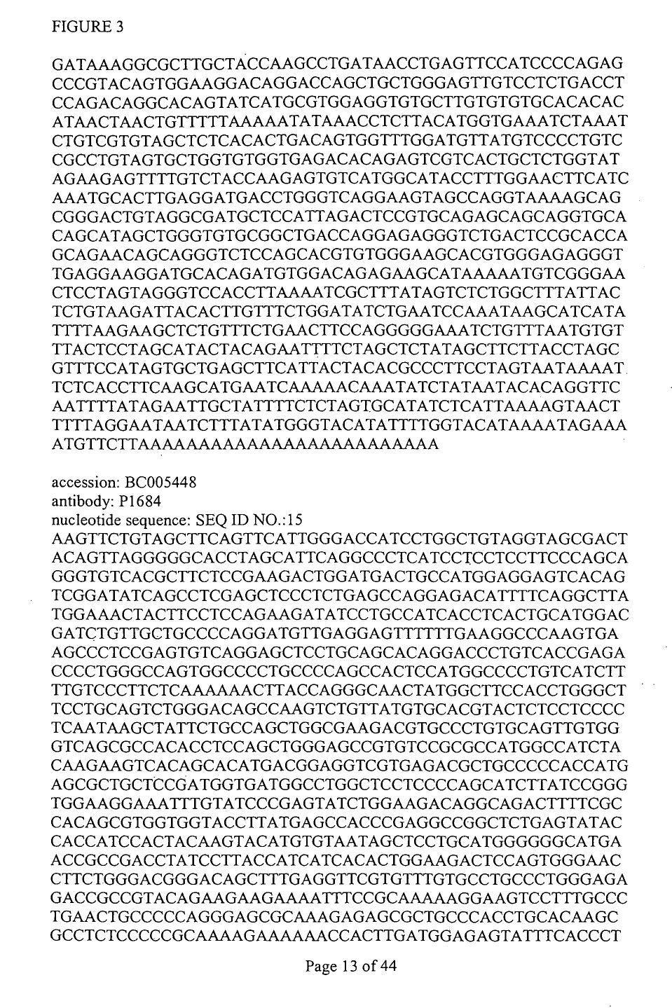

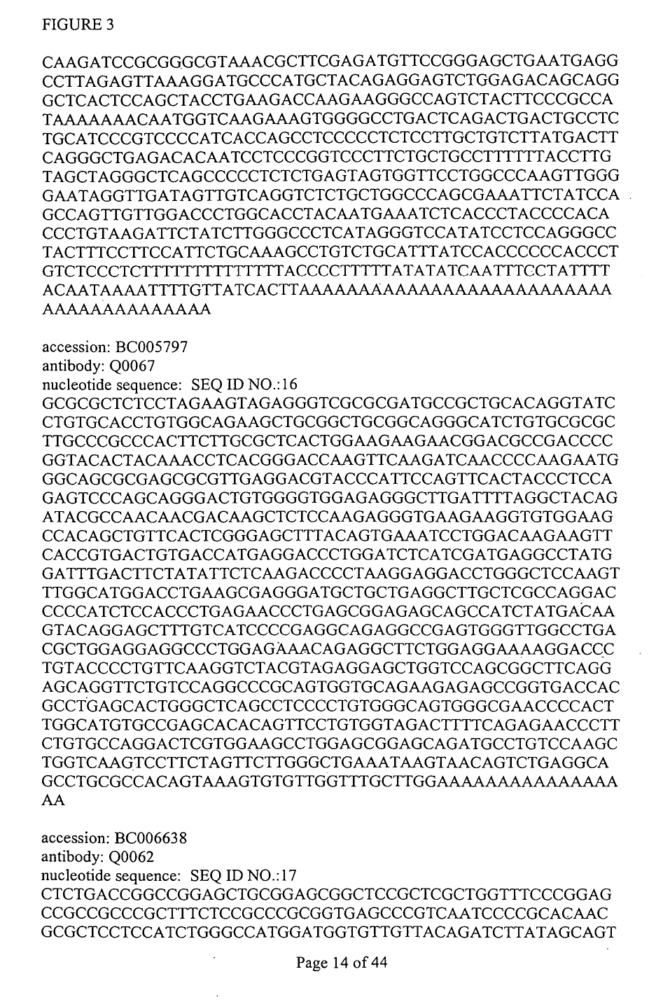

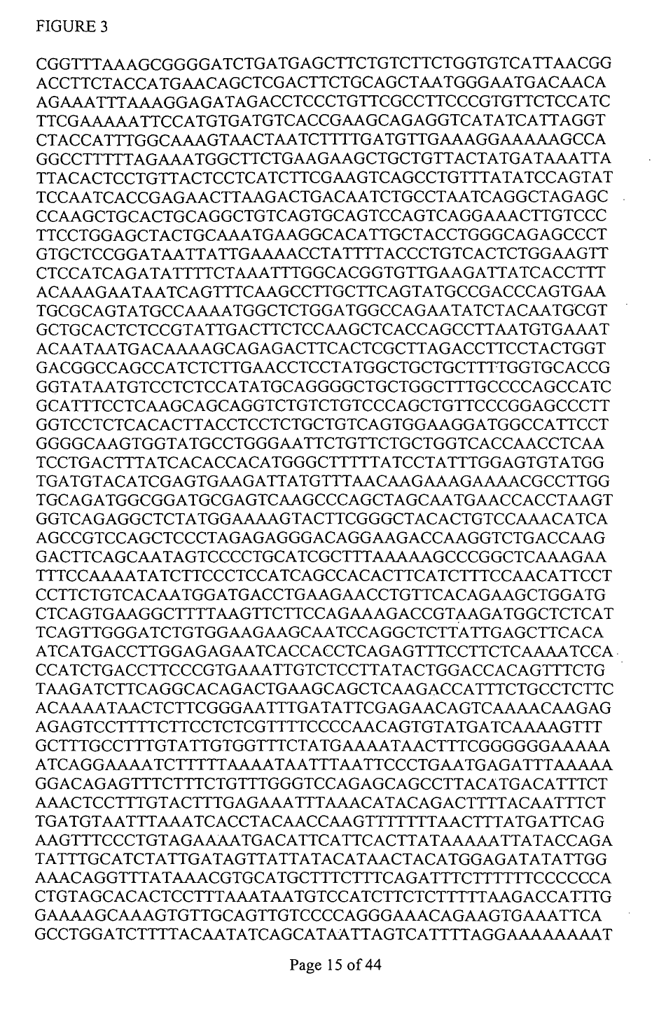

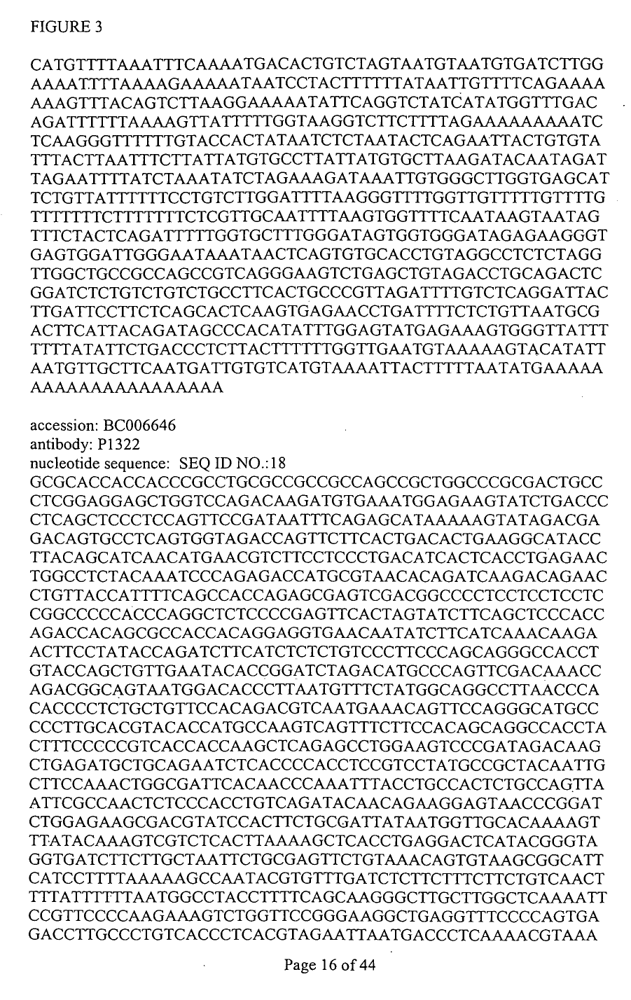

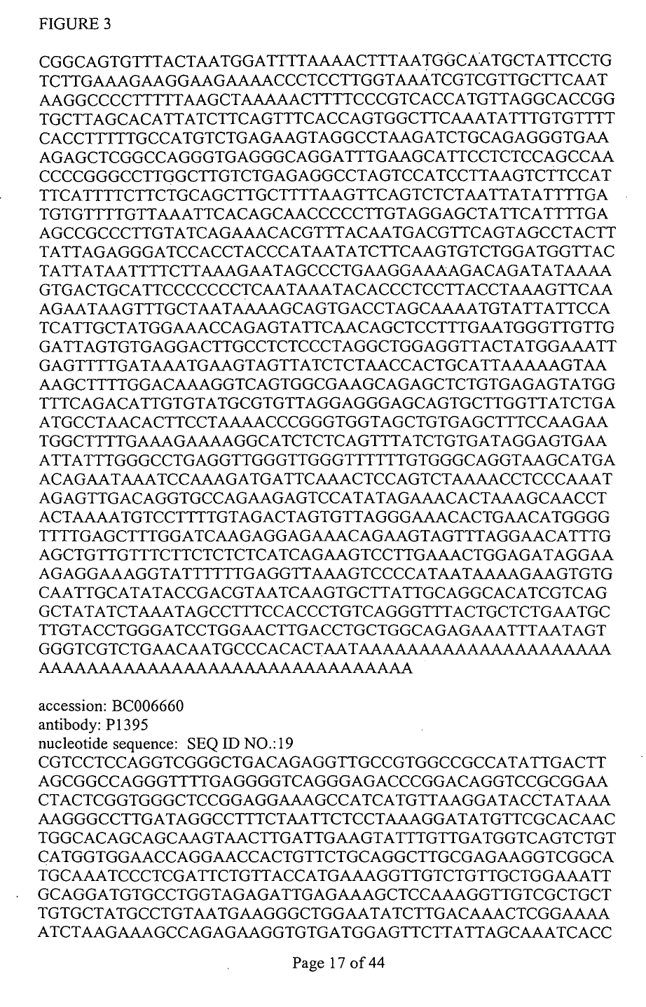

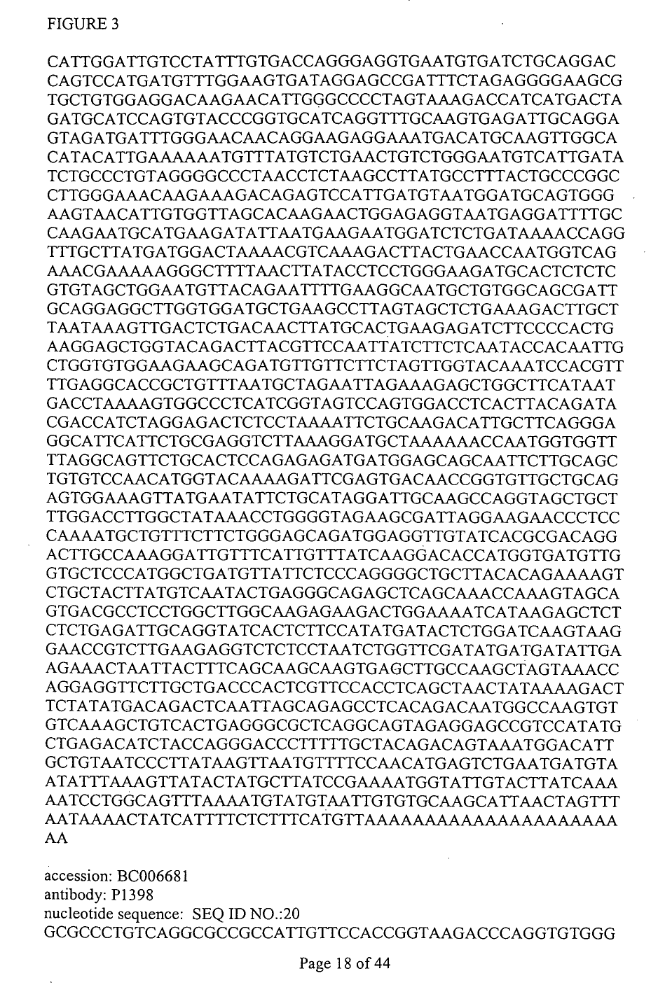

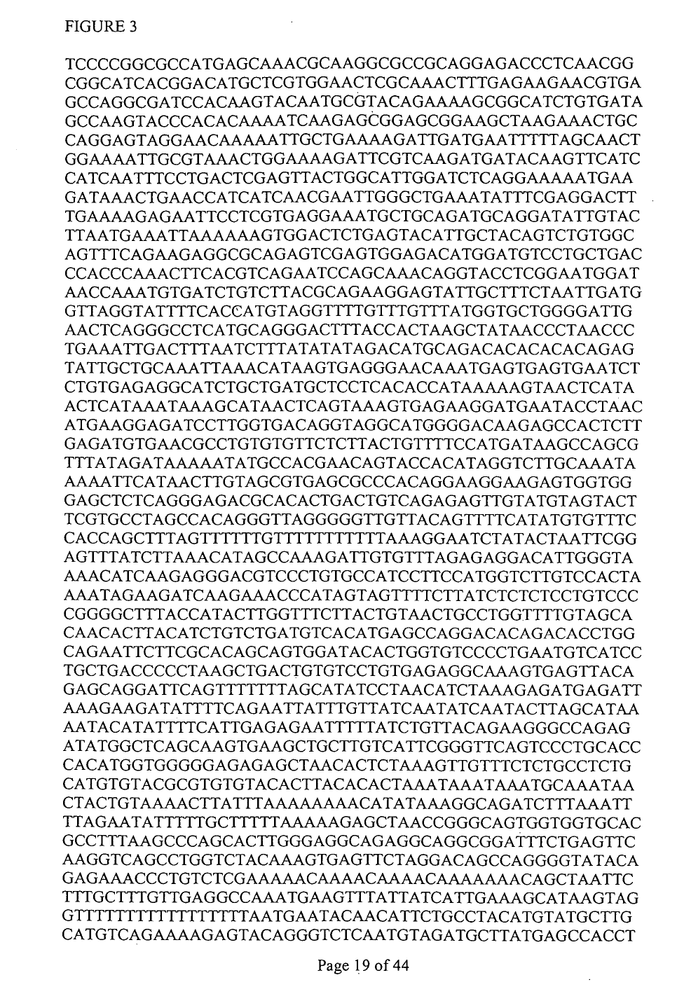

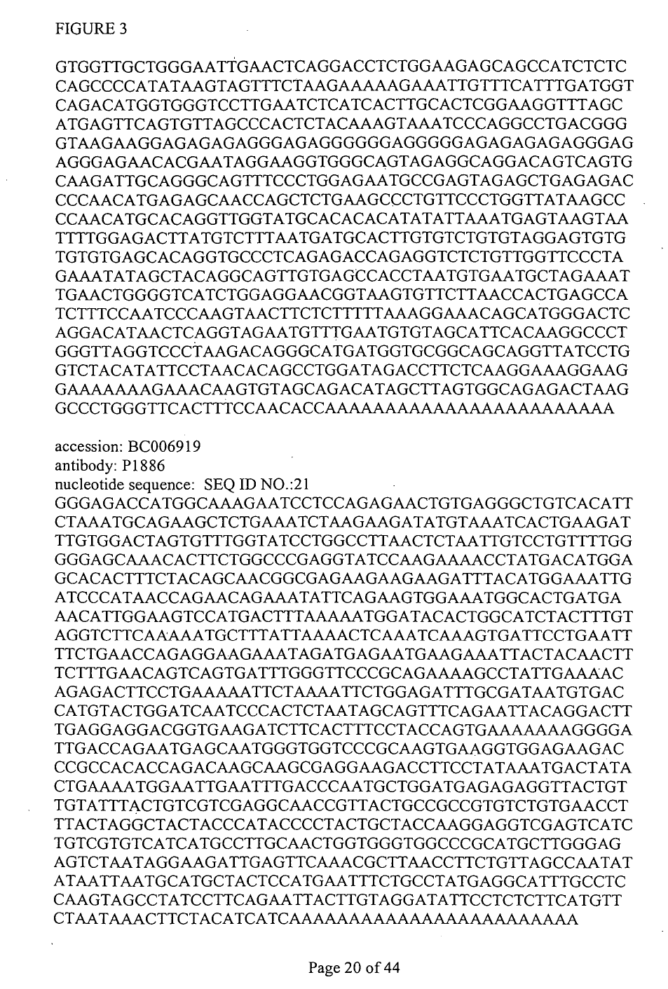

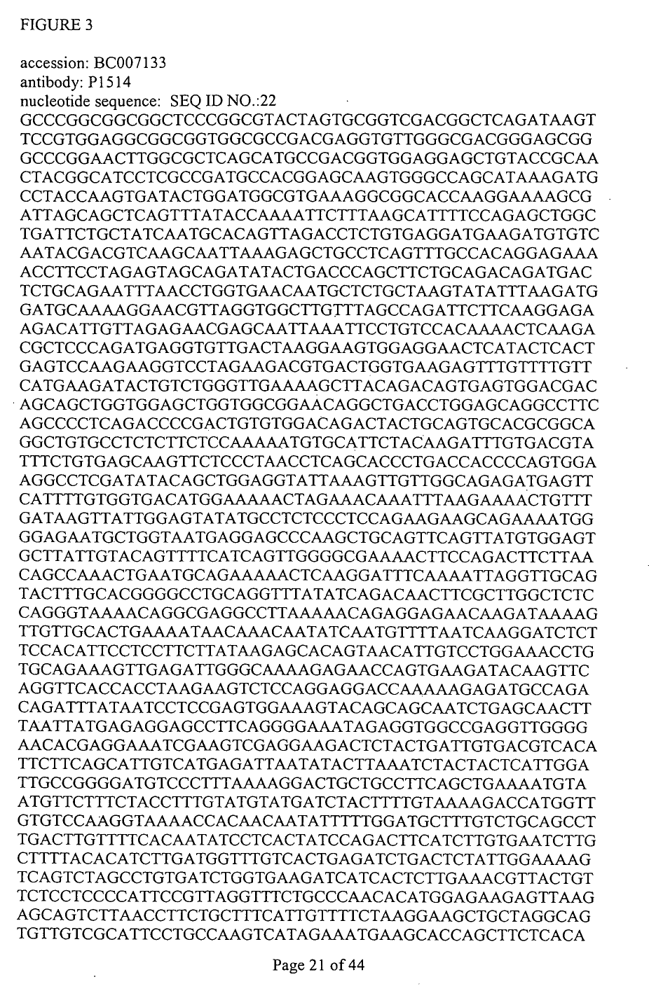

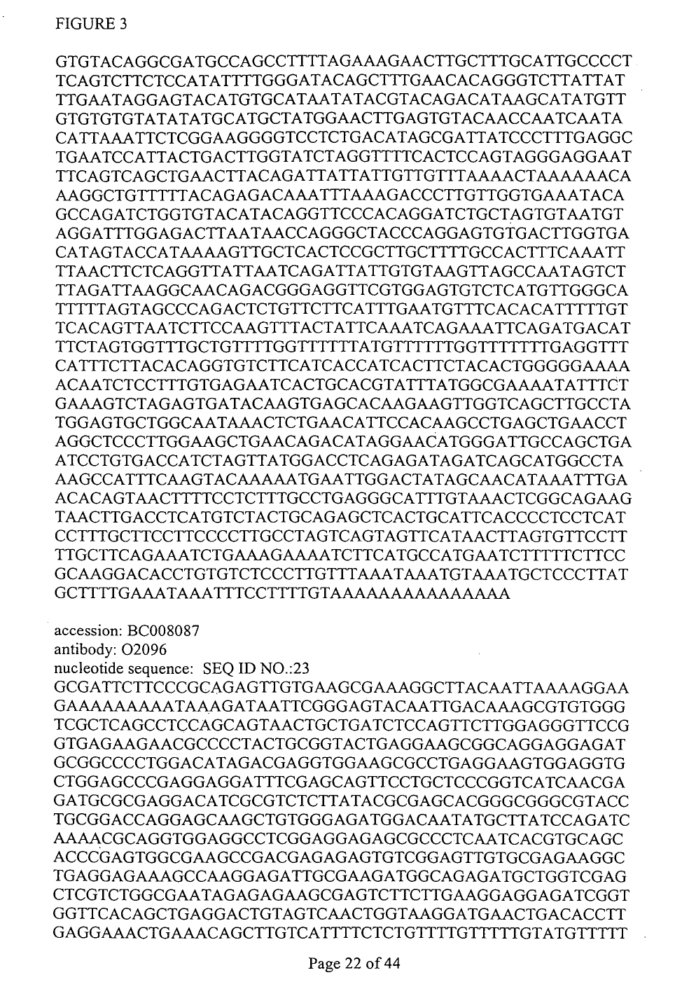

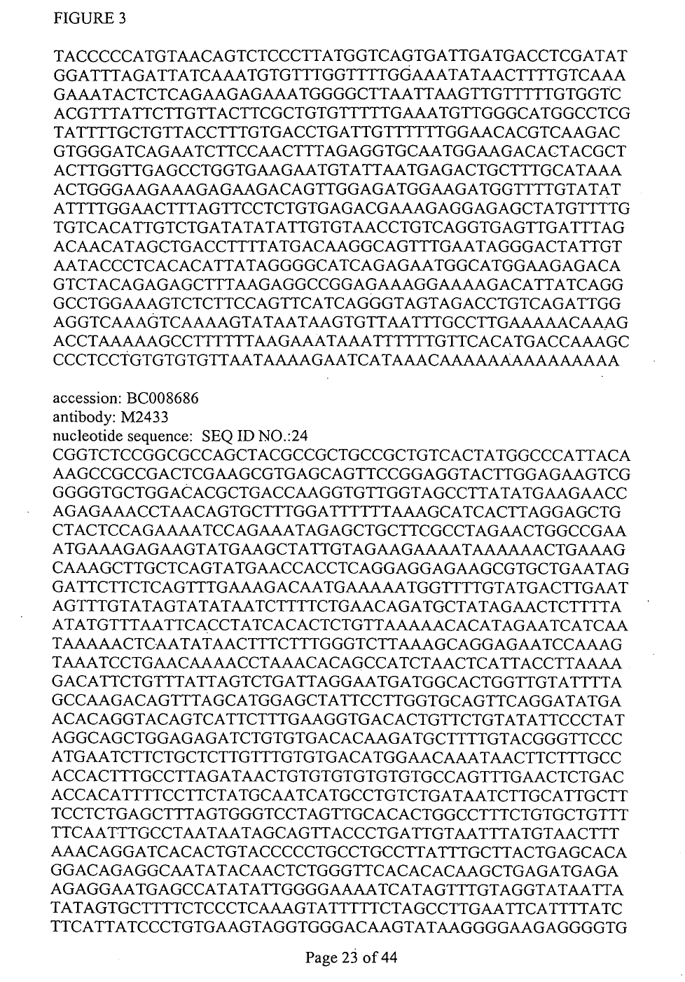

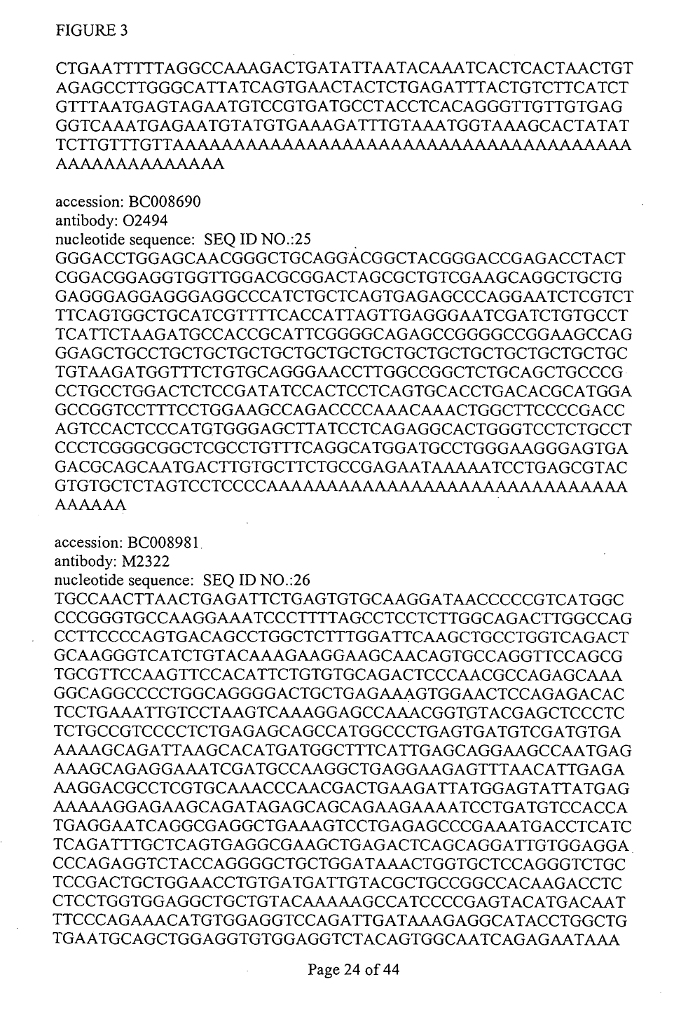

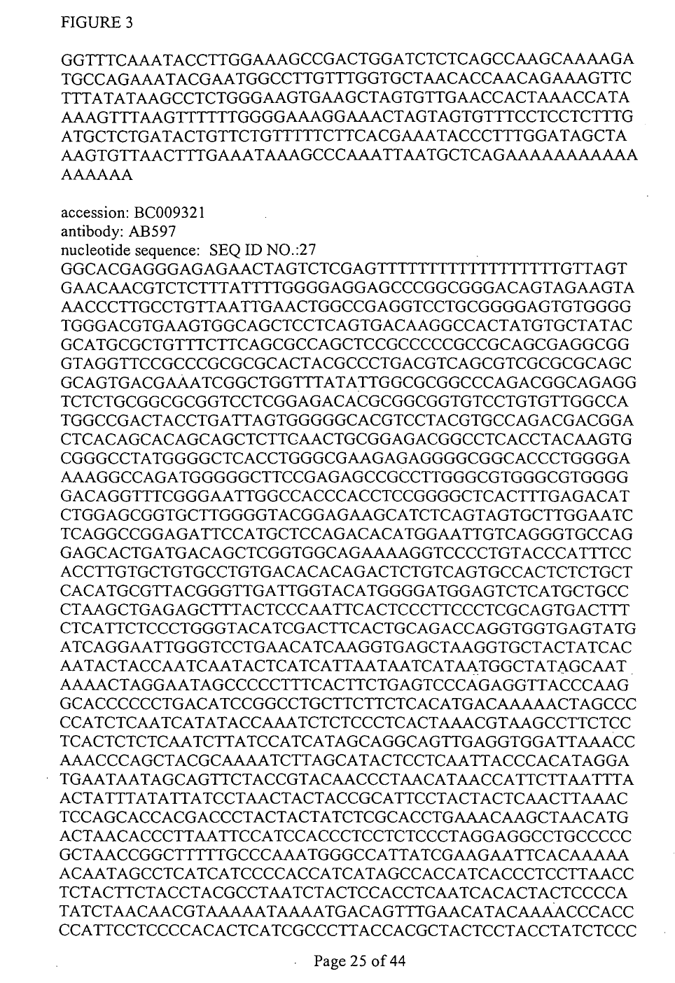

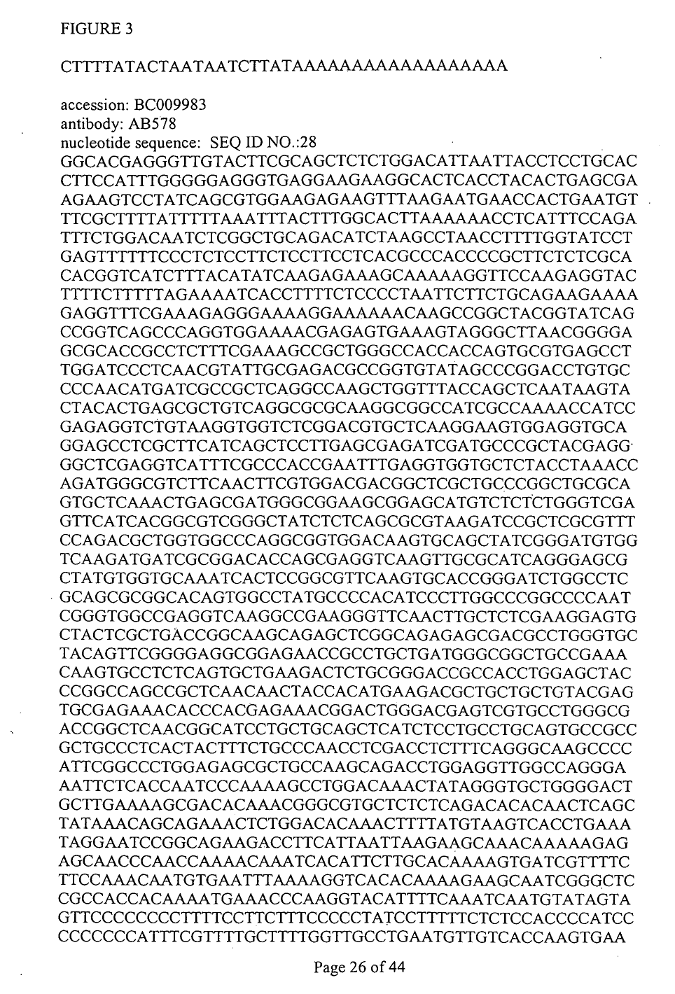

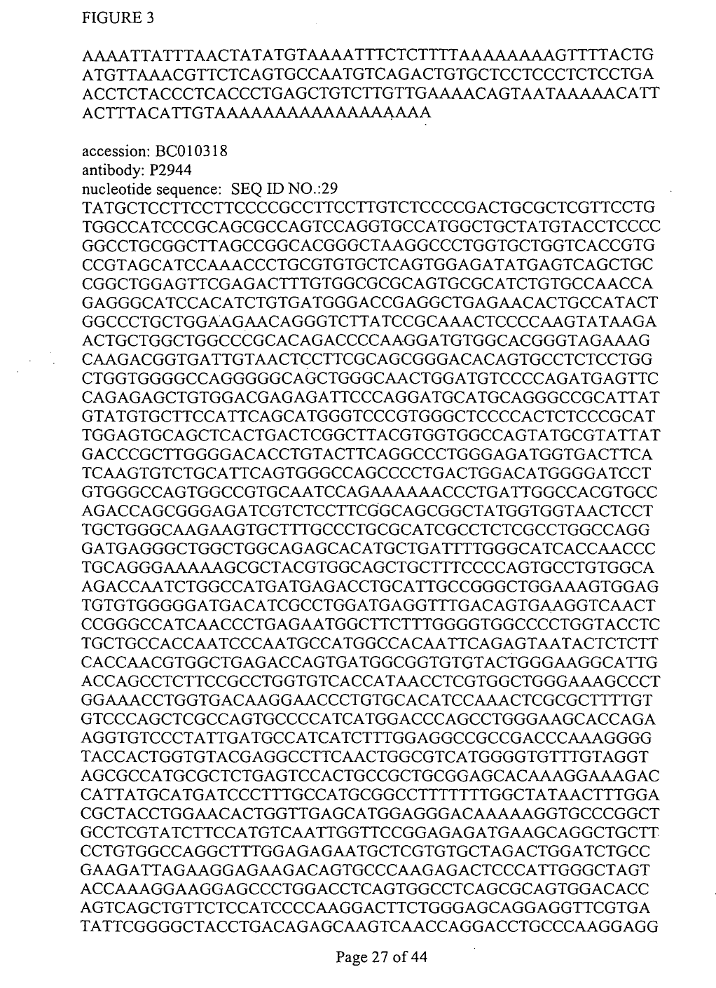

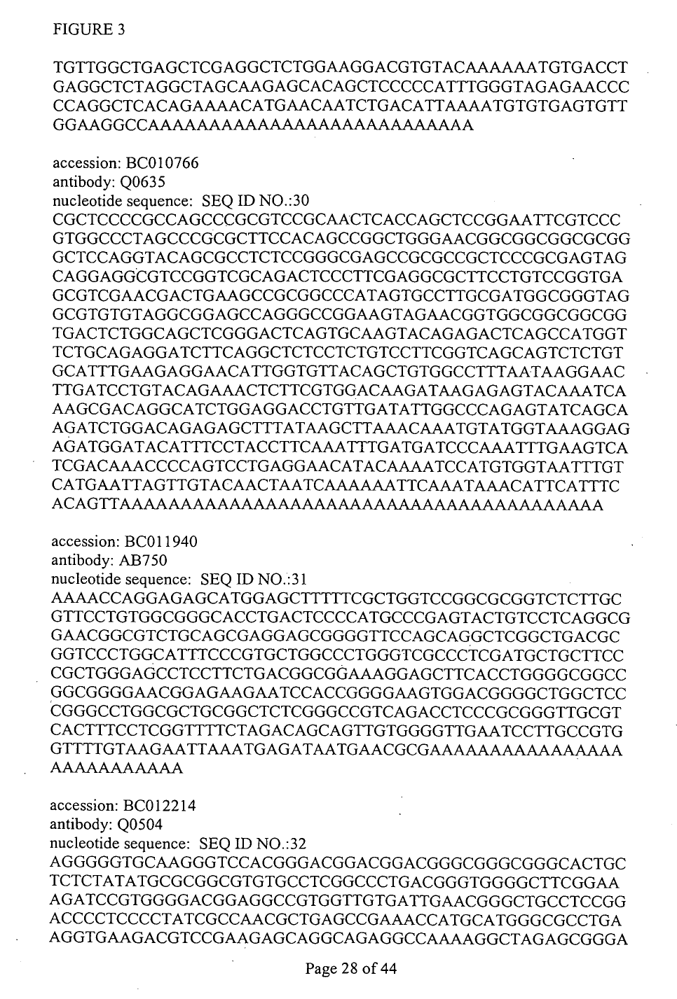

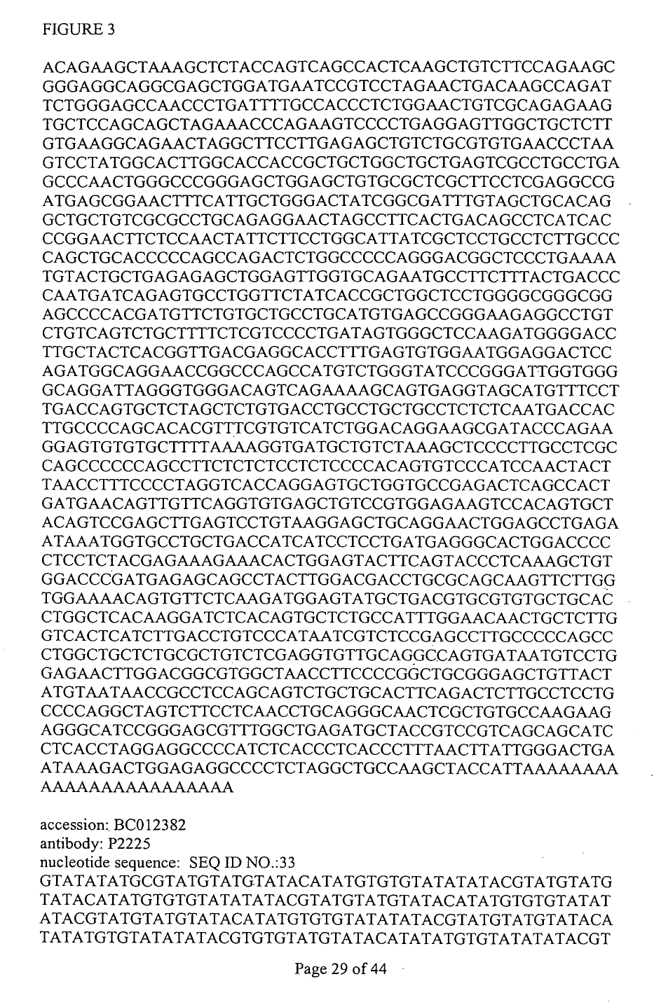

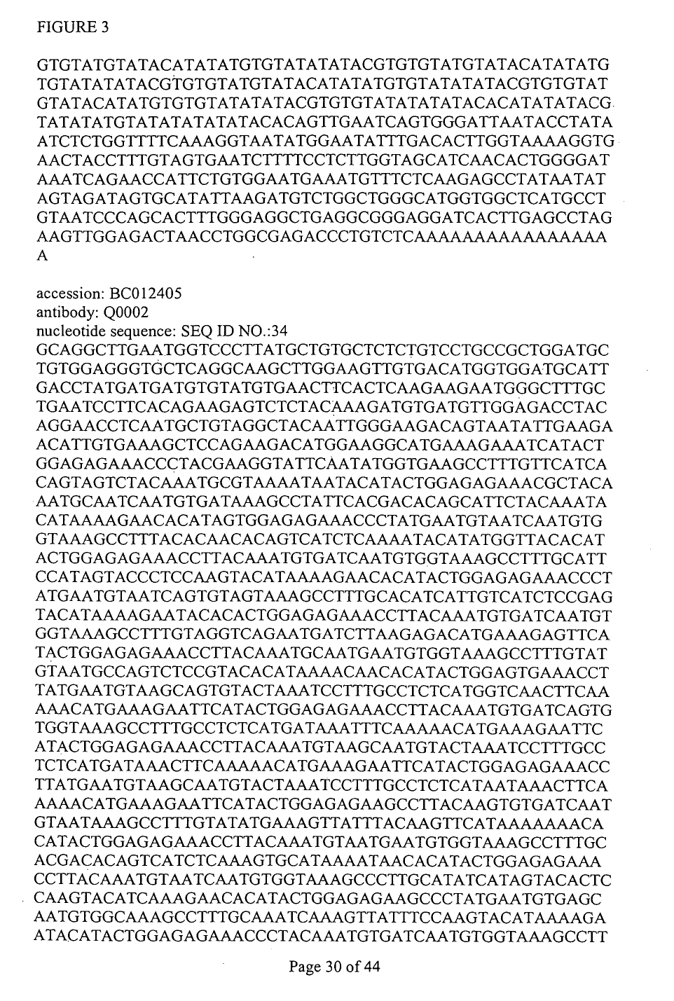

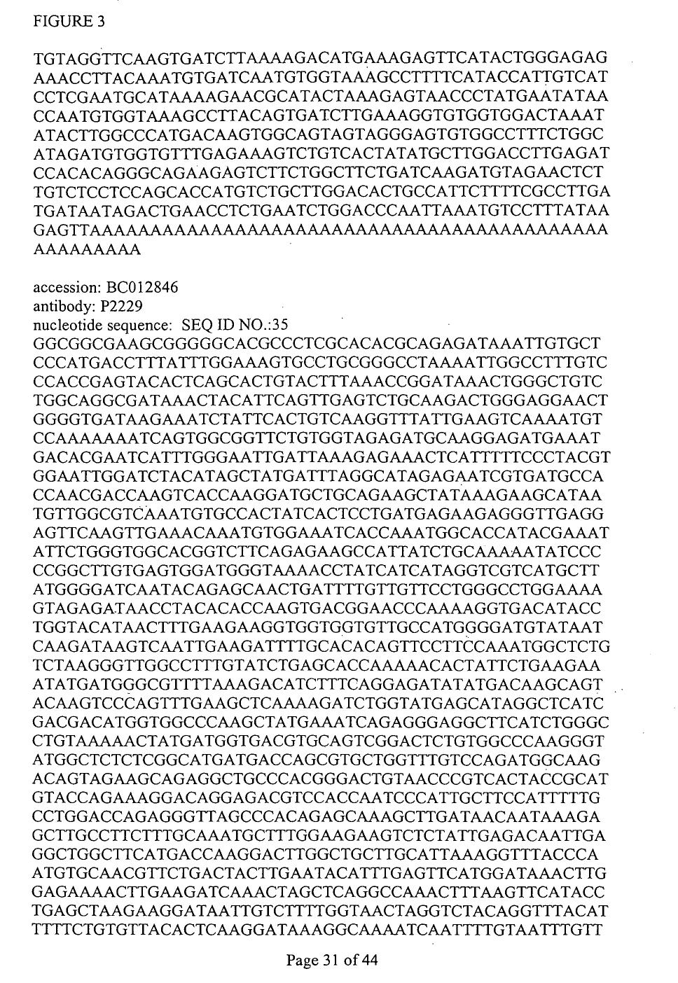

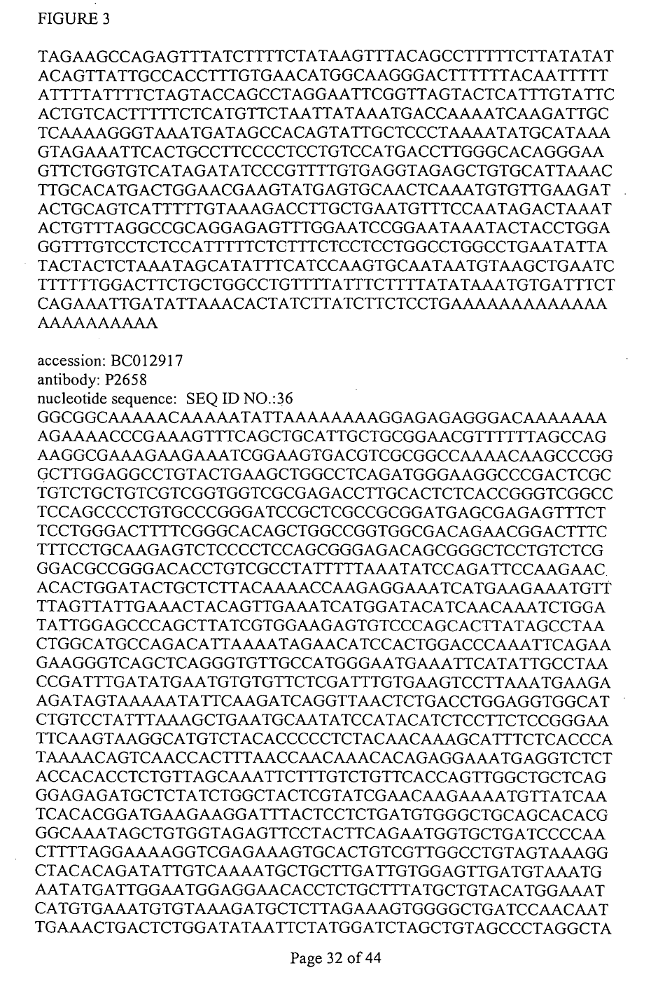

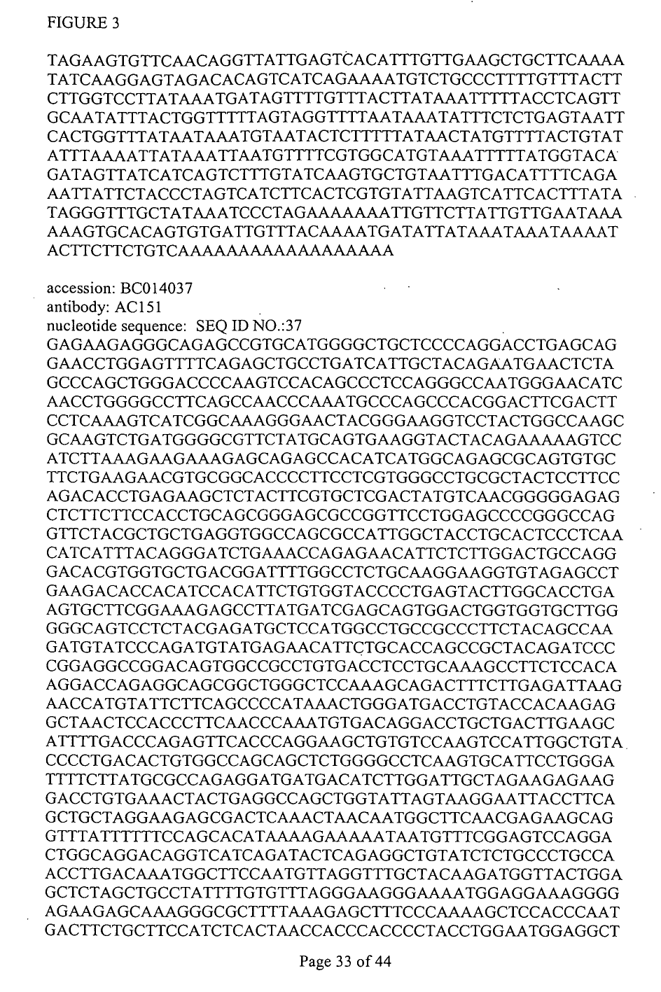

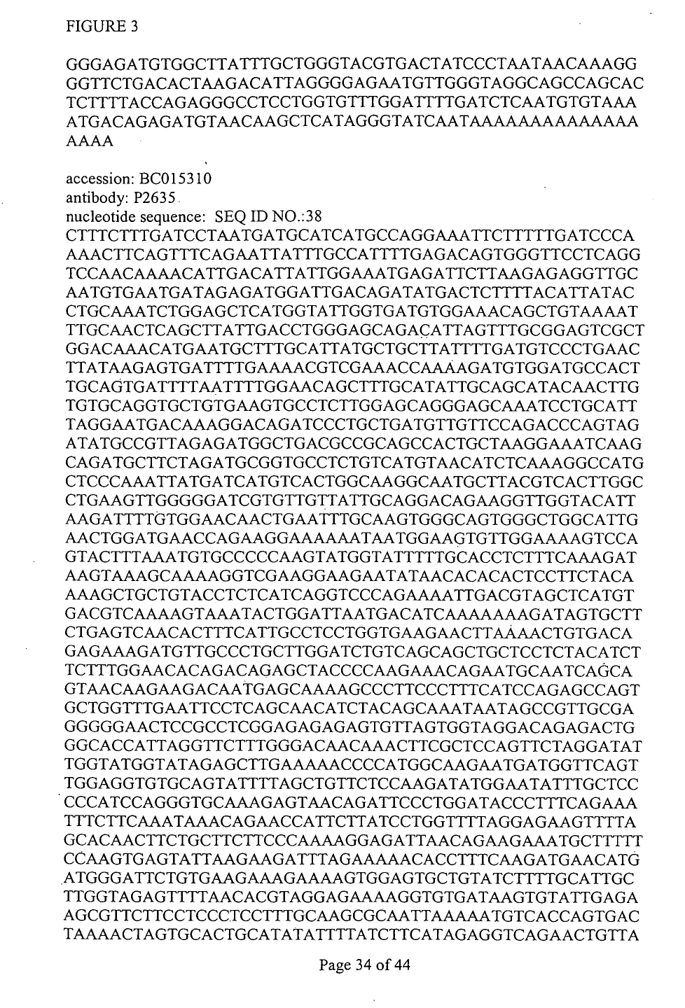

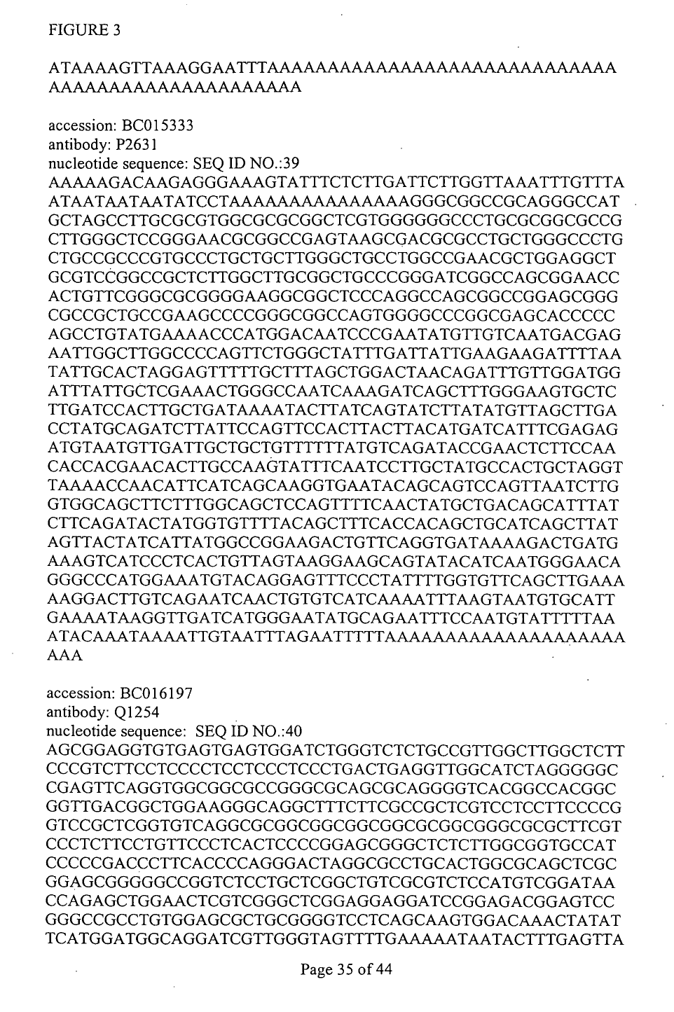

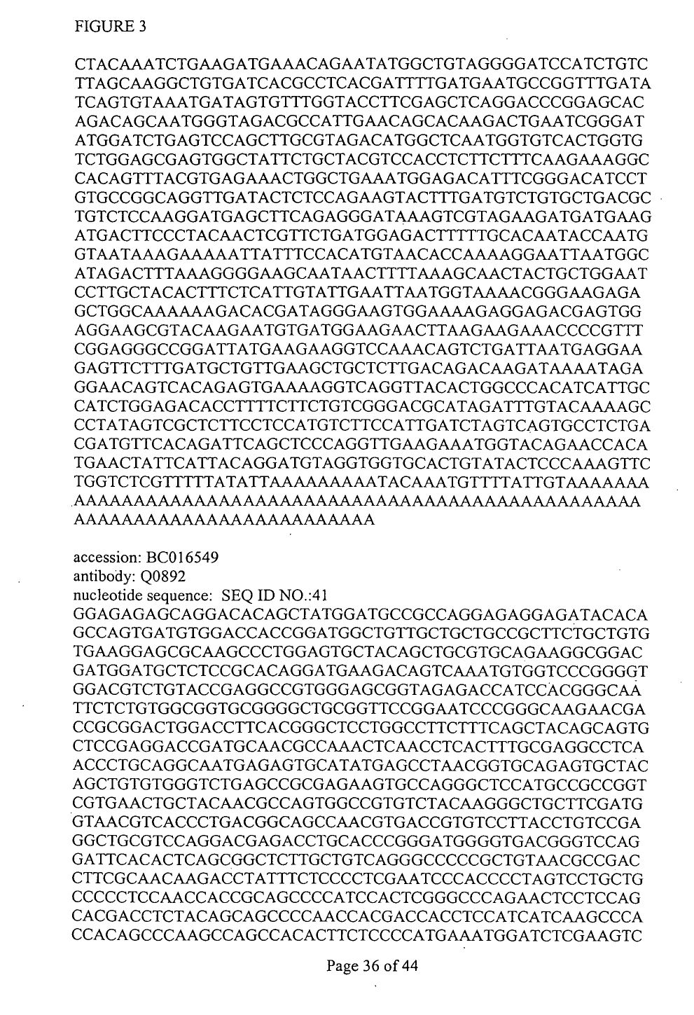

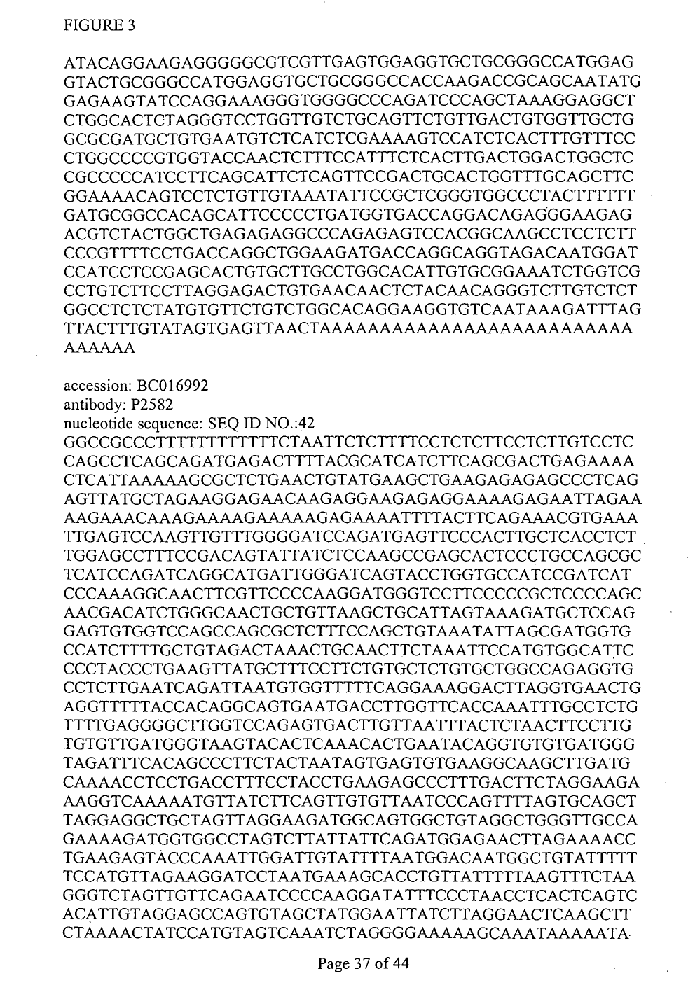

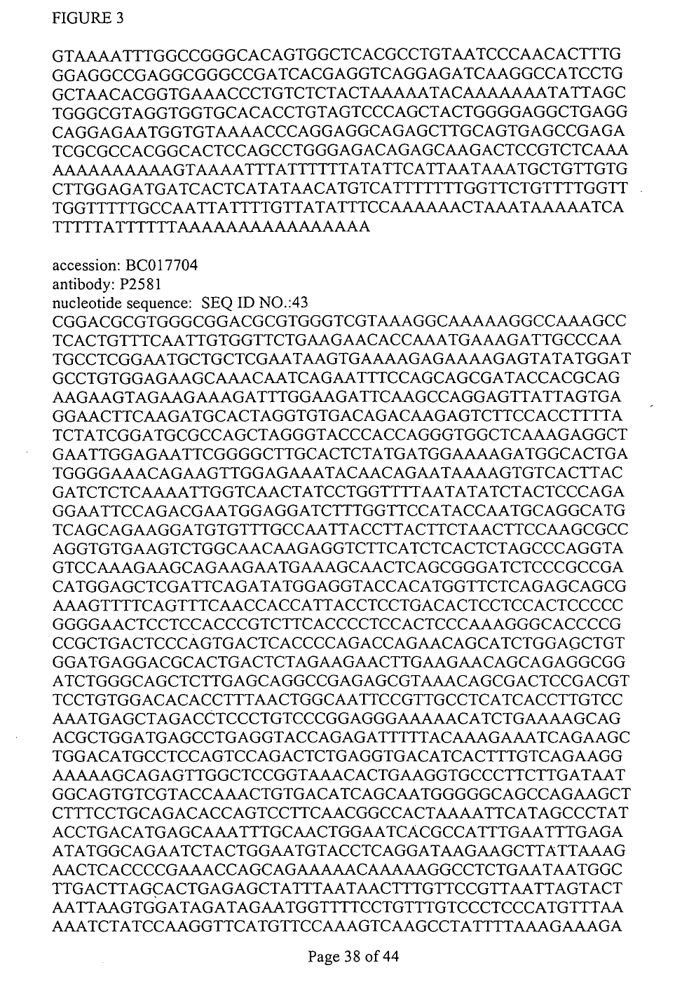

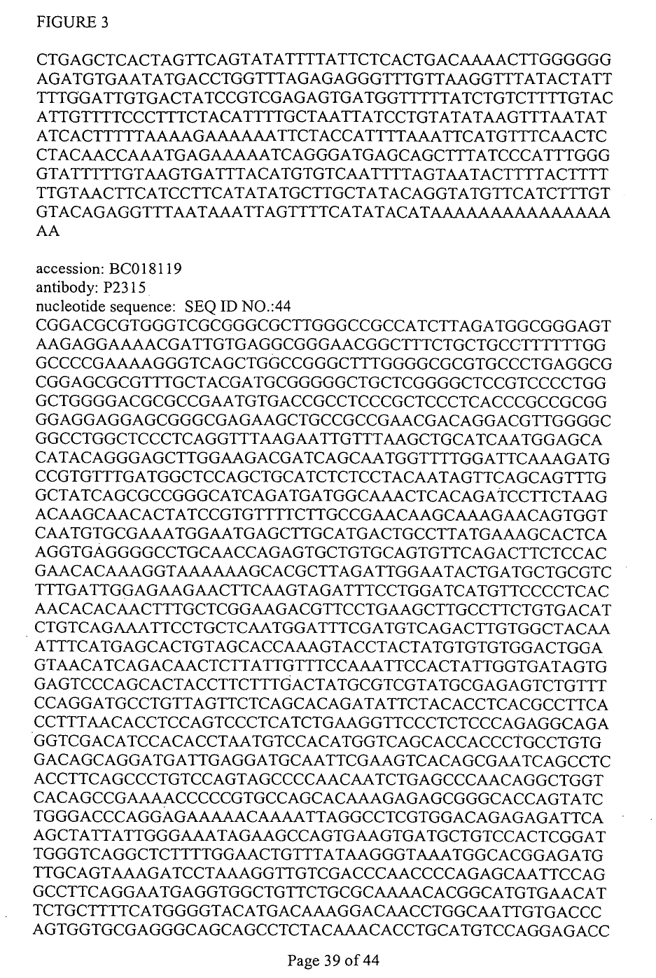

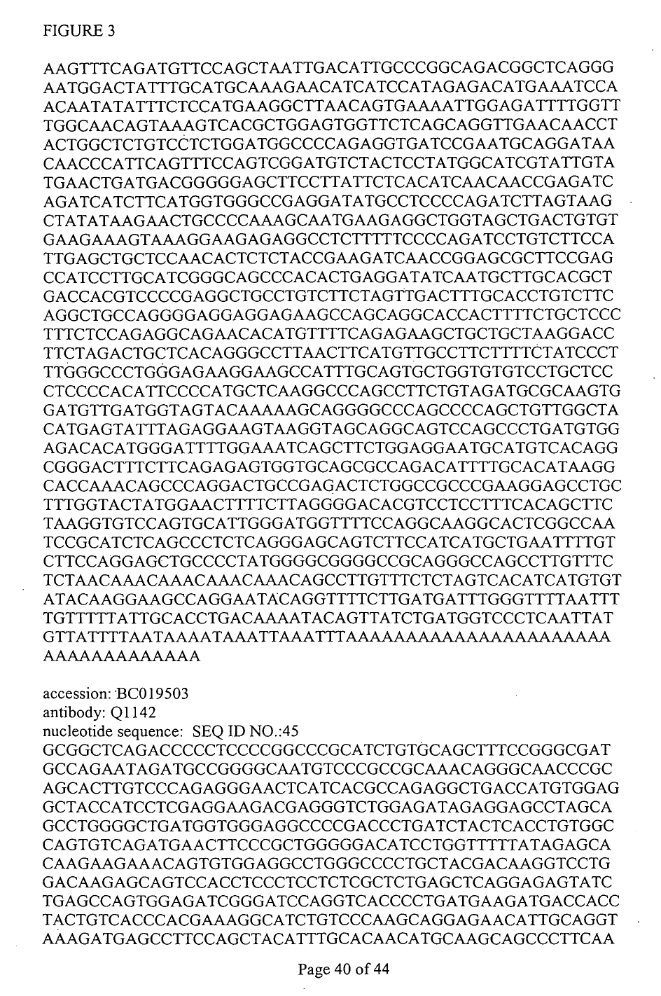

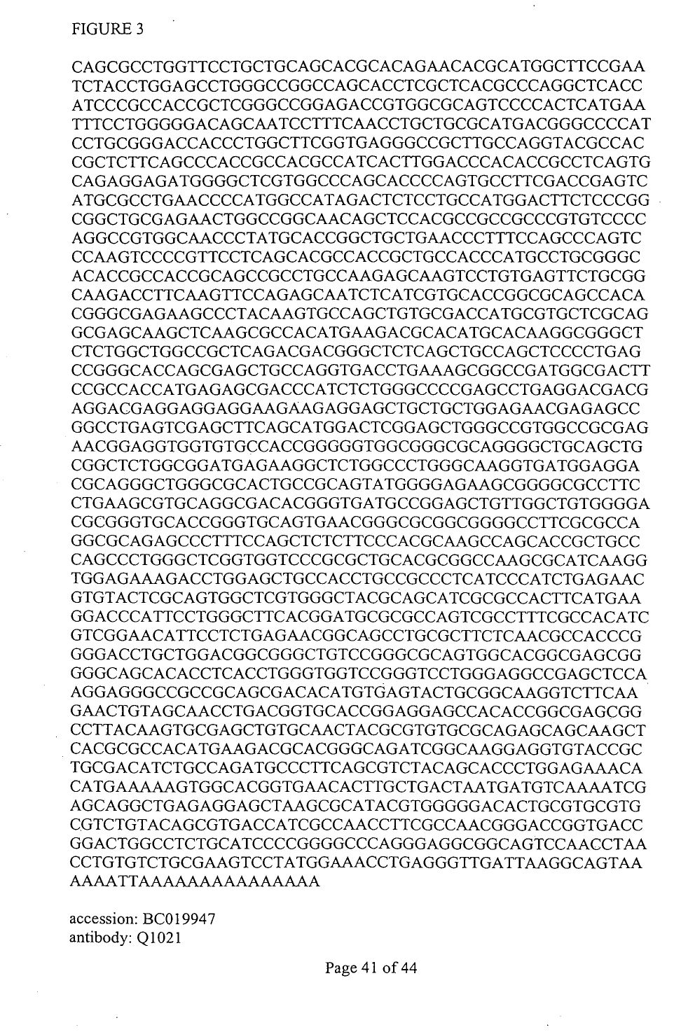

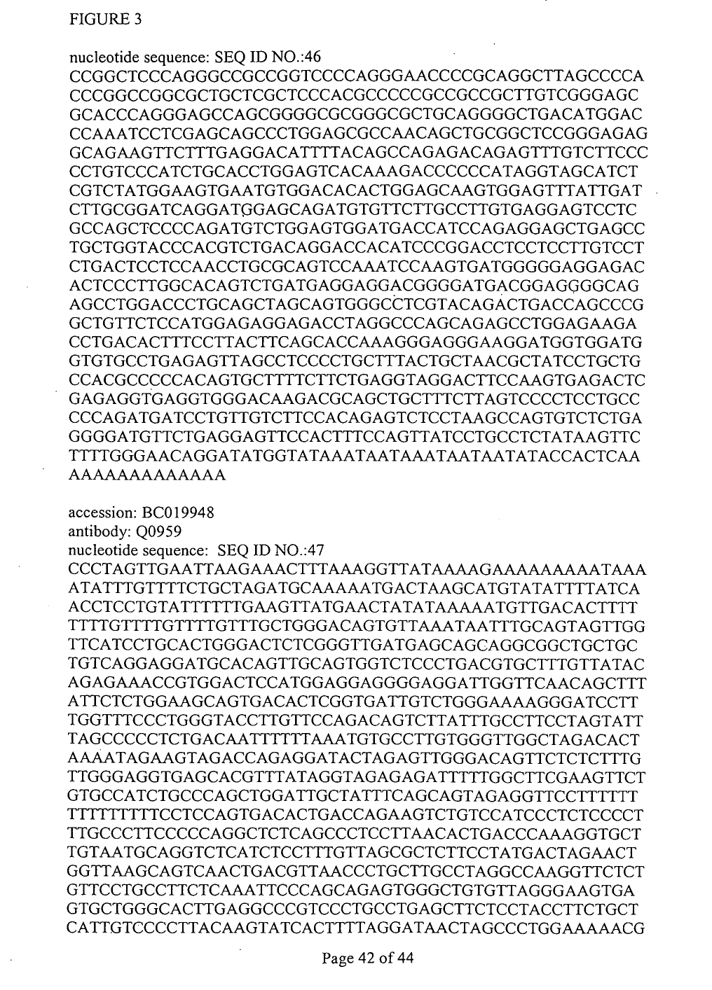

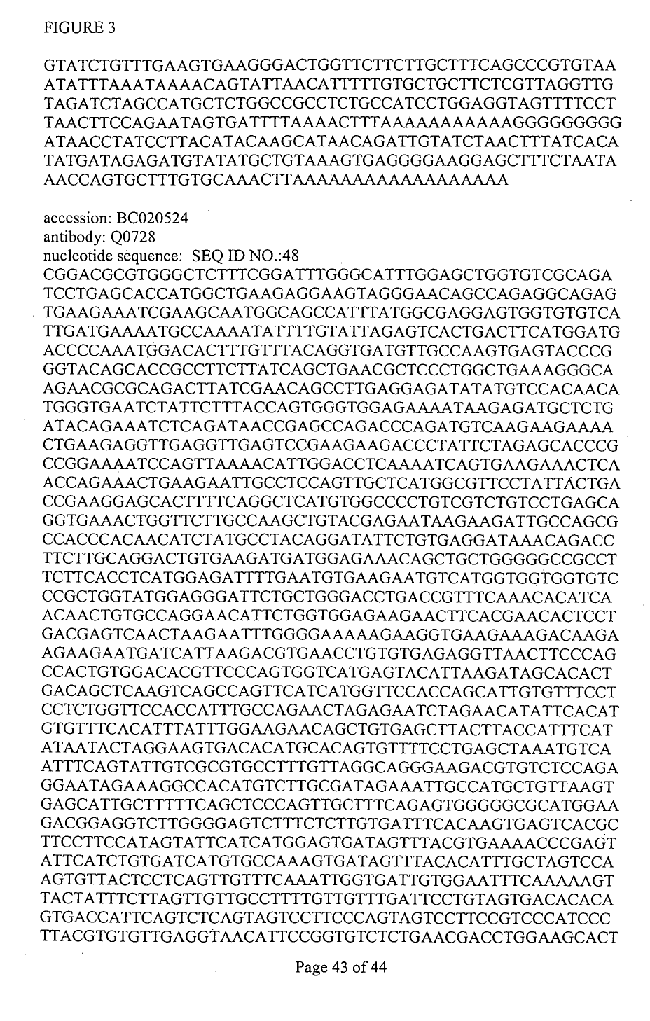

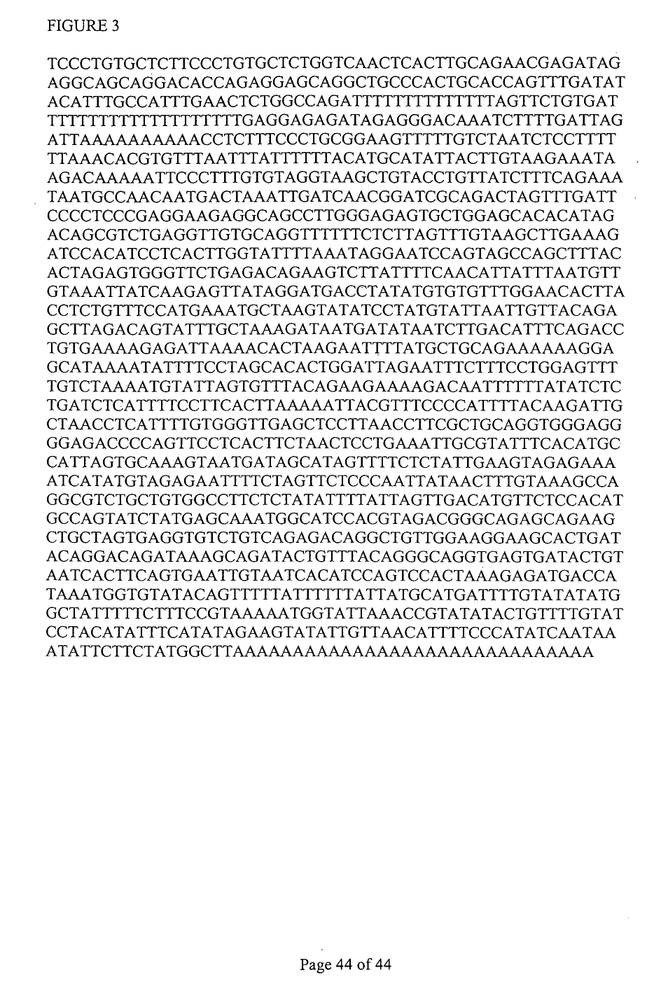

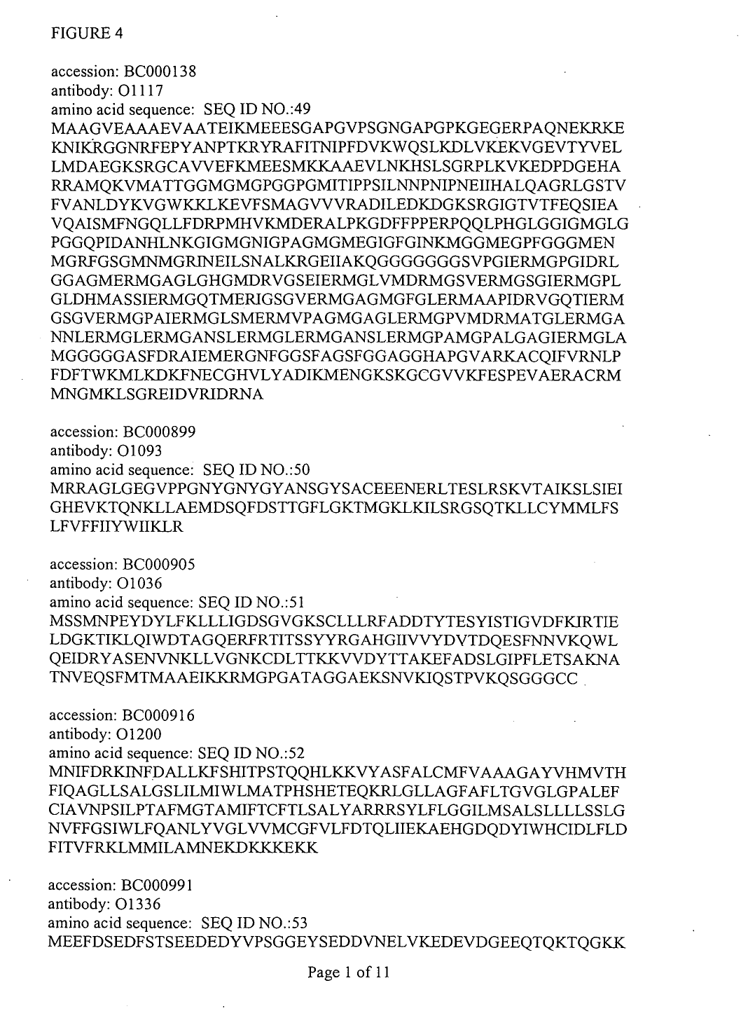

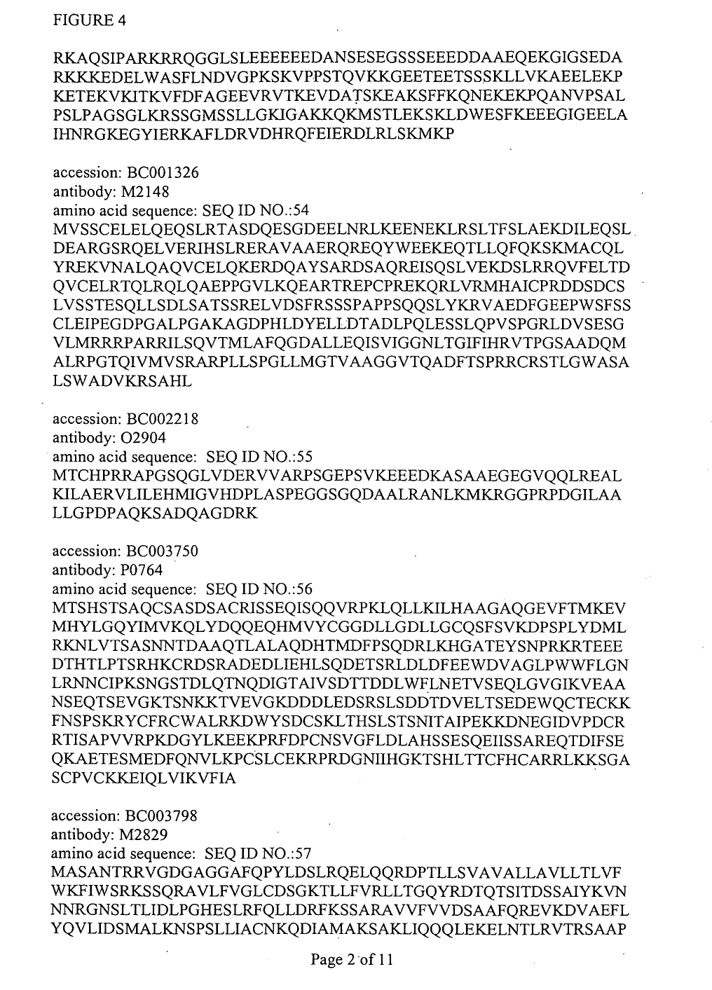

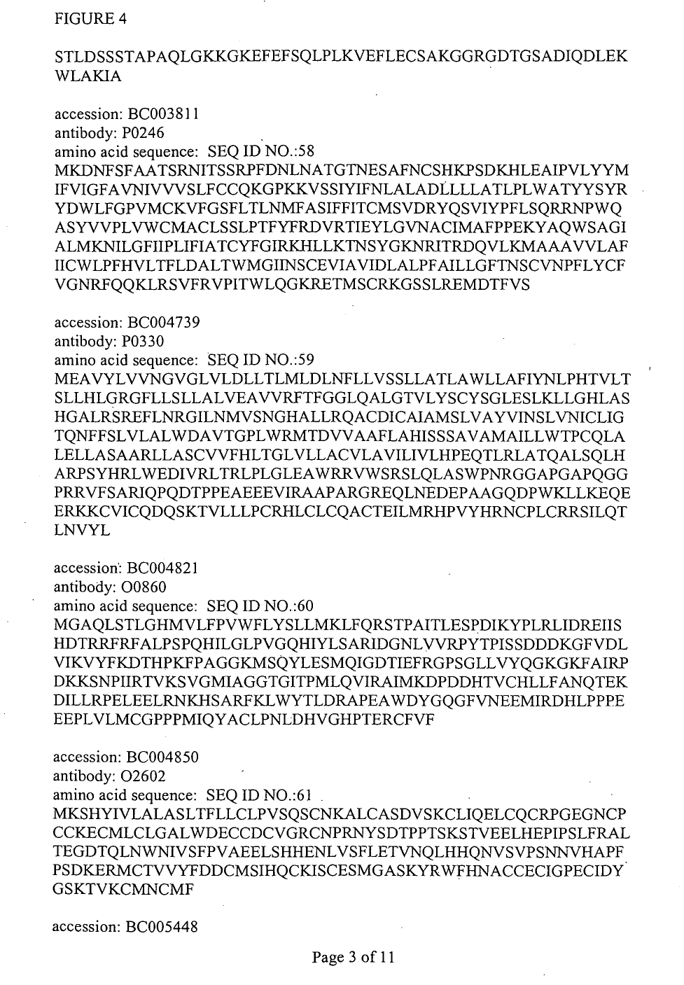

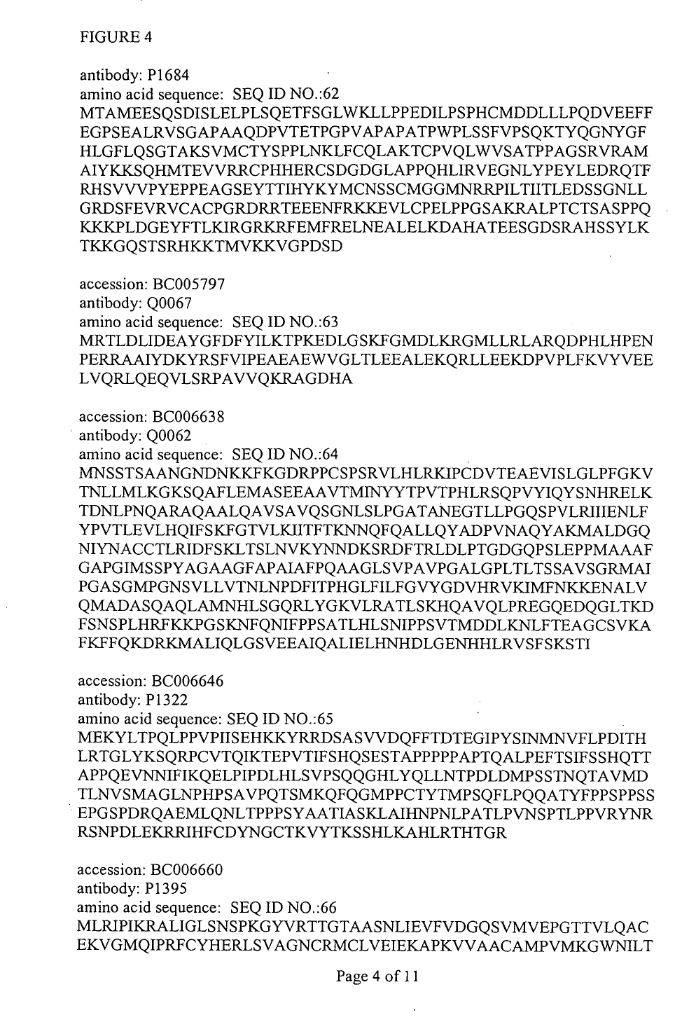

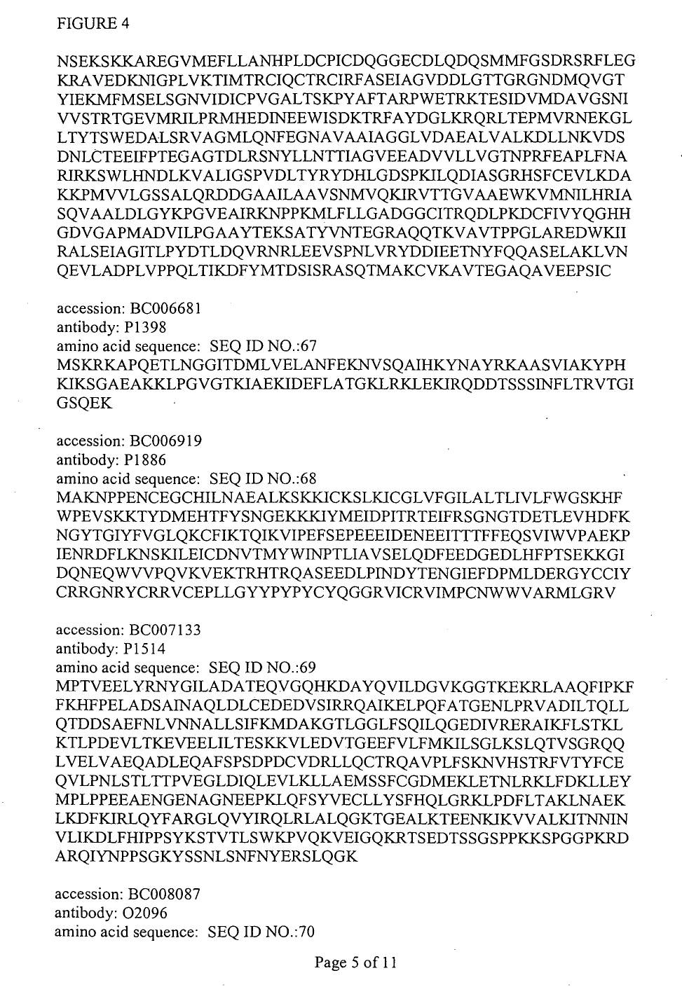

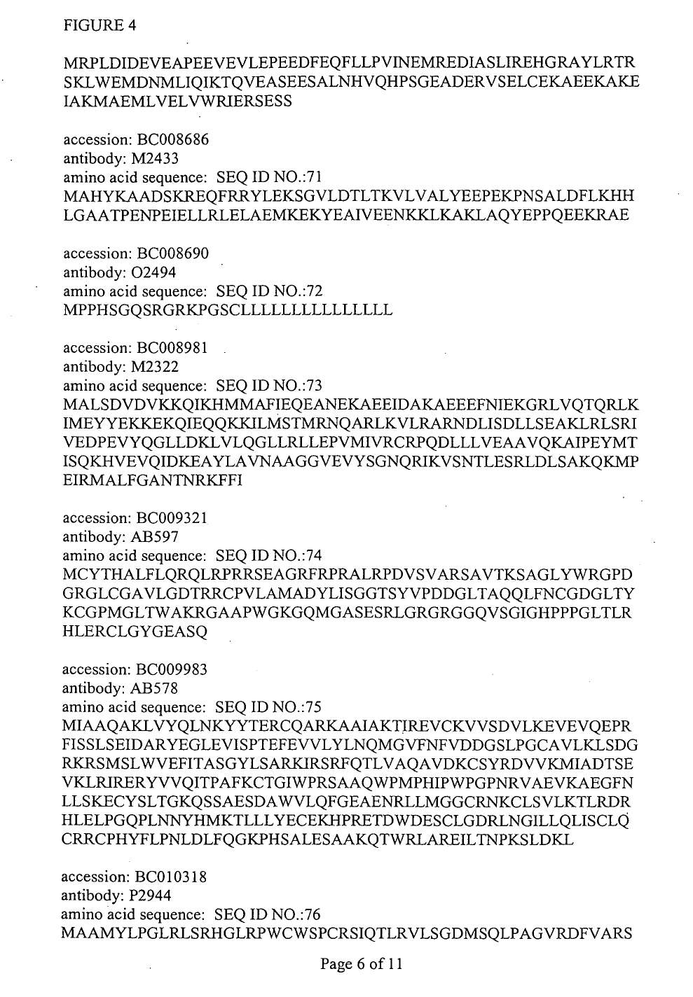

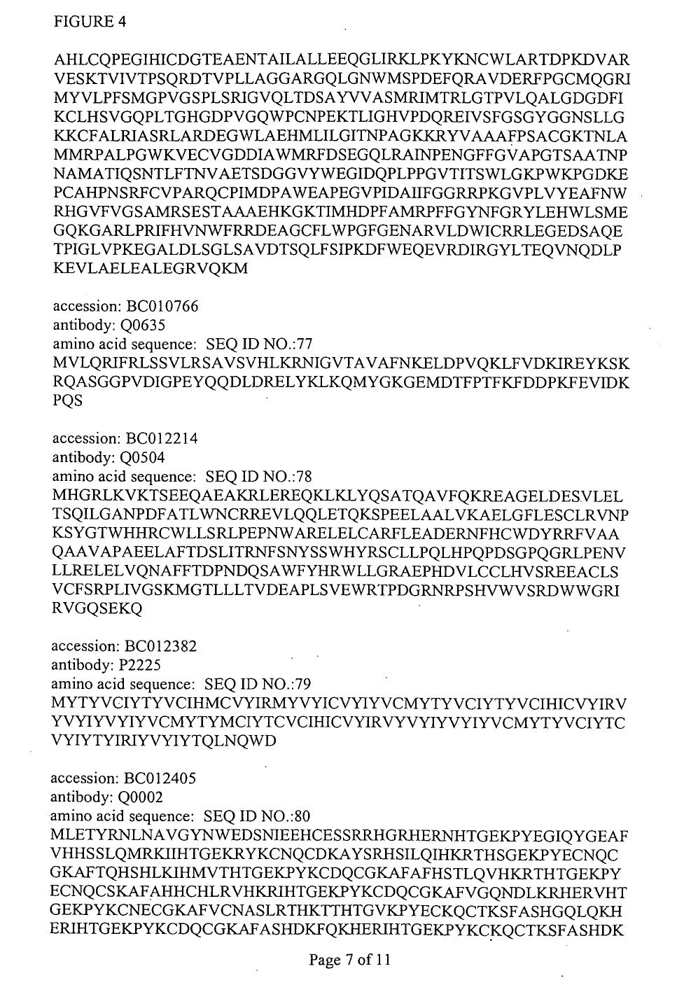

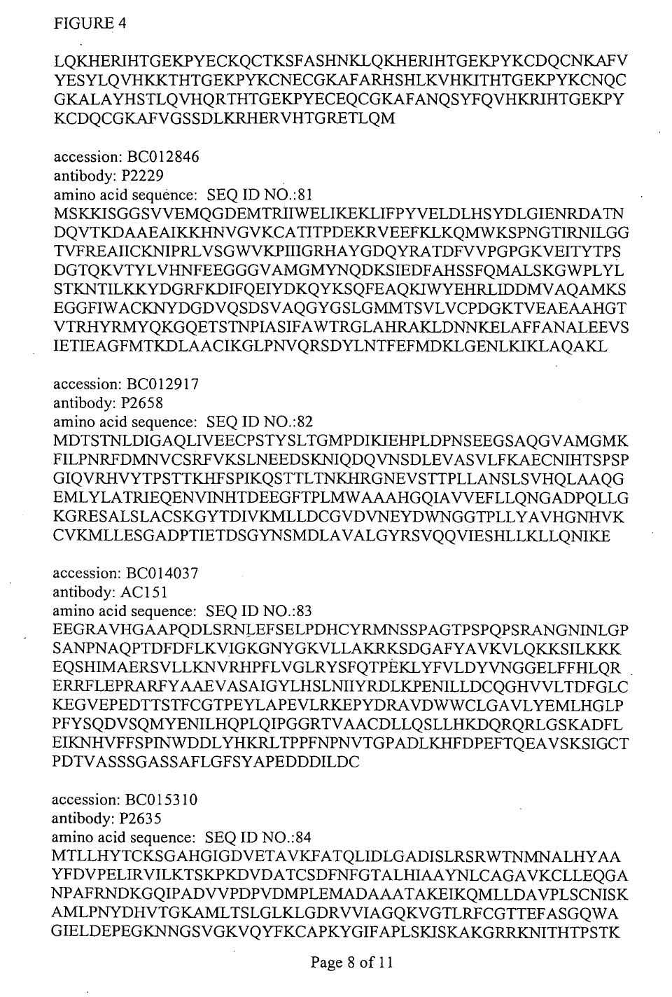

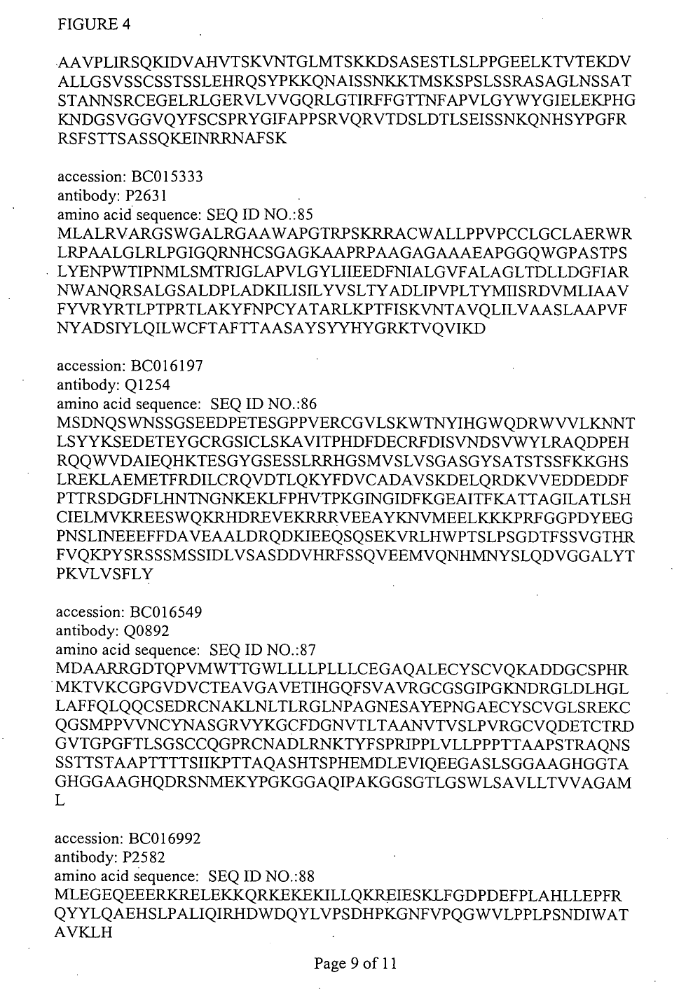

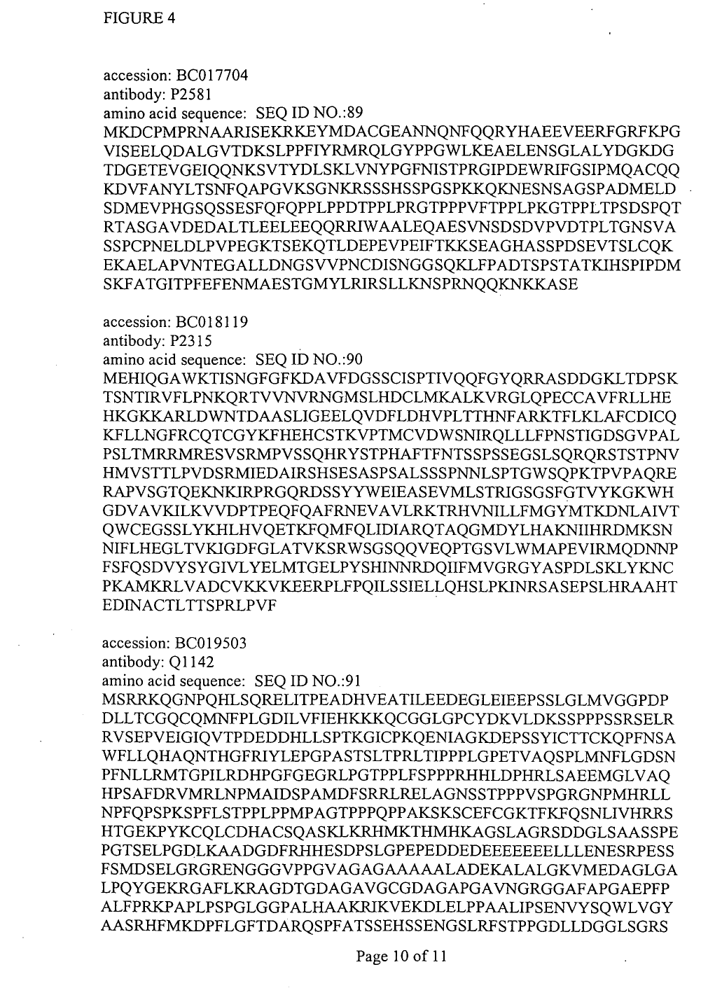

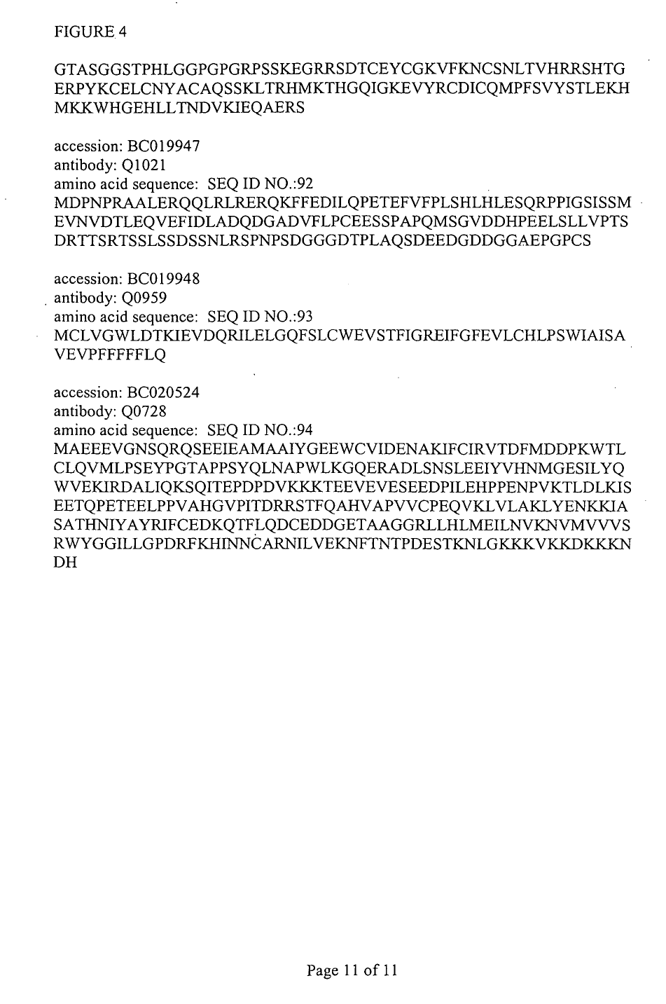

Differential protein expression between normal and diseased samples is shown in a matrix protein array experiment using selected antibodies of the present invention. Plasma samples from lung cancer patients and normal counterparts are printed, in the same protein amount, in each chamber of the matrix protein array. Each chamber displays the same matrix of samples. Protein samples used in this experiment are referred to as “protein set 6”, where plasma samples are from normal controls and lung adenocarcinoma patients. Table 1A provides detailed clinical information on the protein set used, such as the lung cancer tissue type, and identifies samples by an ID tag. Table 1B provides the matrix location of the protein samples used. Antibody reactivity with protein samples is monitored as described in FIGS. 1A to 1D. The schematic diagram below the figure provides in 96 table format (rows A-H, columns 1-12) the ID No of the antibodies used in the experiment, and their location in the matrix protein array.

FIG. 3: Polynucleotide listing.

The nucleotide sequence of a selected group of polynucleotides of the present invention is listed here, with the ID No of the corresponding antibody, and the accession number from the GenBank database. Using the accession number and antibody ID No., the SEQ. ID No. for the specific gene sequence and polypeptide sequence corresponding to the antibody can be determined by referring to Tables 3 and 4 and the sequence listing submitted herewith.

FIG. 4: Polypeptide listing.

The amino acid sequence of a selected group of polypeptides of the present invention is listed here in one-letter amino acid code, with the ID No of the corresponding antibody, and the accession number of the corresponding polynucleotide sequence (listed in FIG. 1). Polynucleotide-antibody correspondence can be further found in Table 4.

Table 1A: Clinical information on lung specimens.

This table summarizes clinical information on normal and diseased tissue and plasma samples from lung cancer patients used in FIGS. 1A to 1D and FIG. 2. “Sample ID” refers to a numbering system used to locate samples on the matrix protein array experiments shown in FIGS. 1 to 5; “source” refers to tissue or plasma; “type” refers to the histological type of lung cancer from the patient's anonymous pathology reports (i.e. normal, adenocarcinoma, squamous cell carcinoma, bronchioalveolar); “stage” indicates the stage of lung cancer for that sample, according to the patient's pathology report and the staging classification of the American Joint Committee on Cancer (in: AJCC Cancer Staging Manual, Philadelphia, Pa.: Lippincott-Raven, 5^{th} Ed, 1997); “protein set” provides easy reference to the relevant figure and matrix protein array experiment where those specimens have been used.

Table 1B: Human lung specimen location in matrix protein array experiments.

Schematic diagram of the location of the tissue and plasma samples described in Table 1A in the matrix protein array experiments shown in FIGS. 1A to 1D and FIG. 2. “n” refers to normal samples; “da”, “db”, “ds” respectively refer to adenocarcinoma, bronchioalveolar carcinoma, and squamous carcinoma of the lung (diseased samples).

Table 2: Data analysis of lung cancer specific antibodies and their reactivity in lung cancer tissues.

This table summarizes the reactivity of 83 antibodies of the present invention identified by their ID No, on lung tumor tissues derived from different lung cancer types (see Table 1A). The column “reactive samples” indicate those samples, identified by their ID No (see Table 1B), that display differential antibody reactivity (either up or down, as indicated in the column “regulation”) with respect to normal. N is the number of matched spots considered for the analysis. Prevalence, ratio of disease versus normal, and N are indicated and are as defined in the specifications.

Table 3: Data analysis of lung cancer specific antibodies and their reactivity in plasma of lung cancer patients.

This table summarizes the reactivity of 96 antibodies of the present invention identified by their ID No, on plasma from lung cancer patients (see Table 1A). Same legend as for Table 2, above. N is the number of diseased spots.

Table 4: Lung cancer related polynucleotide sequences and antibodies.

This table lists the ID No of the 159 lung cancer related antibodies of the present invention, and the accession numbers of the associated polynucleotide sequences. GenBank accession numbers of polynucleotide sequences from the public database sharing homology with the polynucleotide sequences of the present invention were obtained through BLAST searches. Top 10 BLAST results are indicated.

### DETAILED DESCRIPTION OF THE INVENTION

The present invention pertains to the disclosure of gene products correlating with the presence of lung cancer, their DNA (RNA) sequence, their polynucleotide and peptide derivatives, and their antibodies. Furthermore, the present invention relates to the methods of use of the above mentioned molecules in diagnostic and therapeutic applications.

Polynucleotides

In accordance with one aspect of the present invention, there is provided isolated polynucleotide sequences which encode gene products whose expression levels is altered in human lung cancer.

The term “polynucleotide”, “nucleotide sequence”, “nucleic acid molecules” are used interchangeably herein and may include DNA or RNA. Sequence information and homology searches have been determined for the polynucleotides of the present invention. BLAST is a preferred example among algorithms that are suitable for determining percent sequence identity and sequence homology for the polynucleotides and polypeptides of the invention (Altschul, S. F., Gish, W., Miller, W., Myers, E. W. & Lipman, D. J. (1990) “Basic local alignment search tool.” J. Mol. Biol. 215:403-410). Software for performing BLAST analyses is publicly available through the National Center for Biotechnology Information (NCBI). Table 4 provides information on the polynucleotide sequences of the present invention: specifically, it provides the GenBank accession number of the top one to top ten homologues (with the highest scores) to the sequences of the present invention, as obtained by BLAST search of the public database. Table 4 also provides the ID No of the lung cancer related antibodies of the present invention, which are associated with those sequences. FIG. 3 provides the nucleotide sequences of a selected group of polynucleotides of the present invention.

The polynucleotides described herein may be in the form of DNA, including cDNA, genomic and synthetic DNA, double or single-stranded, coding or non-coding strand. The polynucleotides encompassed by the appended claims may be in the form of RNA, including heteronuclear RNA, messenger RNA, or any other form of RNA, such as small, anti-sense, interfering or silencing RNA. The polynucleotides may or may not contain introns, 5′ and 3′ non coding sequences, 5′ and 3′ transcriptional regulatory sequences, such as promoters, enhancers, or polyadenylation signals, translational control elements. The polynucleotides may or may not encode for a leader or secretory sequences at the level of the polypeptide, or for an active or inactive pro-protein that is later processed into active or inactive shorter polypeptides.

The polynucleotides herein may include variants of said polynucleotides, which may be naturally-occurring allelic variants or non-naturally-occurring variants, and may include variants which encode the gene products of the present invention with a different nucleotide sequence due to the degeneracy of the genetic code. Variants may encode fragments, analogs and derivatives of the polypeptides encompassed by the present invention, and may include deletion, substitution, addition or insertion variants. The polynucleotides encompassed by the appended claims include any length of said polynucleotide sequence, whether 5′ terminal, 3′ terminal or internal.

Nucleic acid molecules of the present invention can be isolated using standard molecular biology techniques and the sequence information described herein. Using all or portions of such nucleic acid sequences, nucleic acid molecules of the invention can be isolated using for example standard oligonucleotide synthesis, PCR and hybridization and cloning techniques (e.g. as described in Sambrook et al., 1989, Molecular Cloning: A Laboratory Manual, 2^{nd} ed., Cold Spring Harbor Laboratory Press, Cold Spring Harbor, N.Y.). Using the techniques mentioned above and known to the skilled in the art, the polynucleotides of this invention can be obtained from human cDNA libraries from a variety of tissues and organs, from normal and diseased state, and various stages of disease, from substractive cDNA libraries, SAGE or DNA microarray analysis (Diatchenko et al., 1996, PNAS 93:6025-6030; Velculescu V E et al., 1995, Science 270:484-487; van't Veer et al., Gene expression profiling predicts clinical outcome of breast cancer, Nature (2002) 415:530-535).

Polypeptides

The term of “polypeptide”, “peptide”, “gene product” are interchangeably used to indicate an amino acid sequence resulting or deduced from a nucleotide sequence according to the amino acid triplet correspondence as defined per the genetic code. The term “genetic code” is used herein to include unorthodox genetic codes as used in a variety of species (such as Paramecium and Tetrahymena), or sub-cellular organelles (e.g.mitochondria) or encoded by minor tRNA species.

In accordance with one aspect of the present invention, there are provided a multiplicity of polypeptide sequences of human origin, as well as fragments, analogs and derivatives thereof. The polypeptides referred to herein represent a population of polypeptides that can be encoded by the lung cancer related polynucleotides identified in Table 4. FIG. 4 provides the amino acid sequences of a selected group of polynucleotides of the present invention.

Said translation may occur from one strand, or its complementary sequence, may include so-called non-coding and regulatory sequences, some of which are listed above, and may occur in all possible reading frames. Indeed, any nucleotide sequence, harboring the appropriate gene expression determinants, may give rise to several transcripts of different lengths, displaying different open reading frames with different start and stop codons. This in turn results in the translation of different polypeptides which could be either isoform or variant polypeptides to each other (e.g. fragment, and analogs) or distinct polypeptides.

The population of polypeptides of the present invention includes those generated from the polynucleotide sequences claimed herein by unconventional mechanisms, such as but not restricted to frameshift at the nucleotide sequence level (e.g. insertion, deletion, or substitution variants mentioned above) or occasional or programmed frameshift, internal initiation, or non Watson-Crick codon-anticodon pairing events at the translation level. These and other mechanisms may lead to the production of hybrid polypeptides, for example carrying amino acid motifs of one reading frame and/or amino acid motifs expressed from another reading frame. Such hybrid protein carrying multiple amino acid domains may consequently be regulated according to as many different -functional domains as featured in the hybrid polypeptide. Hybrid polypeptides may retain substantially the same biological function or activity as the relevant lung cancer gene products while partially differing in the amino acid sequence.

Polypeptides encompassed by the present invention may include fusion to a marker sequence supplied by an expression vector and enabling purification of the polypeptide of the present invention, such as hexa-histidine tag, glutathione-S-transferase, hematgglutinin, luciferase, beta-galactosidase, and the like.

The polypeptides of the present invention additionally include modified polypeptides, in full or in part, by any form of post-translational modification, such as phosphorylation, acylation, methylation, ubiquitination, etc., conjugation or covalent linkage to lipids, polysaccharides and the like.

The polypeptides of the present invention further include full-length mature folded proteins, or fragments thereof, either derived by internal initiation, early termination, degradation, or post-translational processing.

The polypeptides of the present invention may be a natural polypeptide, recombinant polypeptide as generated below, or a synthetic polypeptide. The polypeptides of the present invention may be provided in an isolated form, preferably purified to homogeneity, or linked to a tag for purification purposes, or may be present in a biological mixture, such as that of a protein extract from human specimens.

In addition to recombinant production methods, polypeptides of the invention, and fragments thereof, including relevant isolated antigenic determinants and structural protein domains may be produced by direct peptide synthesis using solid-phase techniques (Merrifield, 1963). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Expression Systems

The polynucleotides of the present invention may be employed for producing polypeptides by recombinant techniques. To this end, the present invention also relates to nucleotide sequence cassettes and related methodology to modify a plasmid into a suitable expression vector designed for the expression of polynucleotide sequences of the present invention in bacterial and mammalian systems, and for the production and purification of the corresponding gene products and polypeptides of the present invention by recombinant techniques in bacterial cells.

As known to the skilled in the art, a eukaryotic expression vector preferably includes a promoter nucleotide sequence operably linked to the sequence encoding the polypeptides of interest, such as the cytomegalovirus (CMV), simian virus 40 (SV40), phosphoglycerate kinase (PGK), or actin promoters, or any other tissue specific promoters, with or without additional appropriate regulatory elements to achieve the highest expression possible of the cloned polynucleotide sequence. Examples include enhancers and Long Terminal Repeat sequences (LTRs) for expression in eukaryotic cells. In addition, it is preferred that downstream of the sequence of interest, the vector features a polyadenylation sequence signaling termination of transcription, such as those derived from the CMV, the bovine (BGH) and human growth hormone (HGH) or the SV40 large T antigen genes. Whether for prokaryotic or eukaryotic expression, high copy number vectors are generally preferred.

In view of bacterial expression, it is known to the skilled in the art that the sequence to be expressed is preferably driven by a strong prokaryotic promoter such as lacI, lacZ, trp, LamB, gpt, bacteriophage T3, T7, lambda, P_{R}, P_{L}. The relevant sequence is also operably linked to a ribosome binding sites for initiation of translation.

In addition, the expression vector preferably contains a resistance phenotypic trait for selection of transformed host cells such as a neomycin resistance gene for eukaryotic cells, or tetracyclin, ampicillin or chloramphenicol for resistance in bacterial cells.

Other sequences, such as polylinkers, origins of replication, whether functional in bacterial and/or eukaryotic cells, bacterial selection markers, reporter genes, as well as tags designed for protein purification, are preferably included to facilitate examination of expression as known by the skilled in the art, such as hexa-histidine tag, glutathione-S-transferase, hematgglutinin, luciferase, beta-galactosidase, and the like.

In particular, eukaryotic origin of replication and integration sequence for maintaining the expression of the sequence of interest for an extended period of time may be preferred for expression in mammalian cells.

Several plasmid constructs containing the preferred constituents described above, some of them, or their equivalents, are commercially available from sources such as Novagen, Amersham, Invitrogen, Stratagene and others. Also, a variety of expression vectors for expressing polypeptides can be designed, containing chromosomal or non-chromosomal DNA sequence, phage DNA, viral DNA such as vaccinia, adenovirus, Sindbis virus or the like. Any other vector may be used as long as it is replicable and viable in a host of choice.

Preferably, the host for expression and purification is bacterial cells, such as E.coli. Various mammalian cell culture systems can be employed to express recombinant proteins, such as COS-7, HeLa, monkey kidney cells, CHO, BHK. Alternatively, host such as insect, fungi or plant cells may be used to express the relevant gene products of the invention, provided the appropriate expression elements are provided in the expression vector.

The polynucleotides of interest may be inserted in the selected expression vectors by methods and procedures known in the art. For the purpose of the present invention a “cassette” strategy, described in the Example 7 herein, was designed. The cassette used for bacterial expression carries a kanamycin resistance gene, and two sets of functional transcription and translation units in opposite orientations, one under the control of the bacteriophage T7 promoter, and another under the control of the hybrid trp-lac promoter. Each translation unit comprises a ribosome binding site, an initiator ATG, and a His6 tag for protein purification. For the purpose of the present invention the cassette used for mammalian expression carries an SV40 and a CMV promoters in opposite orientation and ampicillin resistance. Whether in view of bacterial or mammalian expression system, the cassette is ligated to the target plasmid containing the polynucleotide of interest, previously linearized with restriction enzymes allowing compatible ends with the cassette. The target polynucleotide can be expressed in either orientation, from the transcription and translation elements provided by the cassette sequence. Both vector and cassette exist under three possible configurations to accommodate expression in all possible reading frames.

As an alternate method to expression and purification, the polypeptides of the present invention can be generated by high-throughput cell-free translation systems (Sawasaki et al., 2002) of RNA transcript molecules synthesized by run-off in vitro transcription from T7 or Sp6 bacteriophage promoter vectors, as known in the art.

Antibodies

As used herein the term “antibody” refers to a polyclonal, monoclonal or recombinant antibody having specific binding affinity to a peptide, a polypeptide, or a gene product, or to fragments and derivatives thereof, from the population of polypeptides encompassed by the present invention. The term “antibody” will be used to indicate polyclonal, monoclonal or recombinant interchangeably, unless specifically indicated such as in the Examples herein.

Polyclonal antibodies form an heterogeneous population of antibody molecules. Polyclonals are generated by the immune system of an animal, immunized with an antigen (i.e. a cancer specific polypeptide) or an antigenic functional derivative thereof (i.e. a selected amino acid sequence of the relevant polypeptide), as well known by the person of ordinary skills in the immunological arts (Colligan et al., Current Protocoles in Immunology, Wiley Interscience).

Monoclonals antibodies may be obtained by any technique that leads to the production of antibody molecules by continuous cell lines in culture. Said methods are known to those skilled in the art (Kohler, et al., Nature 256:495-497 (1975), and U.S. Pat. No. 4,376,110). Recombinant antibodies may be generated in vitro, by methods known in the art (“Phage display of peptides and proteins—A Laboratory Manual, Kay B. B., Winter J. & McCafferty J, Eds. Academic Press, 1996).

Monoclonal and recombinant antibodies are meant to include intact full-size molecules or antibody fragments, preferably Fab and scFv (single chain variable Fragment) capable of binding to an epitope expressed by one or more of the polypeptides of the present invention. Furthermore, monoclonal and recombinant antibodies are monospecific, i.e. are a substantially homogenous populations of antibodies to a particular antigen.

The polyclonal antibodies disclosed herein react with antigenic determinants that are differentially expressed in protein samples derived from cancer patients as compared to normal controls (see section “Differential expression of lung cancer related gene products” below). Preferably said polyclonals are of mouse origin, however they may be generated in any other mammal capable of immune response. The lung cancer related antibodies of the present invention are 159 antibodies, identified throughout by their ID No. Correspondence between antibodies and polynucleotide sequence of the present invention is provided in Table 4.

The reactivity of the lung cancer related antibodies may be directed at the polypeptides of the present invention encoded by the polynucleotides of the present invention. Because of their polyclonal nature, antibodies of the present invention may be immunologically reactive against one or more polypeptides described herein. Furthermore, antibodies of the present invention may be reactive against proteins not described herein that yet share substantially similar antigenic determinants or structural domains with the polypeptides that have elicited the generation of said antibodies. Indeed the following is known to the skilled in the art: (a) the existence of protein families sharing amino acid sequence and functional homology is well-established, thus different polypeptides of the present invention may share common amino acid motifs or functional domains with other polypeptides present in patient's protein samples, (b) polypeptide variants can be expressed and purified from a single polynucleotide sequence giving rise to polyclonal antibodies against a population of polypeptide variants, (c) amino acid motifs with regulatory function in the cell, may be common to a variety of proteins that are elicited in diseased or cancerous state.

Differential Expression of Lung Cancer Related Gene Products

Antibodies of the present invention can be used to examine the differential or relative expression of the corresponding gene products or structural domains of the present invention in normal versus diseased human specimens, such as tissues and biological fluids. The term “antigenic determinant”, “structural domain”, “antibody target” are interchangeably used to indicate the amino acid sequence, whether in isolated form or embedded in a polypeptide sequence or fragment and derivative thereof, that is recognized by the antibodies of the present invention and associated to cancer.

For the purpose of the invention and for most diagnostic and therapeutic applications, biological samples referred herein are from human patients and normal controls, and may include fresh or frozen normal and diseased tissues, such as derived from a tumor biopsy, or biological fluids such as ascites, urine, plasma, serum, blood, saliva, sputum, any lavages such as but not restricted to ductal lavages, bronchio-alveolar ravages, any other fluid, such as, but not restricted to spinal or cerebral fluid, nipple aspirate fluid, or any other preparation that may be processed for advantageous use in the kits of the invention. For the purpose of the invention protein samples can also be derived from mammalian cell cultures, microdissected cell types from normal or diseased tissue samples, or from a given subcellular compartment. However the methodology described herein is applicable as well to protein samples derived from any organismal source, such as bacterium, yeast, fungus, or plant.

In the preferred embodiments of the present invention, the biological samples examined may be matched normal and tumor tissues (i.e. derived from the same patient), or normal and tumor tissues derived from different lung cancer patients. Samples may include primary tumor or metastasis, benign tumors, any stage of lung cancer from stage I (early) to stage IV (late), and any histologic type or subtype of lung cancer. Table 1A lists clinically relevant information about the specimens used herein. Preparation of protein samples from normal and tumor specimens is described in Example 2.

Many techniques, such as DNA microarrays, SAGE, differential display, are available to detect differential gene expression at the RNA level. Proteomic approaches such as comparative use of two-dimensional electrophoresis coupled to mass spectroscopy analyses may generate two-dimensional profiles between normal and diseased samples (Emmert-Buck M R et al., 2000, Mol Carcinog 27:158-165). Serum protein expression profiles between normal and cancer samples may be obtained through SELDI-TOF-MS (surface enhanced laser desorption/ionization time of flight mass spectroscopy; Petricoin E F et al., 2002, Nat Rev Drug Disc 1:683-695), yet this technology provides no direct information on the identity of the protein.

The methodology used by the applicants to assess differential expression of gene products of the present invention is by means of immunodetection, using the antibodies of the present invention and a process namely the matrix protein array as described in Example 3. A unique feature of the present invention is to enable the examination of the relative expression levels of proteins of interest in multiplex format. By “multiplex format”, it is herein referred to the analysis of a large number of proteins of interest, with a large number of identifiers and in a large number of biological samples simultaneously. The term “identifier” refers to a molecule that binds specifically to its target in a sample. Preferably the identifier is an antibody (polyclonal, monoclonal, Fab fragment, single chain, affibody, or any other recombinant version of conventional or combinatorial antibodies), specifically recognizing a protein or structural domain, or the identifier is a peptide, a mimotope, a ligand, substrate, carbohydrate, lipid, drug compound, or any other organic molecule or biological tag.

The solid support of the matrix protein array is preferably nitrocellulose or glass, yet it can be made of a variety of materials that include, but are not limited to: plastic, polystyrene, nylon, teflon, ceramic, fiber optic and semiconductor materials. The solid support of the matrix protein array is composed of different physical areas that can be referred to as wells, compartments, surfaces, and the like, distinctly separated from each other. These physical areas can adopt a variety of surfaces and volumes, and the support can accommodate from 1 or 2 to several hundreds of compartments, depending on the needs, leading to an extremely versatile tool. This is achieved by having the device composed of a base and a divider, the format of the latter determining the size and number of the compartments. Each compartment may contain biological samples from the same type, different types, the same species, different species, the same physiological conditions, different physiological conditions or any combination of the above arrayed on the solid support. Each compartment is overlayed with any identifier, as selected. It is understood by those skilled in the art that the device described as matrix protein array in the present invention allows all kind of combination of biological samples, number of samples, conditions of the samples, size of compartment of the matrix protein arrays, type of identifiers, or any permutation of the above.

In the immunodetection analysis detailed in Example 3 and described in a number of preferred embodiments of the present invention, the detection of relevant gene products or targets identified within a complex biological mixture (i.e. antibody-antigen complexes) is preferably performed by way of a chemiluminescent reaction, although protocols based on other labeling and detection systems, such as alkaline-phosphatase, biotin-streptavidine, or fluorescence systems can also be successfully used within the scope of the present invention. Antigen-antibody or target-identifier signals are captured by a charge-coupled device (CCD) camera, processed and quantified by a specialized software, and data analyzed as described in Example 5 and 6.

In a preferred embodiment of the present invention (FIGS. 1A to 1D) antibodies of the present invention are reacted on lung cancer specimens by means of the matrix protein array analysis. Antibodies are used to examine the differential expression of their corresponding gene products or structural domains (i.e. antibody target) on matched normal and tumor tissues from lung cancer patients, using the matrix protein array analysis (described above and in Example 3). Table 1A and 1B lists clinically relevant information on normal and lung cancer specimens used herein. Preparation of protein samples from tissues of lung cancer patients is described in Example 2. As evidenced in FIGS. 1A to 1D, antibodies react with overexpressed or underexpressed targets in lung cancer protein samples versus normal controls. In another preferred embodiment of the present invention (FIG. 2) antibodies of the present invention are used to examine the differential expression of their corresponding gene products or structural domains (i.e. antibody target) on plasma samples from lung cancer patients, using the matrix protein array analysis (described above and in Example 3). Table 1A and 1B lists clinically relevant information on normal and lung cancer specimens used herein. Preparation of protein samples from plasma samples of lung cancer patients is described in Example 2. As evidenced in FIG. 2, antibodies react with overexpressed or underexpressed targets in lung cancer versus normal controls.

It is a further embodiment of the present invention that said antibodies enable detection of lung cancer tissue targets at early stage of lung cancer and in a variety of histologic tumor types, including but not restricted to the non small cell lung carcinoma, the most prevalent lung tumor types (see Table 1A).

A “lung cancer structural domain”, “lung cancer antigenic determinant” or “lung cancer target” is herein referred to an amino acid sequence, whether in isolated form or embedded in a polypeptide or fragment and derivative thereof, which is recognized by antibodies of the present invention, and that is differentially expressed in lung cancer plasma or tissues versus normal controls.

Table 2 and 3 provide a summary of the reactivity of the lung cancer related antibodies of the present invention on lung cancer tissues and plasma, respectively. Table 2 summarizes the reactivity of 83 antibodies of the present invention on lung cancer tissues. Prevalence of the antibody targets among the lung cancer patients is defined as the percentage of sample spots in one class (i.e. diseased spots for up-regulated targets; or normal spots for down-regulated targets) that are at least 50% brighter than the corresponding matched sample spot in the other class (i.e. normal spots for up-regulated targets; or diseased spots for down-regulated targets). Ratio D/N is defined as the average of the intensity ratio between each matched diseased and normal spots (ratio D/N=Σ[D1/N1+D2/N2+ . . . Dn/Nn)]/n, where D indicates a diseased spot intensity, N indicates a normal spot intensity, and n the number of matched spots samples considered for the calculation. For duplicate samples (such as in the case of protein set 5), intensity of duplicate spots are averaged prior to the calculation. Up-regulated targets have D/N ratio>1, while down-regulated targets have D/N ratio<1.

Table 3 summarizes the reactivity of 96 antibodies of the present invention in lung cancer plasma. Raw data analysis in the case of non-matched samples (protein set 6) is modified as follows. Prevalence is defined as the percentage of diseased sample spots that are 50% brighter (for up-regulated targets) or 50% darker (for down-regulated targets) than the average intensity of normal sample spots. Ratio D/N represents the average intensity of diseased sample spots divided by the average intensity of normal sample spots.

Statistical analysis may then be performed as described in Example 5 and 6.

In conclusion, the present invention provides evidence for the specific overexpression or underexpression of the gene products, structural domains, or antigenic determinants of the present invention in relation to lung cancer. Therefore the present invention provides evidence for the function of one or all such gene products as markers for lung cancer detection, progression and disease management, whether those gene products are present in tissues or secreted in biological fluids. The present invention also provides a method for utilizing the lung cancer related antibodies as diagnostic tools. Example 6 provides such method whereby one or a panel of antibodies that recognize cancer specific targets can be used to detect lung cancer in an unknown population of patients.

It is within the scope of the present invention the application of antibodies and matrix protein array to other biological specimens derived from lung cancer patients and normal controls, including serum, bronchioalveolar lavages, sputum and the like, or to tissue samples derived from any other histological type or subtype of lung cancer, and any stage of lung cancer.

In another embodiment of the present invention the differential reactivity of antibodies of the present invention may be examined in a variety of human cancer cell lines by use of the matrix protein array to further substantiate the disease relatedness of the gene products and antibodies of the present invention.

To evaluate the potential use of the antibody targets of the present invention in therapeutic applications, the reactivity of the antibodies in a variety of normal tissues may be further examined using the matrix protein array methodology. Hence, in a preferred embodiment of the present invention protein extracts from different normal tissues may be printed on the matrix protein array and reacted with a given antibody, as described in Example 3 and 4. Normal tissues may include, but are not restricted to: colon, heart, liver, lung, intestine, kidney, muscle, pancreas, spleen, stomach, testis, ovary, brain. Antibodies reacting with antigenic determinants that are present in lung cancer patients and have little or no expression in normal tissues, identify targets that are particularly suitable for therapeutic purposes. It is indeed anticipated that antibodies against such targets or antigenic determinants thereof, will cause little adverse secondary effects to normal tissues when used to treat cancer in the context of antibody or protein therapeutics.

Further Uses of Lung Cancer Specific Antibodies

Antibodies of the present invention can be used to further explore the function and further characterize the corresponding lung cancer related proteins or antigenic determinants. For example, the specificity of the lung cancer related antibodies may be analyzed by Western blot on protein extracts from tissues or relevant mammalian cancer cell lines, or on biological fluids. The apparent molecular weight of the antigen recognized by said antibodies may be determined.

The antibodies of the present invention have great utility in the further characterization of gene products of the present invention, as provided in a further embodiment herein. Antibodies of the present invention can be used in multiple studies using a variety of biochemical techniques and cellular biology assays known to those skilled in the art to further elucidate the function of the relevant gene products. A particular example is the cellular localization of the lung cancer specific gene products. In mammals, gene products can be either secreted outside of the cell such as growth factors, hormones, neuropeptides, can be present on the cell surface such as proteins, glycoproteins, glycolopids and receptors, or can occur inside the cell, either within the cytosol, or in particular sub-cell compartments, such as the nucleus, the Golgi, or the endoplasmic reticulum. These gene products can be localized to cellular structures via the use of their corresponding antibodies. When gene products are localized to the cell surface, they may be potentially blocked in their functions by a specific antibody or fragment thereof. If the lung specific gene product or determinant is overexpressed in disease state, the use of an agonist antibody may have therapeutic effect.

A variety of techniques known to those skilled in the art can be used to confirm cellular localization of cancer related gene products. A preferred method is based on immunostaining using peroxidase linked secondary antibodies against primary antibodies. Immunostaining can be carried out to cells grown in individual chamber slides or 6-well dishes, as well as to cells grown in 96-well culture plates or more for high-throughput localization studies. Specific staining of gene products by the corresponding antibody can be localized to nuclei, cell membrane or cytosol. Antibodies reacting with the cell surface identify targets that have utility in therapeutic applications.

Antibodies against relevant gene products can be used as well for localization to further subcellular structures by electron microscopy and other imaging techniques. A variety of methods such as immunfluorscence (IF) using FACScan (FACS), flow cytometry (FC) and indirect IF, known to those in the art, can be performed on mammalian cell suspension or adherent cells, as described in (Current Protocols in Immunology, Wiley Interscience, John E. Colligan et al.). Alternatively, antibodies can be used to determine the presence or absence of the cancer specific gene products in fixed tissue microarrays from normal and diseased cancer specimens.

Antibodies of the present invention can be used in multiple studies using a variety of biochemical techniques and cellular biology assays known to those skilled in the art to elucidate the function of the relevant gene products associated to lung cancer. Antibodies of the present invention can be used to purify large amounts of cancer specific gene products for further studies, including structure determination by mass spectrometry. Specifically, such antibodies are used for the affinity purification of said proteins from recombinant cell culture (see above) or natural sources. In this process, antibodies of the present invention are immobilized on a suitable support, such a Sephadex resin or filter paper, using methods known in the art. The immobilized antibody is then contacted with a sample containing the cancer related protein to be purified, and thereafter the support is washed with a suitable solvent that will remove substantially all the material in the sample except the relevant protein, which is bound to the immobilized antibody. Finally, the support is washed with another suitable solvent that will release the relevant protein from the antibody.

To analyze differential gene expression of lung cancer related gene products in cellular pathways in response to external stimuli, normal and diseased biological samples may be analyzed with the antibodies described herein and by matrix protein array. For example the gene expression of various cell types and biological samples can be analyzed following exposure to hormones, growth factors, bioactive chemicals generally drugs, especially chemotherapy compounds, and virtually any toxin or agent whose effect on cell growth or metabolism and the underlying gene expression is of interest. Indeed the use of antibodies enables gene product expression profiling in a wide variety of conditions enabling the discovery of the biological activity and function of the relevant lung cancer gene products in relation to cellular pathways and networks, and virtually in any pathological and physiological condition characteristic of a normal or disease state. The antibodies against lung cancer related gene products can be further used to investigate the alteration of expression of such gene products in cellular assays, preferably in cell cultures of mammalian origin, in the presence or absence of a variety of biologically active compounds, such as hormones, or any other effector of cell growth and metabolism. Antibodies against lung cancer gene products can be further used to assay a variety of normal and cancerous cell lines before and after exposure to drugs, including anti-cancer drugs, and pharmacological agents, as well as to examine toxicity profiles. Such pharmacoproteomic application can be extended to chemicals, physical stimuli, such as carcinogens, irradiation, toxic compounds, and the like.

Antibodies of the present invention can be used to detect lung cancer specific gene products and their expression levels in animal models of cancer. This provides further correlation between the gene product and the disease process under investigation, as well as evidence for the utility of that cancer specific gene product in therapeutic applications.

Diagnostic Uses of Antibodies

It has been demonstrated above that antibodies of the present invention may be used for diagnostic purposes. Indeed, antibodies which specifically bind to cancer specific proteins may be used for the diagnosis of cancer and other diseases characterized by altered expression of said proteins, or in assays to monitor patients being treated for said conditions, with agonists, antagonists or inhibitors of those cancer specific proteins. Hence, antibodies against lung cancer specific targets may be used for lung cancer detection and diagnosis.

Such diagnostic assays utilize one or a panel of the lung cancer specific antibodies of the present invention, and a label to detect lung cancer specific proteins in human body fluids, cell or tissue extracts, as embodied in Examples 3 and 5. Other diagnostic techniques such as competitive binding assays, direct or indirect sandwich assays and immunoprecipitation assays can be used [Zola H., Monoclonal Antibodies: A Manual of Techniques, CRC Press, Inc. (1987) pp. 147-158]. Antibodies used in diagnostic assays need to be labeled with a detectable moiety, which can be a radioisotope, a fluorescent or chemiluminescent compound used in conjunction with an enzyme-linked antibody. Examples of radioisotope label are: 3H, 14C, 32P, 35S, or 125I. Examples of suitable enzyme labels for use in ELISA-type systems preferably include horseradish peroxidase and alkaline phosphatase as described in the Examples herein. Other enzyme labels include: glucose oxidase, catalase, glucose-6-phosphate dehydrogenase, acetylcholinesterase, or beta-galactosidase. Examples of chemiluminescent labels preferably include luminol label reacting with horseradish peroxidase, but may also include: luciferin reacting with luciferase, aequorin, or CDP-star and CSPD (1,2,-dioxetane derivatives), which are substrates for alkaline phosphatase. Examples of suitable fluorescent labels include fluorescein label, rhodamine label, oregon green, coumarin, texas-red, phycoerythrin.

The antibodies disclosed in the present invention may be used in diagnostic in vivo imaging to detect tumors. Examples of suitable radioisotopic labels include H, 111In, 125I, 131I, 32P, 35S, 14C, 51Cr, 57To, 58Co, 59Fe, 75Se, 152Eu, 90Y, 67Cu, 217Ci, 211At, 212Pb, 47Sc, 109Pd, etc. 111In is a preferred isotope where the antibodies are to be applied for in vivo imaging, as this radionucleotide has a more favorable gamma emission energy for imaging (Perkins et al., 1985, Eur. J. Nucl. Med. 10:296-301). Examples of nuclear magnetic resonance contrasting agents, which are also useful in medical imaging applications, include heavy metal nuclei such as Gd, Mn, and Fe. Typical techniques for binding the above-described labels to antibodies are provided by Kennedy et al. (Clin. Chim. Acta 70:1-31 (1976).

Therapeutic Uses of Polypeptides and Antibodies of the Present Invention

The term “agonist”, as used herein, refers to a molecule which, when bound to a protein, causes a change in said protein, modulating the activity of said protein. Agonists may include proteins, nucleic acids, carbohydrates, or any other molecules which bind to said membrane proteins. The terms “antagonist” or “inhibitor”, as used herein, refer to a molecule which, when bound to a protein, blocks or modulates the biological or immunological activity of said protein. Antagonists and inhibitors may include proteins, nucleic acids, carbohydrates, or any other molecules which bind to said proteins, including antibodies.

Antagonists or inhibitors of cancer specific proteins of the present invention, preferably including the lung cancer specific antibodies of the present invention, may be administered to a subject to treat or prevent lung cancer. Specific antibodies may be used directly as an antagonist, or indirectly as a targeting or delivery mechanism for bringing a pharmaceutical agent to cells or tissues which overexpress lung cancer specific proteins of the present invention. Lung cancer as well as other diseases associated to the lung cancer targets of the present invention, may be thus treated by immunotherapy, by administration or delivery of antibodies of the present invention.

The antibodies disclosed in the present invention may also be “humanized” for use as therapeutic compounds in human beings. Humanization of antibodies may be necessary in order to prevent detrimental immunological reactions against animal-produced antibodies when they are administered to people. Methods for producing humanized antibodies are known in the art (Morrisson, Science 229:1202, 1985).

The use of the lung cancer specific targets of the present invention in protein therapeutics is contemplated herein, particularly if said polypeptides are found underexpressed in cancer, and have low or no expression in normal tissues other than the relevant cancer tissue. Administration of said protein to a cell culture or a subject for ex vivo or in vivo therapy may be achieved via expression vectors.

Expression vectors derived from retroviruses, adenovirus, herpes or vaccinia viruses, or from various bacterial plasmids may be used for delivery of nucleotide sequences (including for expression of relevant proteins for therapeutic purposes, see above) to the targeted organ, tissue or cell population. Many methods for introducing vectors into cells or tissues are available and equally suitable for use in vivo, in vitro, and ex vivo, including delivery by transfection, liposomes, introduction into stem cells taken from the patient and clonally propagated for autologous transplant back into that same patient.

Alternative therapeutic methods that do not use proteins or antibodies, but use polynucleotides encoding such proteins or variants thereof, are contemplated herein. For example, a vector expressing antisense molecule of the polynucleotide encoding lung cancer specific proteins may be administered to a subject to treat or prevent disorders which are associated with dysregulated expression of said proteins. Such disorders include but are not limited to lung cancer, as demonstrated in the attached embodiments, or include other disorders related to abnormal cellular differentiation, proliferation or degeneration. Antisense sequences can be used to block or regulate transcription and translation, and thus expression of relevant proteins, as well known now in the art.

In addition to antisense sequences, inhibition of expression can be achieved using “triple helix” base-pairing methodology (Gee, J. E. et al., 1994, in: Huber, B. E. and B. I. Carr, Molecular and Immunologic Approaches, Futura Publishing Co., Mt. Kisco, N.Y.), ribozymes, enzymatic RNA molecules, that specifically catalyze the endonucleolytic cleavage of RNA encoding polypeptides of the present invention, or RNA interference approach that enables the specific silencing of gene expression in a custom-based manner (Paul, C. P. et al., 2002).

Any of the therapeutic proteins, antagonists, antibodies, agonists, antisense sequences or vectors described above may be administered in combination with other appropriate therapeutic agents. Such combination therapies may achieve improved therapeutic efficacy with lower potential for adverse side effects. Any of the therapeutic agent discussed above may be administered as a pharmaceutical composition in conjunction with a pharmaceutically acceptable carrier, by any number of routes.

Purified proteins of the present invention may be used to further produce larger amounts of antibodies or to screen libraries of pharmaceutical agents such as small molecules, to identify those which specifically bind said proteins.

These and other aspects of the present invention should be apparent to those of skill in the arts from the teachings herein. The following examples are offered below to illustrate particular embodiments of the invention, and should not be interpreted as to so limit the claimed composition and uses. Given the examples and instruction afforded by the specifications, variants of the claimed composition and uses not enumerated herein will also be apparent to those of skill in the art, and are contemplated as within the scope of the invention. The following figures are illustrative of embodiments of the inventions and are not meant to limit the scope of the invention as encompassed by the claims.

### EXAMPLES

The following abbreviations will be used throughout. h: hour; min: minutes; sec: seconds; rpm: rotation per minutes; RT: room temperature.

### Example 1

Polynucleotide Sequences

Polynucleotides encompassed by this invention are listed in Table 4. Table 4 provides the GenBank accession number of the top one to top ten polynucieotide sequences found by BLAST searches of the public database, and sharing homology with the polynucleotides described herein. Table 4 also provides the ID No of the 159 lung cancer related antibodies of the present invention, which are associated with those polynucleotide sequences.

DNA sequences of the present invention can be isolated using standard molecular cloning, hybridization and PCR techniques (e.g. described in Sambrook et al., 1989, Molecular Cloning: A laboratory Manual, 2^{nd} Ed., Cold Spring Harbor Laboratory Press, Cold Spring Harbor, N.Y.) together with the sequence information provided in Table 5. DNA clones can be prepared for sequencing via conventional alkaline lysis method.

DNA sequencing is performed using an ABI 377 automated DNA sequencer (Applied Biosystems, Foster City, Calif.) and a fluorescent dye terminator sequencing reaction protocol, either Big Dye (ABI, Foster City, Calif.) or Dynamic ET (Amersham Pharmacia Biotech, Piscataway, N.J.), according to the manufacturer's instructions in 96-well format. Well-to-read sequence is between 250 and 450 bases. DNA sequences collected through the ABI software are used to search the NCBI non-redundant nucleotide database using the NCBI BLAST program (Basic Local Alignment Search Tool, Altschul et al., 1997). Normally an expectation value threshold of 0.01 limits results to matches with homology of interest, and the top 100 descriptions and top 50 alignments from each BLAST result are saved to a file. The top 10 descriptions are added to a local database in order to ease searching for keywords in the descriptions.

### Example 2

Protein Sample Preparation

Extraction of protein from tissues: Fresh or frozen tissue of human origin for the purpose of this invention, is cut off in small pieces, grounded, homogenized in a Tris-HCl, pH7.5, 50 mM, EDTA 2 mM, NaCl 100 mM, NP40 1%, and vanadate 1 mM solution containing the following protease inhibitors: PMSF, aprotinin, leupeptin at 1, 2 and 4 mM respectively. The homogenate is kept on ice for 20 minutes and centrifuged at 14,000 rpm for 15 minutes. Supernatant is transferred to a new container and the tissue pellet is resuspended, and again kept on ice for 20 minutes and centrifuged as indicated above. Supernatant is removed and added to the first one. Protein concentration is determined according to standard conditions as known to those skilled in the art. Protein solution is stored in a −80 freezer until further usage.

Preparation of serum samples: Protein concentration of serum samples is determined using standard spectrophotometric methods as mentioned above. Protein concentration of serum samples greatly varies between 40 and 100 mg/ml. All samples are made 1.5 mg/ml by diluting in Tris-HCl buffer, pH 7.5.

### Example 3

Immunodetection of Gene Products by Matrix Protein Array

For the purpose of the invention, protein samples can be derived from human fresh or frozen tissues, mammalian cell cultures, patient sera or any other patient biological fluid, and prepared as described in Example 2. For the purpose of the invention, protein samples can also be derived from microdissected cell types from normal or disease tissue samples, or from a given subcellular compartment.

Matrix Protein Array: The solid support of the matrix protein array may be composed of a different number of compartments of different sizes, depending on the scope of the investigation. In a preferred embodiment of this invention, biological samples are spotted or printed (see below) in a matrix arrangement within each compartment on a nitrocellulose membrane. Each individual compartment is overlayed with an antibody (polyclonal, monoclonal, Fab fragment, monospecific, single chain, affibodies, or any other recombinant version of conventional or combinatorial antibodies).

Printing of total protein extracts: Two layers of precut blotting paper are placed in Omni-tray and are soaked with TNE solution (10 Tris-HCl, pH7.5; 2.5 mM EDTA, 50 mM NaCl). A precut sheet of nitrocellulose (Schleicher & Schuell) is placed over the blotting paper and wet by capillarity. To construct the matrix protein array, individual samples may be deposited either manually or with a robotic system (Genomic Solutions Flexys, PBA Robotics, UK). Routinely, 25 nl of a 1.5 mg/ml protein solution are spotted per sample, in duplicate when needed. The matrix protein arrays are dried on blotting papers for 1 h at RT.

Arrays are rehydrated by two rinses of 5 min each in TNE solution with constant rocking, incubated for 30 min at RT in 2% hydrogen peroxide in TNE (40 ml/tray), with constant rocking, to inactivate endogenous peroxidase, and thoroughly rinsed again in TNE (30 ml/tray). Protein arrays are then treated with TNET blocking solution (10 Tris-HCl, pH7.5, 2.5 mM EDTA, 50 mM NaCl, 0.1% Tween 20, containing 2.5% non fat dry milk) in a wide tray for 1 hour with constant rocking at room temperature. After blocking, the matrix protein arrays are given 2 quick rinses of 2 min each in TNE.

Antibodies: Antibodies are routinely used at 1:1,000-1:2,000 dilution, or more when needed. Dilution buffer is TNET containing 0.02% BSA, and 0.02% sodium azide. Aliquots of each antibody solution are added to each compartment, enough to cover, containing a matrix of protein arrays, as designed in a given experiment. Protein arrays are incubated with the antibodies 1 h at RT with constant shaking.

Detection of antigen-antibody complexes: Matrix protein arrays are washed five times in TNET and incubated with appropriate secondary antibodies conjugated with horseradish peroxydase (Roche Diagnostic Corp. Chicago, Ill.) diluted 1:10,000 in the blocking solution for 1 h at RT with constant rocking. Then matrix protein arrays are extensively washed 5 times, 10 minutes each, with constant rocking, in TNET without milk. Antigen-antibody complexes are routinely visualized using a peroxidase-based chemiluminescent reaction. After a water rinse, matrix protein arrays are incubated in the dark with 7.5 ml of a 1:1 luminol/enhancer substrate solution (Roche Molecular Diagnostic, Lumilight; SuperSignal™ CL-HRP Substrate System, Pierce, Ill.) and hydrogen peroxide for 5 min on the rocker. Matrix protein arrays are exposed to X-ray films (X-OMAT AR5, Kodak) from a few seconds to several minutes, until satisfactory detection of signal is obtained. Alternatively, signal is captured by a CCD camera and processed by a specialized software (see Example 5 below).

Protocols based on different labeling and detection systems, such as alkaline-phosphatase, biotin-streptavidine, or fluorophores can also be successfully performed within the scope of the present invention.

### Example 4

Expression Patterns of Gene Products in Normal and Cancer Tissues and Biological Fluids

Expression patterns, of relevant gene products can be assessed in different conditions, for example in protein extracts from normal and disease tissues, or protein samples from normal and disease biological fluids, such as serum, using the antibodies of the present invention. For the purpose of the present invention, the expression pattern of the cancer, specific gene products is determined in normal versus disease state by immunodetection using antibodies corresponding to the gene products under investigation and the matrix protein array, as described in Example 3. The matrix is arrayed with protein samples from the desired number of normal and disease specimens, either tissues or biological fluids from normal individuals and cancer patients, prepared as described in Example 2. Because the number of samples on the matrix protein array can vary, an almost unlimited number of combinations is possible. The expression pattern of the cancer specific gene products may also be assessed, as compared to normal, in different histologic types of diseased tissues, or at different cancer stages using tissues and biological fluids from patients at different stages of disease.

These experiments demonstrate the cancer specificity of each given gene product, and analysis of said experiments determine whether that gene product is underexpressed or overexpressed in relation to cancer.

### Example 5

Data Analysis

Quantification of antigen-antibody hybridization spots generated in Examples such as 3 and 4 is achieved through computer programs such as, but not limited to, the freely available Dapple program (Buhler et al., 2000, Improved techniques for finding spots on DNA microarrays, UW CSE Technical Report UWTR 2000-08-05; http://www.cs.wustl.edu/jbuhler/research/dapple/) that enables robust and high-throughput spot finding on computer image files, which may be generated by an image scanner (of, for example, an X-ray film), CCD (charge-coupled device), or digital camera, laser scanner, or any other means.

Dapple quantifies spots by determining the average intensity of all the pixels in the spot. The program calculates a background intensity as the mean intensity of the pixels of the rectangular grid for the spot after removal of those pixels that are in the circular spot. Such intensity quantifications generated in “normal versus disease” experiments or “normal versus disease stages”, or “normal versus histologic types of cancer”, or normal tissues distribution (see Example 4), enable those skilled in the art to attribute to cancer specific gene products of the present invention a value indicating the relative expression level for that gene product in the various groups of interest.

Antibodies with overall reactivity too low to be of practical interest are removed from further consideration. Further filters and thresholds may be applied by the biologist as appropriate. The spot intensities can then be normalized to remove any systematic experimental errors. Antibodies that show statistically significant discrimination between stages (or between any two groups of interest: i.e., normal and disease, various normal tissues, histological types of cancer) may then be selected via two-sample Welch t-statistics (p-value<0.05). The p-values can be adjusted with multiple testing procedures to control the family-wise Type I error rate or the false discovery rate. In the case of matched normal and diseased samples, a one-sample statistic is applied. The selected antibodies show some differential reactivity between lung cancer samples. Further statistical analysis, such as the nearest shrunken centroids method (Tibshirani et al., 1999, PNAS 10:6567-6572), then identifies a subset of the antibodies that best characterizes each disease stage or any group of interest. This classifier can be used further (Example 6) to predict the disease stage or type of an unknown sample from a measurement of the reactivity of the sample with the selected antibody subset. This method also allows the discovery of previously unknown classes, for example tumor types, from the data.

### Example 6

Diagnostic Analysis of Patients'Samples

To demonstrate the use of the polyclonal antibodies of the present invention in diagnostic applications, an unknown series of patients' samples, for example sera from patients affected with a variety of cancer types-and other diseases, is prepared for immunodetection analysis via the matrix protein array as described in Example 3.

In the case of a single-antibody diagnostic test, the reactivity of the antibody with each blind sample is measured and compared to the reactivity of the antibody with a control sample. Comparison of this data with known data for the antibody yields a probability that the blind sample belongs to a given type or stage of cancer. Based on these probabilities, a call is made for the blind sample. Analysis of the training set for the antibody gives the appropriate thresholds for a particular sensitivity and specificity of the diagnostic test. For example, analysis of the training set may indicate that classifying as diseased unknown samples that are >20% brighter than a control sample and classifying as normal unknown samples that are <10% brighter than the control sample produce a diagnostic test with 80% sensitivity and 80% specificity.

In the case of a multiple-antibody diagnostic test, a model is built from the reactivities of the multiple antibodies in the matrix protein array (Tibshirani et al., PNAS 1999, 10:6567-6572). The model specifies the relative importance of each antibody in the model. Antibodies whose reactivities are stable within stages or within classes have higher weight in the model. Such a model provides the probabilities that a sample belongs to each class. The probabilities are calculated based on the similarities of the reactivity patterns of the blind sample to reactivity patterns of known samples. Similar to the single-antibody case, analysis of the prior screening data yields the sensitivity and specificity of the diagnostic test when using various probability thresholds to make a call for the blind sample.

### Example 7

Prokaryotic and Eukaryotic Expression Systems

The expression of polynucleotide sequences of the present invention can be assayed in bacterial or mammalian systems and relevant gene products can be purified thereof. A strategy was designed to turn a plasmid containing a sequence of interest into a vector adequate for bacterial expression. The commercially available vector pDEST26, Ap^{R} (Invitrogen) was modified to harbor a “cassette” featuring: bacteriophage T7 promoter-lacO(operator)-bacterial ribosome binding site—His₆ tag (abbreviated T7H), followed by the selectable kanamycin resistance gene (Kn^{R}), and followed by another sequence containing: bacteriophage T7 promoter—LacI^{q} hybrid trp-lac promoter (abbreviated trcH). T7H and trcH enable expression of the sequence of interest in any orientation and optional purification as well. The cassette is flanked by XhoI and AscI sites, and is engineered in three different frames. The plasmid containing the sequence of interest, i.e. all or part of the relevant DNA sequences, is designated “target” plasmid for sake of clarity. The target plasmid must possess the unique XhoI or AscI site immediately upstream of the sequence of interest. Then, target plasmid and cassette containing vector are linearized by e.g. XhoI, the mixture is ligated and bacterial clones are selected for kanamaycin resistance. Only the target plasmid having incorporated the cassette will survive (the cassette containing plasmid harbors also the ccdB killer gene that is not suppressed in standard bacterial host strains, such as DH5 alfa).

Similarly, to turn any target plasmid into a mammalian expression vector, the pDONR201, Kn^{R} vector (Invitrogen) was modified to contain a cassette harboring the SV40 promoter, the ampicillin resistance gene (Ap^{R}) and the CMV promoter (cytomegalovirus immediate/early promoter). Two eukaryotic promoters are positioned in opposite orientation. The cassette is flanked by BamHI and EcoRI sites. In another version, the cassette is flanked by BamHI and XhoI sites. Similarly to the strategy outlined above for the bacterial expression vectors, target plasmids and mammalian cassette containing plasmids are cut by an appropriate restriction enzyme flanking the sequence of interest, ligated together, and the ligation products are transformed into bacterial cells followed by a selection for ampicillin resistance. The only survival is the cell carrying the target plasmid with incorporated cassette. Expression of the relevant cancer related gene product will be driven by either of the two mammalian promoters.

Molecular cloning techniques for introducing the relevant DNA sequences in the vectors described or in other vectors as mentioned are known to the skilled in the arts and have been extensively described (F. Ausubel et al., 1996, Current Protocols in Molecular Biology, John Wiley & Sons Ed; Sambrook, Fritsch and Maniatis, 1989, Molecular Cloning: A laboratory Manual, Cold Spring Harbor Laboratory Press).

### Example 8

Protein Expression in E.coli and Purification of Recombinant Proteins

Protein expression in bacterial cells and purification thereof can be performed by a variety of methods known to those skilled in the art, which greatly depend on the vectors and expression system selected (as described in F. Ausubel et al., 1996, Current Protocols in Molecular Biology, John Wiley & Sons Ed; J. Sambrook, Fritsch and Maniatis, 1989, Molecular Cloning: A laboratory Manual, Cold Spring Harbor Laboratory Press).

In a preferred embodiment of the present invention, all or part of the relevant polynucleotide sequences is cloned in the three reading frames into an expression vector, containing a T7 promoter and His6tag, as described in Example 10. The following protocol for protein expression and purification can be performed in a 96-well format or scaled up. IPTG (“isopropyl-B-D-thiogalacto-pyranoside”) inducible bacterial cells (such as bacterial strain BL26 (DE3) pLysE, Novagen, Madison, Wis.) containing the target plasmid expressing the gene product of interest are grown with appropriate antibiotics until an absorbance of 0.6 at 600 nm. IPTG is added to a final concentration of 1 mM and cells are grown for 3 h to induce protein expression.

Protein purification is achieved by nickel chelate affinity chromatography. Briefly, cells are lysed in 8 M Urea, 0.1 M NaH₂PO₄, 0.01 M Tris-HCl, pH 8.0, 300 mM NaCl, 3% NP-40, 40 units/ml benzonase nuclease, 2 mM PMSF. Cell lysate is then mixed with Ni-NTA His resin and incubated with constant shaking for 30 min at RT. Cell lysates are vacuum filtered, and resin bound protein is washed three times with 8 M Urea, 0.1 M NaH₂PO₄, 0.01 M Tris-HCl, pH7.0, 300 mM NaCl, 1% NP-40. Proteins are eluted from the resin upon incubation with elution buffer (8 M Urea, 50 mM sodium phosphate, pH 7.0, 150 mM imidazole). Efficiency of protein expression and purification is determined by spotting purified proteins on nitrocellulose membranes and probing them first with anti-His-tag monoclonal antibodies (Pierce, Ill.) and then with the gene product specific antibodies.

In the case of a mammalian expression system, transformed host cells such as CHO, HeLa or others, with the desired target vector containing the polynucleotide of interest and modified for mammalian expression as indicated in Example 10, are grown for several hours in the presence of radioactive S³⁵ or biotinylated-methionine. Cells are harvested by centrifugation then lysed in RIPA detergent buffer (150 mM NaCl. 1% NP-40, 0.1% SDS, 0.5% DOC, 50 mM Tris, pH 7.5). Antibody to the corresponding gene product is added to the protein solution to specifically capture the desired gene product. Immunoprecipitated gene products are analyzed by 12-15% SDS-PAGE, per standard methods (see Example 7).

### Example 9

Production of Polyclonal, Monoclonal and Monospecific Antibodies

Polyclonal antibodies against the cancer specific gene products of the present invention, can be generated in many conventional ways, by direct injection of the polypeptide expressed as described in Example 11, or by administering the polypeptide to an animal, preferably a non human mammal capable of immune response. Even a partial peptide sequence from the relevant polypeptide can be synthesized and injected or administered to an animal to raise antibodies capable of binding the whole native protein. Standard immunization methods producing polyclonal antibodies are known to those skilled in the art (Colligan et al., Current Protocoles in Immunology, Wiley Interscience).

Production of monoclonal and monospecific antibodies: Monoclonal antibodies are prepared using hybridoma technology (Kohler et al., Nature 256:495 (1975); Hammerling et al., in: Monoclonal Antibodies and T-Cell Hybridomas, Elsevier N.Y., (1981) pp. 563-681). Antibodies generated against the gene products corresponding to a sequence of the present invention can be obtained by direct injection of the gene product, its fragment or its derivatives into an animal, preferably a non-human. The antibody obtained will then bind the gene product itself. In this manner, even a sequence encoding only a fragment of the gene product can be used to generate antibodies binding to the whole native gene product. Such antibodies can then be used to isolate the gene product from tissue expressing that gene product.

When a polyclonal antibody with a reactivity of interest such as reactivity to a cancer specific protein is identified, monoclonal antibodies can be generated. This can be achieved by using either spleens of immunized animals (that generated the polyclonal serum of interest) or by immunizing de novo an animal, preferably a mouse, with that protein or fragment thereof. Four to five weeks after immunization, animals are bled to determine the presence of relevant antibodies in the serum. When sufficient antibodies displaying the relevant reactivity have been produced the splenocytes are extracted from the spleens of the immunized animals. Splenocytes are then fused with a suitable myeloma cell line, preferably the parent myeloma cell line (SP2/O; American Type Culture Collection, Rockville, Md.). After fusion, the resulting hybridoma cells are selectively maintained in appropriate selection medium (HAT), and then cloned by limiting dilution. After 10-14 days, the hybridoma medium supernatant obtained through such selection are then assayed to identify clones which secrete antibodies which dislpay the reactivity of interest, i.e. bind competitively with the polyclonal antibody sera in an assay measuring the binding activity of interest.

For preparation of monoclonal antibodies, any technique which provides antibodies produced by continuous cell lines cultures can be used. Examples include the hybridoma technique (Kohler and Milstein, 1975, Nature, 256:495-497), the trioma technique, the human B-cell hybridoma technique (Kozbor et al., 1983, Immunology Today 4:72) and the EBV-hybridoma technique to produce human monoclonal antibodies (Cole, et al., 1985, in Monoclonal Antibodies and Cancer Therapy, Alan R. Liss, Inc., pp. 77-96).

Over the years, the growing use of antibodies in all fields and the tremendous range of their application has led to the manipulation of the antibody genes, either to alter the constant (C) region with which the variable (V) regions are associated, or to introduce designed changes in the antibody combining site. Contemporary PCR methods make it feasible to clone the antibody V region genes encoding both the heavy and light chains of the hybridoma, to ligate these into plasmid expression vectors encoding constant regions of human or other species, and to express them after transfection in myeloma cell lines capable of high-level production.

Techniques described for the production of single chain antibodies (U.S. Pat. No. 4,946,778) can be adapted to produce single chain antibodies to immunogenic gene-product of this invention. Phage display techniques as described in McCafferty (“Phage display of peptides and proteins—A Laboratory Manual, Kay B. B., Winter J. & McCafferty J, Eds. Academic Press, 1996) and in U.S. Pat. No. (4,797,363 Teodorescu et al., U.S. Pat. No. 4,987,073 Berman et al., U.S. Pat. No. 5,223,409 Ladner et al, and U.S. Pat. No. 5,338,665 Schatz et al.) can also be used to produce antibodies against any gene product or fragment thereof and/or to mimic the interaction between an antibody and a gene product.

Production of monoclonal antibodies based on DNA immunization: In a preferred embodiment of the present invention, the preparation of polynucleotide template of different fragment of the same gene sequence may be used to perform genetic immunization with the aim of making monoclonal antibodies. Typically, a gene sequence coding for a gene product is divided into small fragments of 20, 30, 50, 60, 70, 80, 90 and 100 base pairs or more. The number of base pairs might be smaller, bigger or/and in between as well. These fragments may be of any length and may or may not overlap on between themselves. These polynucleotide sequence are then cloned in recombinant vector constructs which are used to transform bacteria, as it is known to those skilled in this art. Bacterial clones are grown individually and polynucleotide templates are prepared as described above for genetic immunization of mice. Five weeks after the first immunization, animals are bled and sera are used to test the presence of antibodies against the gene product encoded by the corresponding immunizing gene. When the immune reaction is judged satisfactory, the spleens of the animals are removed. Splenocytes are separated from the rest of cells and connective tissues and fused with myeloma cell lines such as Sp2/0. Hybrid clones are grown in an appropriate selection medium for 10 to 14 days and their medium supernatant is tested for the production of specific polyclonal antibodies against the corresponding gene product.

The Examples disclosed above are merely intended to illustrate the various utilities of this invention. It is understood that numerous modifications and variations of the present invention are possible in light of the above teachings and, therefore, within the scope of the appended claims, the present invention may be practiced otherwise than as particularly disclosed.

All patents and publications are herein incorporated for reference to the same extent as if each individual publication was specifically and individually indicated to be incorporated by reference. It should be understood that although the present invention has been specifically disclosed by preferred embodiments and optional features, modification and variation of the concepts herein disclosed may be resorted by those skilled in the art, and that such modifications and variations are considered to be within the scope of this invention.

TABLE 1A

| sampleID | source | type | stage | protein set |
|---|---|---|---|---|
| db-1 | tissue | bronchoalveolar ca | I-A | set 1 |
| db-2 | tissue | bronchoalveolar ca | I-A | set 1 |
| db-3 | tissue | bronchoalveolar ca | I-B | set 1 |
| db-4 | tissue | bronchoalveolar ca | 1 | set 1 |
| db-5 | tissue | bronchoalveolar ca | 1A | set 1 |
| db-6 | tissue | bronchoalveolar ca | 1B | set 1 |
| db-7 | tissue | bronchoalveolar ca | 1B | set 1 |
| db-8 | tissue | bronchoalveolar ca | 1 | set 1 |
| n-1 | tissue | normal | | set 1 |
| n-2 | tissue | normal | | set 1 |
| n-3 | tissue | normal | | set 1 |
| n-4 | tissue | normal | | set 1 |
| n-5 | tissue | normal | | set 1 |
| n-6 | tissue | normal | | set 1 |
| n-7 | tissue | normal | | set 1 |
| n-8 | tissue | normal | | set 1 |
| da-9 | tissue | adeno ca | I-B | set 2 |
| da-10 | tissue | adeno ca | I-B | set 2 |
| da-11 | tissue | adeno ca | I-B | set 2 |
| da-12 | tissue | adeno ca | I-B | set 2 |
| da-13 | tissue | adeno ca | I-B | set 2 |
| da-14 | tissue | adeno ca | 1 | set 2 |
| da-15 | tissue | adeno ca | 1A | set 2 |
| da-16 | tissue | adeno ca | 1B | set 2 |
| n-9 | tissue | normal | | set 2 |
| n-10 | tissue | normal | | set 2 |
| n-11 | tissue | normal | | set 2 |
| n-12 | tissue | normal | | set 2 |
| n-13 | tissue | normal | | set 2 |
| n-14 | tissue | normal | | set 2 |
| n-15 | tissue | normal | | set 2 |
| n-16 | tissue | normal | | set 2 |
| da-17 | tissue | adeno ca | 1A | set 3 |
| ds-18 | tissue | squamous cell ca | I-B | set 3 |
| ds-19 | tissue | squamous cell ca | I-B | set 3 |
| ds-20 | tissue | squamous cell ca | I-B | set 3 |
| ds-21 | tissue | squamous cell ca | I-B | set 3 |
| ds-22 | tissue | squamous cell ca | I-B | set 3 |
| ds-23 | tissue | squamous cell ca | I-A | set 3 |
| ds-24 | tissue | squamous cell ca | I-B | set 3 |
| n-17 | tissue | normal | | set 3 |
| n-18 | tissue | normal | | set 3 |
| n-19 | tissue | normal | | set 3 |
| n-20 | tissue | normal | | set 3 |
| n-21 | tissue | normal | | set 3 |
| n-22 | tissue | normal | | set 3 |
| n-23 | tissue | normal | | set 3 |
| n-24 | tissue | normal | | set 3 |
| ds-25 | tissue | squamous cell ca | I-B | set 4 |
| ds-26 | tissue | squamous cell ca | I-B | set 4 |
| ds-27 | tissue | squamous cell ca | I-B | set 4 |
| ds-28 | tissue | squamous cell ca | I | set 4 |
| ds-29 | tissue | squamous cell ca | I | set 4 |
| ds-30 | tissue | squamous cell ca | I | set 4 |
| ds-31 | tissue | squamous cell ca | 1 | set 4 |
| ds-32 | tissue | squamous cell ca | 1 | set 4 |
| n-25 | tissue | normal | | set 4 |
| n-26 | tissue | normal | | set 4 |
| n-27 | tissue | normal | | set 4 |
| n-28 | tissue | normal | | set 4 |
| n-29 | tissue | normal | | set 4 |
| n-30 | tissue | normal | | set 4 |
| n-31 | tissue | normal | | set 4 |
| n-32 | tissue | normal | | set 4 |
| n-33 | tissue | normal | | set 5 |
| n-34 | tissue | normal | | set 5 |
| n-35 | tissue | normal | | set 5 |
| n-36 | tissue | normal | | set 5 |
| da-33 | tissue | adeno ca | 1 | set 5 |
| da-34 | tissue | adeno ca | 1 | set 5 |
| da-35 | tissue | adeno ca | 1 | set 5 |
| da-36 | tissue | adeno ca | 1 | set 5 |
| n-37 | plasma | normal | | set 6 |
| n-38 | plasma | normal | | set 6 |
| n-39 | plasma | normal | | set 6 |
| n-40 | plasma | normal | | set 6 |
| n-41 | plasma | normal | | set 6 |
| n-42 | plasma | normal | | set 6 |
| n-43 | plasma | normal | | set 6 |
| n-44 | plasma | normal | | set 6 |
| da-45 | plasma | adeno ca | 1 | set 6 |
| da-46 | plasma | adeno ca | 1 | set 6 |
| da-47 | plasma | adeno ca | 1 | set 6 |
| da-48 | plasma | adeno ca | 1 | set 6 |
| da-49 | plasma | adeno ca | 1 | set 6 |
| da-50 | plasma | adeno ca | 1 | set 6 |
| da-51 | plasma | adeno ca | 1 | set 6 |
| da-52 | plasma | adeno ca | 1 | set 6 |

TABLE 1B
set 1: Bronchoalveolar carcinoma/tissue

| | | | |
|---|---|---|---|
| db-1 | db-2 | db-3 | db-4 |
| db-5 | db-6 | db-7 | db-8 |
| n-1 | n-2 | n-3 | n-4 |
| n-5 | n-6 | n-7 | n-8 |

set 2: Adenocarcinoma/tissue

| | | | |
|---|---|---|---|
| da-9 | da-10 | da-11 | da-12 |
| da-13 | da-14 | da-15 | da-16 |
| n-9 | n-10 | n-11 | n-12 |
| n-13 | n-14 | n-15 | n-16 |

set 3: Squamous cell carcinoma/tissue

| | | | |
|---|---|---|---|
| da-17 | ds-18 | ds-19 | ds-20 |
| ds-21 | ds-22 | ds-23 | ds-24 |
| n-17 | n-18 | n-19 | n-20 |
| n-21 | n-22 | n-23 | n-24 |

set 4: Squamous cell carcinoma/tissue

| | | | |
|---|---|---|---|
| ds-25 | ds-26 | ds-27 | ds-28 |
| ds-29 | ds-30 | ds-31 | ds-32 |
| n-25 | n-26 | n-27 | n-28 |
| n-29 | n-30 | n-31 | n-32 |

set 5: Adenocarcinoma/tissue

| | | | |
|---|---|---|---|
| n-33 | n-33 | da-33 | da-33 |
| n-34 | n-34 | da-34 | da-34 |
| n-35 | n-35 | da-35 | da-35 |
| n-36 | n-36 | da-36 | da-36 |

set 6: Adenocarcinoma/plasma

| | | | |
|---|---|---|---|
| n-37 | n-38 | n-39 | n-40 |
| n-41 | n-42 | n-43 | n-44 |
| da-45 | da-46 | da-47 | da-48 |
| da-49 | da-50 | da-51 | da-52 |

TABLE 2

| | Prev- | | | | | |
|---|---|---|---|---|---|---|
| Ab ID | alence | D/N | N | reactive samples | regulation | protein set |
| 5013 | 0.500 | 0.605 | 8 | da-9 da-11 da-12 | down | set 2 |
| | | | | da-10 | | |
| 5016 | 0.000 | 1.210 | 4 | | up | set 5 |
| 5030 | 0.375 | 0.605 | 8 | da-10 da-11 da-9 | down | set 2 |
| 5050 | 0.000 | 0.749 | 8 | | down | set 2 |
| 5050 | 0.000 | 0.844 | 7 | | down | set 3 + 4 |
| 5193 | 0.000 | 0.850 | 8 | | down | set 2 |
| 5248 | 0.250 | 1.352 | 4 | da-35 | up | set 5 |
| 5269 | 0.000 | 1.258 | 4 | | up | set 5 |
| 5320 | 0.000 | 0.784 | 8 | | down | set 2 |
| 5350 | 0.000 | 0.850 | 8 | | down | set 2 |
| 5588 | 0.500 | 1.472 | 4 | da-36 da-35 | up | set 5 |
| 5695 | 0.500 | 1.587 | 4 | da-33 da-36 | up | set 5 |
| 5872 | 0.250 | 1.259 | 4 | da-35 | up | set 5 |
| 5911 | 0.000 | 0.852 | 8 | | down | set 2 |
| 5911 | 0.000 | 0.866 | 7 | | down | set 3 + 4 |
| 5984 | 0.000 | 0.798 | 7 | | down | set 3 + 4 |
| 6050 | 0.250 | 1.361 | 4 | da-33 | up | set 5 |
| 6068 | 0.375 | 0.628 | 8 | da-11 da-15 da-14 | down | set 2 |
| 6068 | 0.000 | 0.872 | 4 | | down | set 5 |
| 6087 | 0.500 | 1.468 | 4 | da-35 da-33 | up | set 5 |
| 6112 | 0.250 | 1.409 | 4 | da-35 | up | set 5 |
| 6202 | 0.000 | 1.287 | 4 | | up | set 5 |
| 6329 | 0.500 | 1.836 | 4 | da-36 da-35 | up | set 5 |
| 6361 | 0.250 | 1.353 | 4 | da-35 | up | set 5 |
| 6395 | 0.000 | 1.228 | 4 | | up | set 5 |
| 6401 | 0.750 | 1.782 | 4 | da-33 da-35 da-36 | up | set 5 |
| 6789 | 0.375 | 0.556 | 8 | ds-30 ds-27 ds-26 | down | set 3 + 4 |
| 6970 | 0.250 | 1.414 | 4 | da-35 | up | set 5 |
| 7125 | 0.500 | 1.389 | 4 | da-35 da-36 | up | set 5 |
| 5854-A | 0.000 | 1.271 | 4 | | up | set 5 |
| D630 | 0.375 | 1.408 | 8 | da-16 da-12 da-11 | up | set 2 |
| D686 | 0.125 | 0.743 | 8 | ds-30 | down | set 3 + 4 |
| D687 | 0.000 | 0.740 | 8 | | down | set 2 |
| H1015 | 0.375 | 1.362 | 8 | da-15 da-9 da-11 | up | set 2 |
| H1211 | 0.250 | 1.330 | 8 | da-15 da-9 | up | set 2 |
| H1244 | 0.125 | 1.305 | 8 | da-15 | up | set 2 |
| J2082 | 0.375 | 1.395 | 8 | da-15 da-14 da-11 | up | set 2 |
| J2082 | 0.750 | 1.675 | 4 | da-35 da-36 da-33 | up | set 5 |
| J2094 | 0.375 | 1.502 | 8 | da-15 da-11 da-14 | up | set 2 |
| J2094 | 0.375 | 1.433 | 8 | ds-25 ds-27 ds-30 | up | set 3 + 4 |
| J2151 | 0.250 | 0.573 | 8 | ds-25 ds-28 | down | set 3 + 4 |
| J2232 | 0.000 | 1.177 | 8 | | up | set 2 |
| J2257 | 0.000 | 1.281 | 8 | | up | set 2 |
| J2554 | 0.143 | 0.662 | 7 | ds-20 | down | set 3 + 4 |
| J2555 | 0.000 | 0.698 | 7 | | down | set 3 + 4 |
| J2727 | 0.000 | 0.820 | 4 | | down | set 5 |
| J2941 | 0.375 | 1.470 | 8 | db-2 db-3 db-1 | up | set 1 |
| J2992 | 0.500 | 1.484 | 8 | da-16 da-10 | up | set 2 |
| | | | | da-14 da-13 | | |
| L0925 | 1.000 | 0.347 | 8 | da-14 da-9 da-13 | down | set 2 |
| | | | | da-10 da-15 da-11 | | |
| | | | | da-16 da-12 | | |
| L0925 | 0.000 | 0.699 | 7 | | down | set 3 + 4 |
| L0925 | 0.000 | 0.671 | 4 | | down | set 5 |
| L0937 | 0.571 | 1.568 | 7 | ds-18 ds-19 | up | set 3 + 4 |
| | | | | ds-23 ds-24 | | |
| L2027 | 0.500 | 1.680 | 4 | da-36 da-35 | up | set 5 |
| L2331 | 0.000 | 0.866 | 8 | | down | set 1 |
| L2391 | 0.000 | 0.848 | 4 | | down | set 5 |
| M0448 | 0.625 | 0.575 | 8 | da-13 da-16 da-14 | down | set 2 |
| | | | | da-10 da-15 | | |
| M0448 | 0.286 | 0.573 | 7 | ds-18 ds-21 | down | set 3 + 4 |
| M0448 | 0.250 | 0.764 | 4 | da-34 | down | set 5 |
| M2148 | 0.000 | 1.111 | 8 | | up | set 2 |
| M2322 | 0.500 | 1.387 | 8 | da-13 da-9 | up | set 2 |
| | | | | da-15 da-14 | | |
| M2953 | 0.125 | 0.758 | 8 | da-11 | down | set 2 |
| M2953 | 0.000 | 0.949 | 7 | | down | set 3 + 4 |
| M2957 | 0.125 | 0.754 | 8 | da-11 | down | set 2 |
| O1093 | 0.125 | 0.802 | 8 | da-11 | down | set 2 |
| O2096 | 0.000 | 0.904 | 8 | | down | set 3 + 4 |
| O2494 | 0.250 | 1.285 | 4 | da-35 | up | set 5 |
| P0246 | 0.000 | 1.157 | 8 | | up | set 2 |
| P0330 | 0.000 | 1.177 | 8 | | up | set 2 |
| P1395 | 0.000 | 1.224 | 8 | | up | set 3 + 4 |
| P1398 | 0.125 | 1.240 | 8 | da-15 | up | set 2 |
| P1398 | 0.500 | 1.411 | 8 | ds-30 ds-25 ds-31 | up | set 3 + 4 |
| | | | | ds-32 | | |
| P1398 | 0.500 | 1.439 | 4 | da-36 da-35 | up | set 5 |
| P1514 | 0.500 | 1.889 | 4 | da-36 da-35 | up | set 5 |
| P2581 | 0.000 | 0.851 | 8 | | down | set 3 + 4 |
| P2582 | 0.000 | 0.829 | 8 | | down | set 1 |
| P2582 | 0.000 | 0.885 | 8 | | down | set 3 + 4 |
| P2631 | 0.000 | 1.258 | 8 | | up | set 2 |
| P2635 | 0.000 | 1.084 | 8 | | up | set 2 |
| P2658 | 0.000 | 1.216 | 8 | | up | set 3 + 4 |
| P2944 | 0.250 | 1.301 | 8 | ds-30 ds-25 | up | set 3 + 4 |
| Q0002 | 0.125 | 1.213 | 8 | db-7 | up | set 1 |
| Q0062 | 0.375 | 1.317 | 8 | db-7 db-4 db-6 | up | set 1 |
| Q0892 | 0.375 | 1.329 | 8 | ds-25 ds-30 ds-32 | up | set 3 + 4 |

TABLE 3

| Ab ID | Prevalence | D/N | N | reactive samples | regulation | protein set |
|---|---|---|---|---|---|---|
| 5055 | 0.571 | 1.56 | 7 | da-52 da-46 da-48 da-49 | up | set 6 |
| 5070 | 0.429 | 1.41 | 7 | da-52 da-48 da-46 | up | set 6 |
| 5193 | 0.571 | 1.41 | 7 | da-48 da-52 da-46 da-49 | up | set 6 |
| 5248 | 0.571 | 1.35 | 7 | da-48 da-46 da-49 da-52 | up | set 6 |
| 5332 | 0.429 | 1.27 | 7 | da-46 da-48 da-49 | up | set 6 |
| 5445 | 1.000 | 2.90 | 7 | da-48 da-52 da-46 da-49 da-50 da-47 da-51 | up | set 6 |
| 5544 | 1.000 | 1.99 | 7 | da-48 da-49 da-46 da-52 da-50 da-51 da-47 | up | set 6 |
| 5751 | 1.000 | 2.66 | 7 | da-48 da-46 da-52 da-49 da-47 da-50 da-51 | up | set 6 |
| 5944 | 0.571 | 1.35 | 7 | da-49 da-48 da-46 da-52 | up | set 6 |
| 5956 | 0.286 | 1.26 | 7 | da-48 da-46 | up | set 6 |
| 6032 | 0.571 | 1.93 | 7 | da-48 da-52 da-46 da-49 | up | set 6 |
| 6067 | 0.429 | 1.43 | 7 | da-52 da-48 da-46 | up | set 6 |
| 6068 | 1.000 | 6.79 | 7 | da-46 da-48 da-52 da-49 da-47 da-50 da-51 | up | set 6 |
| 6104 | 0.571 | 1.81 | 7 | da-48 da-46 da-52 da-49 | up | set 6 |
| 6137 | 0.429 | 0.63 | 7 | da-52 da-51 da-50 | do | set 6 |
| 6155 | 0.429 | 1.56 | 7 | da-48 da-46 da-52 | up | set 6 |
| 6234 | 0.571 | 1.59 | 7 | da-48 da-46 da-52 da-49 | up | set 6 |
| 6301 | 0.714 | 2.03 | 7 | da-48 da-46 da-52 da-49 da-50 | up | set 6 |
| 6329 | 0.571 | 1.57 | 7 | da-48 da-46 da-52 da-49 | up | set 6 |
| 6365 | 0.857 | 2.13 | 7 | da-48 da-52 da-46 da-49 da-50 da-47 | up | set 6 |
| 6501 | 0.571 | 1.40 | 7 | da-46 da-49 da-48 da-52 | up | set 6 |
| 6567 | 0.857 | 1.73 | 7 | da-49 da-50 da-46 da-51 da-52 da-48 | up | set 6 |
| 6680 | 0.429 | 1.29 | 7 | da-46 da-48 da-49 | up | set 6 |
| 6887 | 0.571 | 1.68 | 7 | da-48 da-52 da-46 da-49 | up | set 6 |
| 6891 | 0.429 | 1.38 | 7 | da-49 da-52 da-46 | up | set 6 |
| 6904 | 0.429 | 1.42 | 7 | da-48 da-46 da-49 | up | set 6 |
| 7115 | 0.857 | 0.38 | 7 | da-49 da-51 da-50 da-47 da-48 da-52 | do | set 6 |
| 7139 | 0.000 | 0.65 | 7 | | do | set 6 |
| 7393 | 0.857 | 2.74 | 7 | da-46 da-48 da-49 da-52 da-50 da-51 | up | set 6 |
| A1084 | 0.375 | 1.58 | 8 | da-46 da-48 da-49 | up | set 6 |
| A1245 | 0.500 | 1.51 | 8 | da-46 da-48 da-49 da-52 | up | set 6 |
| A1325 | 1.000 | 2.37 | 8 | da-46 da-48 da-49 da-52 da-50 da-51 da-47 da-45 | up | set 6 |
| A1811 | 0.625 | 1.48 | 8 | da-48 da-46 da-45 da-52 da-49 | up | set 6 |
| A1890 | 0.375 | 1.45 | 8 | da-46 da-48 da-52 | up | set 6 |
| A1911 | 0.500 | 1.66 | 8 | da-46 da-48 da-49 da-52 | up | set 6 |
| A2374 | 0.625 | 1.65 | 8 | da-46 da-48 da-49 da-52 da-50 | up | set 6 |
| A377 | 0.625 | 1.81 | 8 | da-46 da-48 da-49 da-52 da-47 | up | set 6 |
| AB578 | 0.625 | 1.64 | 8 | da-46 da-48 da-52 da-49 da-45 | up | set 6 |
| AB597 | 0.875 | 4.87 | 8 | da-48 da-46 da-52 da-49 da-47 da-50 da-51 | up | set 6 |
| AB750 | 0.750 | 1.61 | 8 | da-46 da-47 da-48 da-49 da-52 da-50 | up | set 6 |
| AC151 | 0.375 | 1.40 | 8 | da-48 da-52 da-46 | up | set 6 |
| B36 | 0.875 | 2.47 | 8 | da-46 da-48 da-49 da-52 da-47 da-50 da-51 | up | set 6 |
| B49 | 0.125 | 0.65 | 8 | da-50 | do | set 6 |
| C1439 | 0.500 | 1.74 | 8 | da-48 da-46 da-45 da-52 | up | set 6 |
| C1493 | 0.625 | 1.71 | 8 | da-46 da-48 da-45 da-49 da-52 | up | set 6 |
| C1535 | 0.625 | 2.21 | 8 | da-48 da-52 da-46 da-49 da-47 | up | set 6 |
| C1735 | 0.375 | 1.51 | 8 | da-45 da-46 da-49 | up | set 6 |
| C1791 | 0.625 | 1.72 | 8 | da-46 da-48 da-49 da-45 da-52 | up | set 6 |
| C1875 | 0.500 | 1.33 | 8 | da-46 da-48 da-52 da-45 | up | set 6 |
| C1920 | 0.500 | 1.51 | 8 | da-48 da-46 da-52 da-49 | up | set 6 |
| C2099 | 0.625 | 2.64 | 8 | da-48 da-46 da-52 da-49 da-47 | up | set 6 |
| C2221 | 0.500 | 1.69 | 8 | da-48 da-52 da-46 da-49 | up | set 6 |
| C2277 | 0.750 | 2.10 | 8 | da-46 da-48 da-52 da-49 da-47 da-50 | up | set 6 |
| C2366 | 0.750 | 1.68 | 8 | da-46 da-52 da-48 da-49 da-47 da-45 | up | set 6 |
| C2836 | 0.375 | 1.35 | 8 | da-45 da-46 da-52 | up | set 6 |
| C2861 | 0.375 | 1.44 | 8 | da-45 da-46 da-49 | up | set 6 |
| C2911 | 0.625 | 2.61 | 8 | da-48 da-46 da-49 da-52 da-47 | up | set 6 |
| C2929 | 0.500 | 1.51 | 8 | da-45 da-46 da-51 da-47 | up | set 6 |
| C2952 | 0.625 | 1.70 | 8 | da-46 da-45 da-48 da-49 da-52 | up | set 6 |
| C2957 | 0.375 | 1.37 | 8 | da-46 da-48 da-52 | up | set 6 |
| D236 | 0.500 | 1.46 | 8 | da-46 da-47 da-48 da-49 | up | set 6 |
| D26 | 0.375 | 1.70 | 8 | da-45 da-46 da-52 | up | set 6 |
| D35 | 0.375 | 1.51 | 8 | da-46 da-45 da-49 | up | set 6 |
| J2105 | 0.375 | 1.81 | 8 | da-48 da-46 da-52 | up | set 6 |
| J2240 | 0.500 | 1.79 | 8 | da-48 da-46 da-52 da-49 | up | set 6 |
| L1975 | 0.625 | 1.76 | 8 | da-52 da-48 da-46 da-49 da-47 | up | set 6 |
| L2331 | 0.500 | 1.76 | 8 | da-46 da-48 da-49 da-52 | up | set 6 |
| L2892 | 0.500 | 1.48 | 8 | da-46 da-48 da-52 da-49 | up | set 6 |
| M2433 | 0.500 | 1.59 | 8 | da-48 da-46 da-52 da-49 | up | set 6 |
| M2829 | 0.500 | 1.70 | 8 | da-46 da-52 da-48 da-49 | up | set 6 |
| O0860 | 0.500 | 2.50 | 8 | da-48 da-46 da-49 da-52 | up | set 6 |
| O1036 | 0.500 | 1.74 | 8 | da-46 da-48 da-52 da-49 | up | set 6 |
| O1039 | 0.625 | 3.11 | 8 | da-48 da-46 da-52 da-49 da-47 | up | set 6 |
| O1117 | 0.250 | 1.39 | 8 | da-46 da-47 | up | set 6 |
| O1134 | 0.875 | 6.45 | 8 | da-48 da-52 da-46 da-49 da-47 da-50 da-51 | up | set 6 |
| O1200 | 0.250 | 1.46 | 8 | da-46 da-48 | up | set 6 |
| O1336 | 0.875 | 2.43 | 8 | da-48 da-46 da-52 da-49 da-47 da-50 da-51 | up | set 6 |
| O2602 | 0.500 | 0.61 | 8 | da-52 da-50 da-49 da-51 | do | set 6 |
| O2904 | 0.375 | 1.37 | 8 | da-45 da-48 da-46 | up | set 6 |
| P0764 | 0.500 | 1.40 | 8 | da-48 da-46 da-49 da-52 | up | set 6 |
| P1322 | 0.500 | 1.72 | 8 | da-48 da-46 da-49 da-52 | up | set 6 |
| P1398 | 0.375 | 1.42 | 8 | da-48 da-46 da-45 | up | set 6 |
| P1514 | 0.375 | 1.27 | 8 | da-52 da-46 da-48 | up | set 6 |
| P1684 | 0.375 | 1.39 | 8 | da-52 da-46 da-48 | up | set 6 |
| P1886 | 0.375 | 1.38 | 8 | da-46 da-52 da-48 | up | set 6 |
| P2225 | 0.375 | 0.55 | 8 | da-49 da-51 da-50 | do | set 6 |
| P2229 | 0.375 | 0.54 | 8 | da-49 da-51 da-50 | do | set 6 |
| P2315 | 0.625 | 1.57 | 8 | da-48 da-49 da-46 da-45 da-52 | up | set 6 |
| Q0067 | 0.500 | 1.79 | 8 | da-48 da-46 da-49 da-52 | up | set 6 |
| Q0504 | 0.375 | 1.33 | 8 | da-48 da-46 da-49 | up | set 6 |
| Q0635 | 0.125 | 1.22 | 8 | da-46 | up | set 6 |
| Q0728 | 0.250 | 1.29 | 8 | da-48 da-46 | up | set 6 |
| Q0959 | 0.500 | 0.50 | 8 | da-49 da-48 da-50 da-51 | do | set 6 |
| Q1021 | 0.375 | 0.54 | 8 | da-48 da-51 da-50 | do | set 6 |
| Q1142 | 0.375 | 1.40 | 8 | da-46 da-48 da-52 | up | set 6 |
| Q1254 | 0.375 | 0.60 | 8 | da-50 da-49 da-51 | do | set 6 |

| Antibody | |
|---|---|
| ID No | Polynucleotide Sequence Accession Numbers |
| 5050 | AF267861 AK055058 AK058051 AK058082 AY043301 BC008587 BC009875 |
| | BC010735 BC012891 BC014224 |
| 5055 | AC010273 AC104380 AF248053 AF321240 AK055548 AL031055 AL137188 |
| | AL158093 AP002979 NM_030777 |
| 5070 | AC090644 D86965 NM_014744 |
| 5193 | AB019219 AF026031 AF221842 AK001554 AK081998 AL137320 BC001666 |
| | BC005801 NM_012469 NM_133701 |
| 5248 | AB063285 AP000698 AP001726 |
| 5269 | AC004890 AC005550 AC018868 AC116533 AK096711 AP001086 BC001781 |
| | BC031015 NM_021029 XM_208185 XM_208369 XM_209835 XM_300270 |
| 5320 | AB022023 AF139055 AK026977 AK029236 AK122578 BC000280 M69181 |
| | NM_174834 XM_109944 XM_290747 |
| 5350 | AF346975 AF346981 AF347002 AF347004 AF347005 AF347006 AF347007 |
| | AF347011 AF381995 AY195764 |
| 5445 | AC007227 AK056143 AL135927 BC014507 BC033088 NM_005572 NM_170707 |
| | NM_170708 X03444 X03445 |
| 5751 | AK016720 AK037037 AY317140 BC006803 BC031828 BC047544 D12644 |
| | NM_004520 NM_008442 Y08319 |
| 5854-A | AC108863 AF270686 AL592166 NR_001457 X67247 X97583 |
| 5872 | AK075504 AK094047 AL353662 AP001922 AP002815 BC014623 BC036298 |
| | D83174 NM_001235 X61598 |
| 5911 | AC006028 AK024834 AK025582 AK025894 Z65202 Z65203 |
| 5944 | AC007546 AF139540 AF250326 AF332010 AK013424 AK091549 BC004536 |
| | BC005396 NM_014055 NM_031473 |
| 5984 | AD000712 NM_000444 U60475 U75645 U81174 U82970 U87284 Y08118 Y08119 |
| | Y10196 |
| 6032 | AC005995 AC073343 |
| 6050 | AB093286 AF116827 AK004100 AK026638 AL512505 BC025427 BC027469 |
| | BC051723 NM_026225 XM_053233 |
| 6087 | AC105141 AC105206 AK058094 AY337315 BC005913 J03517 J03553 J03890 |
| | NM_003018 U02948 |
| 6137 | AK022869 AK081329 AL732366 AL808146 X68365 XM_288144 |
| 6202 | AB044750 AC025871 AC084261 AF213023 AF263928 AK002050 AK023418 |
| | AK098243 BC001237 NM_018660 |
| 6301 | AC007541 AF323921 AK001731 AK005698 AK021783 AK022770 AK024021 |
| | BC020522 NM_152261 XM_234992 |
| 6329 | AB023229 AC022960 AK001662 AK017149 AL136749 BC043669 BC048382 |
| | NM_014939 XM_128964 XM_214614 |
| 6361 | AB018337 AC069257 AC087556 AK026451 BC028986 BC047420 XM_087353 |
| | XM_221375 |
| 6365 | AC003688 AC120057 AK056083 AK056956 AK097243 BC000399 BC012912 |
| | D43682 L46590 NM_000018 |
| 6401 | AC067945 AF097492 AF097494 AF158555 AF490841 AK025021 |
| 6501 | AB055890 AC087286 AF387101 AF406992 AK026186 BC000269 BC009213 |
| | M90360 NM_006738 NM_007200 |
| 6567 | AC008641 AC008666 AY310878 |
| 6887 | AB096243 AL109923 AP000924 BC010284 BC017063 J03548 M18003 M23668 |
| | M34786 M34787 M34788 NG_001117 NM_004109 |
| 6891 | AF346981 AF346982 AF346983 AF346988 AF347001 AF347002 AF347005 |
| | AF347006 AF347007 AF347011 AY195776 AY195778 AY195779 AY195781 |
| | AY195782 AY195784 AY195785 AY195786 AY195788 AY195792 |
| 6904 | AB018399 AC016912 AF045160 BC051338 D89787 NM_001430 NM_174725 |
| | U51626 U81983 U81984 |
| 7115 | AB028998 AF417490 AF518728 AF518729 AL137564 BC025818 BC042190 |
| | BC054099 NM_015319 NM_170754 |
| 7139 | AC006020 AC008493 AC010096 AC022467 AC112211 AL161729 AL161751 |
| | AL355797 AL355835 AL390058 AL450109 |
| 7393 | AC012454 AF400074 BC032785 J02761 M16764 M19097 M24461 NM_000542 |
| | NM_147779 XM_192996 |
| A1084 | AB074899 AL139194 AY164662 AY164728 L10333 L10334 L49144 NM_021136 |
| | NM_053865 U17604 |
| A1811 | AK007550 AK008204 AK031551 BC005338 BT006735 BT007970 NM_006136 |
| | U03269 U03851 U16741 |
| A1890 | AF135593 AF327407 AK051658 AK074325 BC044851 BC049916 NM_014396 |
| | NM_080631 NM_172120 U87309 |
| A1911 | AF347000 AF347003 AF347006 AF347007 AF347010 AF347011 AF347012 |
| | AF347013 AF347014 AF347015 |
| A2374 | AB058751 AF257319 AF258589 AF426314 AL592211 AL832592 BC005843 |
| | BC014635 NM_020145 NM_139302 |
| A377 | AF403770 AK092286 BC011353 BT006726 BT008284 M15856 NM_000237 U18091 |
| | X14390 X54516 |
| AB578 | BC009983 |
| AB750 | BC011940 |
| AC151 | BC014037 |
| B36 | AC007215 AF078843 AF190165 AJ276704 AK010310 AK054574 AK076982 |
| | AL136666 AL833469 BC009379 NM_016355 |
| C1493 | AF347001 AF347002 AF347004 AF347005 AF347006 AF347010 AF347012 |
| | AF347013 AF347014 AF347015 |
| C1535 | AF347003 AF347004 AF347005 AF347006 AF347007 AF347011 AF347012 |
| | AF347013 AF347014 AF347015 |
| C1735 | AC040958 AC127029 AF239249 AF239250 AF239251 AF239252 J03071 K00470 |
| | M13438 NG_001334 |
| C1791 | AC019133 AC090312 AJ318054 AL117648 BC038292 NM_003137 U09564 Z99128 |
| C2099 | AC093384 AC105300 AF033665 AK033327 BC004085 BC034113 D28476 |
| | NM_004238 NM_133975 |
| C2277 | AC005062 AC073587 AC087190 AC093591 BC001603 BC007505 BC019002 |
| | NM_000982 U14967 X89401 |
| C2836 | AC037476 AC092118 AF237583 AF300437 AF300449 AL122127 AP001028 J00228 |
| | NG_001019 Z17370 |
| C2861 | AF134157 AL035665 BC036689 NM_005461 |
| C2911 | AC004098 AC015917 AL133380 AL139378 AL513284 BC002866 M77233 |
| | NM_001011 XM_012638 XM_015717 |
| C2929 | AC087525 AK000158 AK009873 AL050025 BC031385 BC033432 BC035941 |
| | NM_017689 NM_178396 XM_134913 |
| C2952 | AC009336 AC015584 AC116665 AF154915 AK054090 AL928644 NM_021192 |
| | X60395 X60762 X71422 |
| D630 | AB057597 AJ132545 AK027573 AK031814 AL359540 AL683847 BC029766 |
| | BC033085 NM_007170 NM_146151 |
| D686 | AF220542 AF485818 AK074734 AK075510 AK075532 BC008734 BT006828 |
| | BT007519 NM_004107 U12255 |
| H1015 | AC004062 AF052112 AF077198 AF081281 AF291053 AL365267 BC008652 |
| | BC010397 NG_001063 NM_006330 |
| H1211 | AF119840 AF542069 BC034023 BC034026 BC035969 BC036003 BC039235 |
| | NM_000477 U22961 V00494 |
| H1244 | AB020866 AC087273 AC087625 AP006303 |
| J2082 | AF300628 AF412572 AK096858 BC008406 L06850 M24795 M98398 M98399 |
| | NM_000072 S67532 |
| J2094 | AC097653 |
| J2105 | AC004499 AC008686 AC073427 AC092849 AC097064 AC114791 AL031255 |
| | AP002770 AP003531 Z73420 |
| J2151 | AF237981 AF237982 AK031733 AL035670 BC010358 BC021418 BC037663 |
| | NM_016593 NM_018887 XM_223745 |
| J2240 | AC002420 AL805976 AL845473 BC035972 L47176 M22400 NM_004484 |
| | NM_012774 U50410 Z37987 |
| J2257 | AF347001 AF347002 AF347003 AF347005 AF347006 AF347007 AF347010 |
| | AF347011 AF347012 AF347013 |
| J2554 | AK074742 AY255168 AY255171 AY255172 AY255173 AY255175 AY255177 |
| | AY255178 AY255180 AY289079 |
| J2555 | AC016596 AC034102 AF026844 AK026741 BC014383 BC015984 BC032611 |
| | BC041318 NM_021104 Z12962 |
| J2727 | AB029499 AF112227 AF153979 AK031101 BC006088 BC022901 NM_006811 |
| | NM_012032 U49188 Z97053 |
| J2941 | AK001090 AK090498 AL683870 BC002508 BC010089 NM_004192 Y15521 |
| J2992 | AP005329 AY320408 BC016372 BC031972 BC032748 D50372 D82059 NM_006471 |
| | U26162 X54304 |
| L0925 | AF035316 AL031963 AL050056 AL445309 BC001194 BC001352 BC018780 |
| | NM_001069 NM_178012 X79535 |
| L0937 | AC004883 AC005080 AC005098 AC006995 AC083884 AC105418 AC115105 |
| | AC134616 AC134866 AC138783 |
| L1975 | AC004869 AC010726 AC091563 AC106767 AC106792 AL354723 AL603832 |
| | AP002962 AP005624 U73649 |
| L2027 | AC091103 AC091111 AC113267 |
| L2331 | AB011116 AC023830 AK011747 AK088533 BC014199 BC046830 BC050389 |
| | NM_029657 XM_048119 XM_220160 |
| L2892 | AC099314 AC126773 AK031265 AK045041 BC014462 BC041846 BC052189 |
| | NM_001793 X06340 X63629 |
| M0448 | AK092877 AK096969 BC004974 BC008011 J03040 J03233 NM_003118 |
| | NM_174464 Y00755 Y13714 |
| M2148 | BC001326 |
| M2322 | BC008981 |
| M2433 | BC008686 |
| M2829 | BC003798 |
| M2953 | AK092695 BC006483 BC008492 BC012146 BC012786 BC013861 BC014017 |
| | BC041578 NM_000967 X73460 |
| M2957 | AC006126 AF490256 AK016842 AK093466 BC009217 NM_028954 NM_138568 |
| | XM_218421 |
| O0860 | BC004821 |
| O1036 | BC000905 |
| O1039 | BC003373 |
| O1093 | BC000899 |
| O1117 | BC000138 |
| O1134 | BC003373 |
| O1200 | BC000916 |
| O1336 | BC000991 |
| O2096 | BC008087 |
| O2494 | BC008690 |
| O2602 | BC004850 |
| O2904 | BC002218 |

1. An antibody that binds to an antigen that is differentially expressed between lung cancer tissue and normal tissue, wherein the antigen is encoded by the polynucleotide of SEQ ID. NO. 1.


SEQUENCE LISTING (94 sequences)

SEQ ID NO: 1 - DNA, 2514 bp, Homo sapiens
gtgcagcccgttcgctcacacaaagcccagacgcggagaaaatggcggcaggggtcgaagcggcggcggaggtggcggcgacggagatcaaaatggaggaagagagcggcgcgcccggcgtgccgagcggcaacggggctccgggccctaagggtgaaggagaacgacctgctcagaatgagaagaggaaggagaaaaacataaaaagaggaggcaatcgctttgagccatatgccaatccaactaaaagatacagagccttcattacaaacataccttttgatgtgaaatggcagtcacttaaagacctggttaaagaaaaagttggtgaggtaacatacgtggagctcttaatggacgctgaaggaaagtcaaggggatgtgctgttgttgaattcaagatggaagagagcatgaaaaaagctgcggaagtcctaaacaagcatagtctgagcggaagaccactgaaagtcaaagaagatcctgatggtgaacatgccaggagagcaatgcaaaaggtgatggctacgactggtgggatgggtatgggaccaggtggcccaggaatgattactatcccacccagtatcctaaataatcccaacatcccaaatgagattatccatgcattacaggctggaagacttggaagcacagtatttgtagcaaatctggattataaagttggctggaagaaactgaaggaagtatttagtatggctggtgtggtggtccgagcagacattcttgaagataaagatggaaaaagtcgtggaataggcactgttacttttgaacagtccattgaagctgtgcaagctatatctatgttcaatggccagctgctatttgatagaccaatgcacgtcaagatggatgagagggccttaccaaaaggagatttcttccctcctgagcgtccacaacaacttccccatggccttggtggtattggcatggggttaggaccaggagggcaacccattgatgccaatcacctgaataaaggcatcggaatgggaaacataggtcccgcaggaatgggaatggaaggcataggatttggaataaataaaatgggaggaatggaggggccctttggtggtggtatggaaaacatgggtcgatttggatctgggatgaacatgggcaggataaatgaaatcctaagtaatgcactgaagagaggagagatcattgcaaagcagggaggaggtggaggtggaggaagcgtccctgggatcgagaggatgggtcctggcattgaccgcctcgggggtgccggcatggagcgcatgggcgcgggcctgggccacggcatggatcgcgtgggctccgagatcgagcgcatgggcctggtcatggaccgcatgggctccgtggagcgcatgggctccggcattgagcgcatgggcccgctgggcctcgaccacatggcctccagcattgagcgcatgggccagaccatggagcgcattggctctggcgtggagcgcatgggtgccggcatgggcttcggccttgagcgcatggccgctcccatcgaccgtgtgggccagaccattgagcgcatgggctctggcgtggagcgcatgggccctgccatcgagcgcatgggcctgagcatggagcgcatggtgcccgcaggtatgggagctggcctggagcgcatgggccccgtgatggatcgcatggccaccggcctggagcgcatgggcgccaacaatctggagcggatgggcctggagcgcatgggcgccaacagcctcgagcgcatgggcctggagcgcatgggtgccaacagcctcgagcgcatgggccccgccatgggcccggccctgggcgctggcattgagcgcatgggcctggccatgggtggcggtggcggtgccagctttgaccgtgccatcgagatggagcgtggcaacttcggaggaagcttcgcaggttcctttggtggagctggaggccatgctcctggggtggccaggaaggcctgccagatatttgtgagaaatctgccattcgatttcacatggaagatgctaaaggacaaattcaacgagtgcggccacgtgctgtacgccgacatcaagatggagaatgggaagtccaaggggtgtggtgtggttaagttcgagtcgccagaggtggccgagagagcctgccggatgatgaatggcatgaagctgagtggccgagagattgacgttcgaattgatagaaacgcttaagcagttgccttttttaaacatcgatacgagacctctgaatttgtattttttcttgttaaccattttaatttgttggctggatgtataaagatgtttaaaaaattcagttgctttttggggtaatttgaattacttttttaatgactggggttccatttgactgtttgcattgagattgcaatgtgcgcaattttttttgtagttgtggcatcttgttgacatcgaatatgactttgataataaataccggttcctgaaaaaaaaaaaaaaaaaaaaaaaa

SEQ ID NO: 2 - DNA, 1406 bp, Homo sapiens
gtttcctactcacccaaagccccgcacccgccttttctctctctcctctggcaggatgaggcgtgcaggcctgggtgaaggagtacctcctggcaactatgggaactatggctatgctaatagtgggtatagtgcctgtgaagaagaaaatgagaggctcactgaaagtctgagaagcaaagtaactgctataaaatctctttccattgaaataggccatgaagttaaaacccagaataaattattagctgaaatggattcacaatttgattccacaactggatttctaggtaaaactatgggcaaactgaagattttatccagagggagccaaacaaagctgctgtgctatatgatgctgttttctttatttgtcttttttatcatttattggattattaaactgaggtgatgcatgtaattgtgaatttggaatttgttccaacttaatggcttgcagtaccactttgataaaaatcagcatcaaaacattcctagtgttcaaatactgtggcattttccattgaaaattgctgaattttgcttattttataaatcacattagttaatacagtggtctttgaatactgtttcttaatgactcattttagcccctattttcaggggtagtgagagggtgtggctccactaatttccagtttgtttttctattgtttgccaactgtcagattaaatagcattataatattttgttgtaatcataaatgcaggtttatgtcccatgtaaggaaacttagtgggagagtaacagaatgcctggagagcctgactctgagctcttgaagtagtcagccagtttgtggtaaaatggtaattgaattttcctaactgcatcaactgtaatgatatactcccttctcctcctttatttagttaaaattgtaggctgatttctttttacctacaatcttcctaataatttttgatgataatgacccctcatttctttctgcccaaagacctcattctttaaataaaacttgttattttggcatatttctggtagggcccattgcacatgtgtatcagtatagttattatttcatattaactttatgaattctcttgacttggcttataatagttttatgatttttactacataggtagcacatttatcatttgtgacagaataatgtgaagttaagtaattactgaactttaaatggaaatagtatgcaagaaactcaggcattgaacttgaagataagagtattattgctttaatccagtgtatttgtttatggaaagaaaaacacaaaggcagactgttgagtaaaaaatattaaatattgttaaatattctgtattttggaatttatccatttataggcttcaaaagtaaatttttaaataaaatatattagtcgaaaaaaaaaaaaaaa

SEQ ID NO: 3 - DNA, 1433 bp, Homo sapiens
gcgatagctgagtggcggcggctgctgattgtgttctaggggacggagtaggggaagacgtttgctctcccggaacagcctatctcattcctttctttcgattacccgtggcgcggagagtcagggcggcggctgcggcagcaagggcggcggtggcggcggcggcagctgcagtgacatgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaaaatttgcctccatccttttctcacagcaatgaatttgcaatctgaacccaagtgaaaaaacaaaattgcctgaattgtactgtatgtagctgcactacaacagattcttaccgtctccacaaaggtcagagattgtaaatggtcaatactgactttttttttattcccttgactcaagacagctaacttcattttcagaactgttttaaacctttgtgtgctggtttataaaataatgtgtgtaatccttgttgctttcctgataccagactgtttcccgtggttggttagaatatattttgttttgatgtttatattggcatgtttagatgtcaggtttagtcttctgaagatgaagttcagccattttgtatcaaacagcacaagcagtgtctgtcactttccatgcataaagtttagtgagatgttatatgtaagatctgatttgctagttcttccttgtagagttataaatggaaagattacactatctgattaatagtttcttcatactctgcatataatttgtggctgcagaatattgtaatttgttgcacactatgtaacaaaacaactgaagatatgtttaataaatattgtacttattggaagtaaaaaaaaaaaaaaaaaaaaaaaa

SEQ ID NO: 4 - DNA, 2609 bp, Homo sapiens
agcgctggtaggccttggagaggcgggttaggaagagtggagactgctgcacggactctggaaccatgaacatatttgatcgaaagatcaactttgatgcgcttttaaaattttctcatataaccccgtcaacgcagcagcacctgaagaaggtctatgcaagttttgccctttgtatgtttgtggcggctgcaggggcctatgtccatatggtcactcatttcattcaggctggcctgctgtctgccttgggctccctgatattgatgatttggctgatggcaacacctcatagccatgaaactgaacagaaaagactgggacttcttgctggatttgcattccttacaggagttggcctgggccctgccctggagttttgtattgctgtcaaccccagcatccttcccactgctttcatgggcacggcaatgatctttacctgcttcaccctcagtgcactctatgccaggcgccgtagctacctctttctgggaggtatcttgatgtcagccctgagcttgttgcttttgtcttccctggggaatgttttctttggatccatttggcttttccaggcaaacctgtatgtgggactggtggtcatgtgtggcttcgtcctttttgatactcaactcattattgaaaaggccgaacatggagatcaagattatatctggcactgcattgatctcttcttagatttcattactgtcttcagaaaactcatgatgatcctggccatgaatgaaaaggataagaagaaagagaagaaatgaagtgaccatccagcctttcccaattagacttcctctccttccacccctcatttcctttttgcacacattacaggtggtgtgttctgtgataatgaaaagcatcagaaaagcttttgtactttgtggtttcctctattttgaattttttgatcaaaaaactgattagcagaatatagtttggagtttggcttcatcttcctggggttcccctcactcccttttttgtcaaccccatctgtagcctcttcctctactcaggcagtcgacccgccacgatgagaagtgggaccagcagagggcgccaacttcaggagtccgctttcccaccaggcttcattcacccagtggacctgaactgtttggtagagccacccggcccttccttcctcattgttgtttggtatgcgcacagttcctgtgggactgggccgtgagttttccattggaaagaagttcagtggtcccattgttaactcagcctcaaatctcaactgtcaggccctacaaagaaaatggagagcctcttctggtggatgctttgctccctctgagctgcccatgctggtctggcaaacacacctttctgctttgccttcacaaaagtaatgtgttccctttcccaccccttgcctgaccctcagggagtcagcctgcttccatccatgggtgggaagacttcagcacaaaggaaagactaattcttgtcaggcatttttgaaaaggctgattatgtgtatcaaggtacagcatcgtagggttcccctaaacttgccctgtttttgtttttttagtttgttatccccttactgagcggcctctactaggtggctgtgattaaatgtcccaagcaaggatagggaaggggaatggttgagcctctggagatcattgtaaccaatcctgccagacctgtttggggcagtggggagcaaacctagataaggacctgtttggggcagcagggagcaaaatctcctttaacaaccaagcagttcctcattcacatcaacagagcgaggctgtgataacttaggaggcagcaatcctaatagtccttcagtgcattttagtctgtctccaactggacaccagtaggtagtgtcaagccagagattcggggcagtagataaatgttcattttactgatgcactttagtttttggtctgttacctgttttccagaaatttgtggccttttaggcgggagttaggcgaccaaaccagtgagagccccaatccctgcagttttgtggcttcaagtgtgggtggacagtcctaatggggatctccagctccttcctgtgggctgccacagacagctacccccagaagggtcaatgttgggagtggttgtggctctgagctgctctacagagcttcagtgtgagaggatcgagccattgaaagctcattaccagtaggacataatttttggctctccctattcacaaccagtgcacagtttgacacagtggcctcaggttcacagtgcaccatgtcactgtgctatcctacgaaatcatttgtttctaagttgtgtttattcctggagtgacatgccaccccgaatggctcactttcactgaggatgctgtcctctgatttagctgctgcctccagcctctggcttgagaacttactaaaggcacttccttcctgttaaacccctgttaactctccataaatttggtgattctctgctaggcctaagattttgagttaacatctcttgaagccaaactccaccttctgtgctttttgcttgggataatggagtttttctttagaaacagtgccaagaatgacaagatattaaaaaaaaaaaaaaaa

SEQ ID NO: 5 - DNA, 1291 bp, Homo sapiens
gtgcaaagcgctgtcgggggccgccctagctgccgtcgccgccgccggggctctatggtctctccctagagctttgccgttggaggcggctgctgcggtcttgtgagtttgaccagcgtcgagcggcagcaacatggaggaattcgactccgaagacttctctacgtcggaggaggacgaggactacgtgccgtcgggtggagagtatagtgaagatgatgtaaatgaattagtgaaggaagatgaagtggatggtgaagagcagacacagaaaacccaagggaaaaaaagaaaggcccagagcattccagccaggaagagaagacaaggtggcctctcattagaagaagaggaagaggaggatgccaattcagaatctgagggaagcagtagtgaggaggaagatgacgctgcagagcaggaaaaaggcattggatcagaggatgccaggaaaaagaaggaggacgaactctgggccagcttcctcaatgatgtgggaccaaaatcaaaagtgcccccaagtacacaagttaagaaaggagaggagactgaagagacaagttcaagtaaattgttggtaaaagcagaagagctagagaaacctaaagaaacagaaaaagttaaaatcaccaaggtgtttgattttgctggtgaagaagtaagggtaactaaggaagtggatgctacatctaaagaggccaaatccttcttcaagcagaatgagaaagaaaaaccacaggctaatgttccttcagctctgccatcactccctgccgggtcagggttaaaaagatcaagtggcatgagcagccttttggggaaaattggtgccaagaagcagaaaatgagcacccttgagaagtccaaactggactgggagagcttcaaggaggaagaggggattggtgaagaactggccatccataatcgagggaaagaggggtacattgaacggaaagccttcctcgaccgagtggatcacaggcagtttgaaattgagcgagatctcaggctgagcaaaatgaaaccttgatgttacgggctaaatcaagagcagcttaatcctgtttacaatgtgagctttttgtgcgtctgtgaaatgttttacagtgtttctcatcatctgtttcccagcaaggtcttttttttttctacattgaagttctgtctatgtatcttaatcacaaatggtttcattcactttacttttaaaaatttgtccttaaatgaataaataaaataaaagttggtcctgtgagaggataatgaagatgaaaaaaaaaaaaaaaaa

SEQ ID NO: 6 - DNA, 1648 bp, Homo sapiens
gtccagaggaaggaggccagcaggacctggtgggatagtccctggaaggccagggccctgcacacagactgcaagtcaacacaggagctttgctgtctgaacagccccggtcccgcgttcccagctgtatctactgaagcaggagctgcagcgagccaacatggtttcctcctgtgagctggaattgcaagagcagtccctgaggacagccagcgaccaggagtccggggatgaggagctgaaccgcctgaaggaggagaatgagaaactgcgctcgctgactttcagcctggcggagaaggacattctggagcagagcctggacgaggcgcgggggagccgacaggagctggtggagcgcatccactcgctgcgggagcgggccgtggctgccgagaggcagcgagagcagtactgggaagagaaggaacagaccctgctgcagttccagaagagtaagatggcctgccaactctacagggagaaggtgaatgcgctgcaggcccaggtgtgcgagctgcagaaggagcgagaccaggcgtactccgcgagggacagtgctcagagggagatttcccagagcctggtggagaaggactccctccgcaggcaggtgttcgagctgacggaccaggtctgcgagctgcgcacacagcttcgccagctgcaggcagagcctccgggtgtgctcaagcaggaagccaggaccagggagccctgtccacgggagaagcagcggctggtgcggatgcatgccatctgccccagagacgacagcgactgcagcctcgtcagctccacagagtctcagctcttgtcggacctgagtgccacgtccagccgcgagctggtggacagcttccgctccagcagccccgcgccccccagccagcagtccctgtacaagcgggtggccgaggacttcggggaagaaccctggtctttcagcagctgcctggagatcccggagggagacccgggagccctgccgggagctaaggcaggcgacccacacctggattatgagctcctagacacggcagaccttccgcagctggaaagcagcctgcagccagtctcccctggaaggcttgatgtctcggagagcggcgtcctcatgcggcggaggccagcccgcaggatcctgagccaggtcaccatgctggcgttccagggggatgcattgctggagcagatcagcgtcatcggcgggaacctcacgggcatcttcatccaccgggtcaccccgggctcggcggcggaccagatggccttgcgcccgggcacccagattgtgatggtgagccgtgcgaggcccctcctgtcccccgggctcctcatggggacagtggcagcgggtggggtgacccaggcagacttcacctcccccagacgatgcagatccactctgggctgggcctctgctctttcctgggctgacgtaaagcgttctgctcatttatagatgagagtcgtgccgtgcagaacccagcatgtcacccgtggtgctgctgccatgcggcgcttctgaccaggggtttttgcatgaggccccttgacagggctgctctgggtggtcactttgggtgtgtacagtaaaatacacgtgacataaaattcactattttaaccaaaaaaaaaaaaaaaa

SEQ ID NO: 7 - DNA, 1759 bp, Homo sapiens
cccacgcgtccggcgacatgagtgaacggctgaccacgctggaggccaaggtcctcttgctggaagcagctgagcagccttcaggtccagacaatgacctgccacccccgcagagcacctggatcccagggcctggtggatgagcgggttgtggcaaggccatctggtgagcccagtgtgaaggaagaagaggacaaagccagcgctgcagagggagaaggtgtgcagcagcttcgcgaggccctgaagatcctggcagagagggtccttatccttgagcatatgattggagtccatgatcccctggcctctcctgagggtggctctgggcaggatgcagccctgagagccaatctcaagatgaagcgaggaggtcctcgacctgacggcatcctggctgccctgctgggccccgaccctgcacaaaagagcgcagaccaggctggcgacaggaagtaagagtccctctgtccagtgcgagcccagtgttacagctgaacagcggctgcatcctgaagactccccatgtgggactgggcatcaagcagggaccattgtggaggacattgggagcctggggtccctggggagagatggtgcttccaaggtcagggcccagtgctgactagcaccccgacagagctcttcttttgcccctgccccctacctcccactcccgctggagacggggtctgggtaagctgggtgagctgggtgggctaggtggaggtgggagaataatcacaaccctcaatggctgagcccctgctctgtcagggccagggccagctcaggcactttccctatattgtctcatttaatccttaaaaggtaggtctgtgctccccactcagcctggggaccgatgtgcacatggtacacatgacccggattctggatgtgggggcctggggggtaggttttaacccagacctcactgttgttgaagccatgaccctgaggtgtgtggcccagaggattcctgaggcagctgggctgccgggaggcagggccacctggaaagacatcagggcactacaaaattttgggcctgcctctcttcctctggtcttccttagggctgcgggtaggggtcagcttccaagaggggagtttgggactgtgagggaggagggtctctgagaatgagaaaggcgaagagctttggcgatgccccctaatgtctcccagtcttgctgagcctactggtccttatctgccctcccccatggagaaggaaggaccatttaatgagaccatccatccagaatgggacctacaacctccatcatggccactctttcatgaatatctatatcctaatttgatggacaacacaaatccgtgtccacttaacagagtcagactgcctgtattctaggcttgctaggggccgtggccacacaccttgaggctggggtccaggcaggttacacagaagaactgccccacagccctgaaggcttctctggctggtgcttggtctcttatgacgccctccccccatcctggtgctcctataactgggccaatggccctgacactcagagaacagaaaacaccaccctcgggtatgtctccctgcccagcaggagtgaccatccctaaagttcttggtgcttatgcaaggctgtgacctgaccccactgggccctgtaggctctctctgagttattctctgtctccaccaggaggtgatcagagacctgtgatgttatttcaaataaaggtgctctctccaaaaaaaaaaaaaaaaaaa

SEQ ID NO: 8 - DNA, 622 bp, Homo sapiens
agagacttcctcttcgttaagtcggccttcccaacatggcgcagtctattaacatcacggagctgaatctgccgcagctagaaatgctcaagaaccagctggaccaggaagtggagttcttgtccacgtccattgctcagctcaaagtggtacagaccaagtatgtggaagccaaggactgtctgaacgtgctgaacaagagcaacgaggggaaagaattactcgtcccactgacgagttctatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctgagagtttttgcagaaatggggcagagggacaccctttgggcgtggcttcctggtgatgggaagggtcttgtgttttaatgccaataaatgtgccagctgggcaaaaaaaaaaaaaaaaaaaaa

SEQ ID NO: 9 - DNA, 1940 bp, Homo sapiens
cggacgcgtggggtggtgctgccgttgctcgcagtttcaaaatgcagtgcaggccttagggtctccggctgccacccctcccccagctaggagggggagcgactcatggagcggccgtaagtttgctaactgtggagtcttcactgccaaaatgacatcacattccacctcggcccagtgttcagcatctgacagtgcttgcagaatttcttcggaacaaattagtcagcaggtgcggccaaaactgcagcttttgaagattttgcatgcagcaggtgcgcagggggaagtattcaccatgaaagaggtaatgcactatctaggccagtatataatggtgaagcagctctatgatcaacaggagcaacatatggtatactgtggtggagatcttttgggagatctacttggatgtcagagcttttctgtgaaagatccaagccctctctatgacatgctaagaaagaatcttgttacatcagcttctaataacacagatgctgctcagactctcgctctcgcacaggatcacactatggattttccaagtcaagaccgactgaagcacggtgcaacagaatactccaatcccagaaaaagaactgaagaagaggatactcacacactgcctacctcacgacataaatgcagagactccagagcagatgaagacttgatagaacatttatctcaagatgagacatctaggcttgaccttgattttgaggagtgggacgttgctggcctgccttggtggtttctagggaatttgagaaacaactgtattcctaaaagtaatggctcaactgatttacagacaaatcaggatataggtactgccattgtttcagacactacggatgatttgtggtttttaaatgagaccgtgtcagagcaattaggtgttggaataaaagttgaagctgctaattctgagcaaacaagtgaagtagggaaaacaagtaacaagaagacggtggaggtgggaaaggatgatgatcttgaggactccaggtccttgagcgatgatactgacgtggaacttacctctgaggatgagtggcagtgtacggaatgcaagaagtttaattctccaagcaagaggtactgttttcgttgctgggccttgagaaaggattggtattcggattgttctaaattaactcattccctatctacatctaatattactgccatacctgaaaagaaggacaatgaaggaattgatgttcccgattgtaggagaaccatttcagctcctgttgttaggcctaaagatggatatttaaaggaggaaaagcccaggtttgacccttgcaactcagtgggatttttggatttggctcatagttctgaaagccaggagatcatctcaagcgcgagagaacaaacagatattttttctgagcagaaagctgaaacagaaagtatggaagatttccagaatgtcttgaagccgtgtagcttatgtgaaaaaaggcctcgggatgggaacattattcatgggaagacgagccatctgacgacatgtttccactgtgccaggagactgaagaagtctggggcttcgtgtcctgtttgtaagaaagagattcagttggttattaaagtttttatagcatagttgagtcagtcacagagaaatactaggaggaccaggtcatttatcaaaaaaatcagtattcttagaggcaggggcagaagatcaccaattttgatgccagtctgggccatataatgagatcttagtcttaaaaggatcagtattgagcatcttttataaatgtgacccattgcatatgtttatttgtataagcatatatgaacttttagctaagttttgagggtttcattagtgagaagatactttgttcttccaaattgtgaacccagagggaataatatcaatacaaacatagcaatgcaaaaaaaaaaaaaaa

SEQ ID NO: 10 - DNA, 1699 bp, Homo sapiens
ggcccgggaagactacacacctctttcatggcttcggcgaacacccggcgagttggcgatggcgccggtggcgccttccagccttacctcgactccttacgacaggagctgcagcagagggatccgacgctgctctccgtggcggtggctctcctggcggtgctgctgacgctggtgttctggaagttcatctggagccgaaagagcagtcagagagctgttctcttcgttggtctctgtgactctgggaaaacgttgctgtttgtcaggttgctaactggccagtacagagacacacagacttccattaccgatagttctgccatatacaaagtcaacaataacaggggtaacagcctgactctgatcgacctccccgggcatgagagcttgaggtttcagctcttagatcgctttaagtcttcagctagggctgtggtgtttgtggtggacagtgcggcgttccagcgggaggtgaaagatgtggccgaatttctgtaccaggtcctcatcgacagcatggctctgaagaactcaccgtccttattaatagcctgcaataagcaagatatcgcaatggcaaaatcagcgaagttgatccagcagcagcttgagaaggagctcaacaccttacgagtcacccggtccgcggctcccagcacactggacagttccagcaccgcacctgctcagttggggaagaaaggcaaagaatttgaattttcccagttgccactcaaagtggagttcctggagtgtagtgccaagggtggacgaggggacactggctctgctgacatccaggacttggagaagtggctggccaagattgcctgaggagcatcgctggccacaagcgttggtcatgtgagactgaccctgcagtttgagaaagggcggtggtggtgtggactgataaagagggggcatgtcgttgaagcatttcttgagttcttgaaacagttgtaccacccccacccccgtcagctcctttctcagctagtccttctttcttaattcccatattctttctccctttgctaggtgttaggtgtagcatcatgttagtgtgagacataacagagagggcatcgctttgagagtagggtcgcatctgagaagtccttgtctcctgcctctgcgttggtcccttttcctgggaccagatctgcctgcttgtggaagaagtgaacttgcagttgcccctggtgtaggcataatacagtgtcagttcttagtggagtctggggtgtgaaggtcgacagtgaagacagcttcaaagccacaggcacagctgacagtgtgtccttcacgggctcagtgaccatcccacaccaccctaggttcaaaagctgccgcaaccccgaagagtcaccctcacaaggtggctctaggagccaaatccccagacactcctgggttctagaggctccactggggagagcagggagacagcacaccgctgcctcgggcgtgcgtgagcccaggctgtcccctgtgctacttgatcagctcctgttgtgtcaccccagctagagtgctatggaacaatacaaactgaagatgtccactgtgcattttaaatacatatttattcatgatgctttaaaaaatgttacctttttaaagtgatgctgtcttcaaaaaaaaaaaaaaaaaaaaaaaaaaaaaaaaaaaaaaaaaaaaaaaaaaaaaaaaa

SEQ ID NO: 11 - DNA, 2756 bp, Homo sapiens
aaaaaggtgtaagaatttggagttgctgcagttcaatatgaaggacaacttcagttttgctgccaccagcagaaacattaccagcagccgtccttttgataatctcaacgcaactggcaccaatgagtccgcctttaattgctcacacaaaccatcagataagcatttggaagcaattcctgttctctactacatgatttttgtgattgggtttgctgttaatattgttgtggtctcactgttttgttgtcaaaagggccctaaaaaggtgtccagcatttacatcttcaatctggccttggctgacttactccttttggctaccctccctctctgggcaacctattactcttatagatatgattggctttttggacctgtgatgtgcaaagtgtttggttcttttctgactctgaacatgtttgcaagcattttttttattacctgcatgagtgtcgataggtaccaatcggtcatctacccttttctgtctcaaagaaggaatccctggcaagcatcttatgtagttccccttgtttggtgtatggcttgtctatcctcattgccaacattttatttccgggatgtcagaaccattgaatacttaggtgtgaatgcttgtattatggctttcccacccgagaaatatgctcagtggtctgctgggattgccttaatgaaaaatattcttggctttattattcctttaatattcatagcaacgtgttactttggaatcagaaaacatctgctgaagactaatagctatgggaagaacagaattacccgtgaccaagtcctgaagatggcagctgctgttgtgttggcattcatcatttgctggcttcccttccatgttctgaccttcttggatgctctgacctggatgggtatcattaatagctgtgaagttatagcagtcattgacctggcacttccttttgccatcctcctgggattcaccaacagctgtgttaatcccttcctgtattgttttgttggaaaccgcttccaacagaagctccgcagtgtgtttagagttcccattacttggctccaaggcaagagagagactatgtcttgcagaaaaggcagttctcttagagaaatggacacctttgtgtcttaaatctgttagtgggatgcatgtaatcagcctagccattggtttggaggcccacacaaatgatctttaagtggcatcagtataatacagttctttgctttatctaatctttacttactcccccgagaacaggaagtcaagtagaactgtaaatctttatactccaccagctttcagtgatagtgccttcttttgctggtcctttggcatgagattgtcatatgtgagctagatctataatctagaagtatctgggggaattatcccaacttataattaacaacaaattatgagtggtgatttgacatctcagacttctccctggaaaatgctggcatttcttagtggagttttttgtccattttcatcagatttcttttttcttgaacaaaggccaatttaaacttcttatactatccaaccatatgatatagcatgagaggtgagcactaagtttagcatgatatactcttctatatatgccataggttggtagtggcttattcagtctctaggtatagagtttctccttttaaagaaattgtaagttgtgttccttttccatttcactcaagtatagcttttgtacttattctacagctacacactgagcagatctagaatgtagattaaatcacacatctgtcttagcttattcttgcagttatagaaagtacactatttagtaaaacagaactgcaatgaaaagtattttagtatccacaaaactgaatatacactttgaaaatttttcatccattttgactcttgtttattctattctcttctgatgatttttgaatacaacaacaaaacactgtattatgacactacgtaaaggtcactttttaaatttttaaccttttgaacatggtgctttgatatattcaatgatgacttgagtttaattattcatgcttttgttctgggcttcgtcccaaaatatctctttgaccctgaaaaagagagcattctttaattctttaactttgtaataaagtgcaaactggcatgggaaaaggttatgtcagactggaagtttgatgccttcttgggggtaaacagacccagcaaatggcaagtttggtgtccaacaaggaacttgtcagaacaaagactccctggggagtagtttgaatctgcatttctgggcacagttccagaatgtataagagtctgtgaaggtgatttaaagcaagcccaggtccacagaactcattcttaacacgagtacatctcttacattagaggaatataatacctgaagctgtgttacctaaagtttactcaaacttctcaataaatattaattcagaagttaaagatgtcattctctgcctgtcccatattataccaggtcacctaagaccttcctggattgatgctgacctatgaggtagattcaaagttctgggaacttaacatttctgtcagattccaggcgttttaggttgaagaatcctctcataccccttccttggaaaaccctgatttcatgtattcatgttaatttttagtaaaaacaaatagctaaatatgtaatcagttatgactttgtgttttaagcaattttacacaaaatctcgtaaaaataaaatcattactgggagaccaaaaaaaaaaaaaaaaa

SEQ ID NO: 12 - DNA, 2349 bp, Homo sapiens
ctgaagcccacttctgtagcccttgcttcttccttggttagacgttcgcatcccttccaaattcactccgatctcgccacaatagtggcaaccttgacaccgccttccctgaggagccagccttccttttgtcctgaacttcgcagccctatcatggaggctgtctacctggtggtgaatggggtgggcctggtgctggacttgctgaccttgatgttggatctcaacttcctgctcgtgtcctccctcctggctaccctggcctggcttttggccttcatctacaatctgccacacacggtactgaccagtcttctgcacttgggtcgaggattcctgctttctttgctggctttagttgaagctgtggtccgatttaccttcgggggactgcaggccttggggacggtcctctatagctgctactctggcttggagagcttaaagctactggggcacttggcctcccacggagctctgaggagccgggaattcctgaatcggggcatcctgaacatggtctccaatggccatgctttgctgcgccaggcctgtgacatatgtgctattgccatgagcctggtggcctatgtgatcaacagtctagtcaacatctgcctcatcggcactcagaacttcttctccctggtgctggccctgtgggatgctgtaacggggcctctttggaggatgacagacgtggtggctgctttcctcgctcacatctccagcagtgcagtggctatggccattctcctgtggaccccctgccagctagcactggagctgttggcctcagccgcccgcctgctggccagctgtgtggttttccatctcactggattggtgttgctggcttgcgtgctggcagtgattttgattgtgttgcacccagaacaaaccctgaggctggccacccaagctctcagtcagcttcatgcccgcccatcttaccaccggctttgggaggatatcgtacggctgactcgccttccactaggtctggaggcttggcgcagagtctggagccgcagcctacaactggccagctggccaaatcggggaggagcaccaggagccccccaaggtggccccaggagggtgttctcagccaggatccagccacaggacactcctcctgaagcagaagaggaggtcatcagagctgcaccagctagaggccgagagcaactcaacgaggacgagcctgccgctgggcaggacccatggaagctgttgaaggagcaagaagagcggaagaagtgtgttatctgccaggaccagagcaaaacggtgctgcttctgccctgccggcacctgtgcctgtgccaggcttgcactgagatcctcatgcgccaccctgtttaccatcgcaactgcccgctctgccgccgcagcatcctgcaaaccctcaatgtctacctctgaggactccctccctgcctgcccgtcctctgcgctccacacactgggacagcatcaacactttgcctctgggttctggcctgagtcttctttgcccccgaaggcggcgactctgccaagcctcaagccatatatccaggacgtgtagcttggaatactccggaagactccagtctgggtgggacagagtaacactttctctaaaccctgtgtggggccctgagatgctgagggtggtgagtatgtcactatgcaagggccctgagactctgtggtgggctcttgtgctgcgtctgccagggcccacccagatgcctgcctctagggctgtccttctctgcttctggctcttttgttggagtttttggtcctttcccccaggccacgccacgccacatcagtgttgtttgaggattttggcaactcatgggccttagagtgatcttgattctttttctcttttttgttttttcttggtttggatcttgaacctttatgttcagccagagcctctgccctggtaggtctgggagtagaaacttgcagatgcccttctgcctgtcactgtcttttaaagactcccaacttgaccaggacctggagtcaggagtttatccctgagtacaggggtgcagctacacctggctttgggaccattcaccttcaggcaccccagctccatggggagcctcggaaggaacaaccagatgtctactcattccctacatggcatagaacatagcattcctcctgggctctctccggcagtggagagggcattctgcacttttcacttgggtccaaaggaggggctgggcctcaggggtcatagctccatttagtcggtcggactgcccagtttctatgaataaagttctcttgacaaaaaaaaaaaaaaaaaaaaaaaaaaaaaaaaaaaaaaaa

SEQ ID NO: 13 - DNA, 1922 bp, Homo sapiens
gacagagcgagcgcggcgcggggccaccatgggggcccagctcagcacgttgggccatatggtgctcttcccagtctggttcctgtacagtctgctcatgaagctgttccagcgctccacgccagccatcaccctcgagagcccggacatcaagtacccgctgcggctcatcgaccgggagatcatcagccatgacacccggcgcttccgctttgccctgccgtcaccccagcacatcctgggcctccctgtcggccagcacatctacctctcggctcgaattgatggaaacctggtcgtccggccctatacacccatctccagcgatgatgacaagggcttcgtggacctggtcatcaaggtttacttcaaggacacccatcccaagtttcccgctggagggaagatgtctcagtacctggagagcatgcagattggagacaccattgagttccggggccccagtgggctgctggtctaccagggcaaagggaagttcgccatccgacctgacaaaaagtccaaccctatcatcaggacagtgaagtctgtgggcatgatcgcgggagggacaggcatcaccccgatgctgcaggtgatccgcgccatcatgaaggaccctgatgaccacactgtgtgccacctgctctttgccaaccagaccgagaaggacatcctgctgcgacctgagctggaggaactcaggaacaaacattctgcacgcttcaagctctggtacacgctggacagagcccctgaagcctgggactacggccagggcttcgtgaatgaggagatgatccgggaccaccttccacccccagaggaggagccgctggtgctgatgtgtggccccccacccatgatccagtacgcctgccttcccaacctggaccacgtgggccaccccacggagcgctgcttcgtcttctgagggccgggcacggtcacacggccacccgccccgcgcaccccacgccctgttcacgctcacccagtcacctccccacatcgcacactggggccccgggttcagcctggcctgcccgtgccctggtgaatcacctggctgagcagttcccctggagccccttcgggagcagggctgtgtcccagatgggccacggctgagccttcagagtacgtcctgcctggcacttactggtccttaccagagacgcccagccccatccctgtcctcatgacccctcgtccaccccccacacacactataaggctgagggctgccagcagccccgtctgcccaccattcccggccgtggaccatagtcgggatgtcagcagacacacatgggcagcccaaagctgcaggtgccagggcccaccccagcctcgcctgtcacccccactcccgcctcagggccaggcccaggcctcaccacctgacgctgcatgagacattgacaccagaaagccctcttgggggcactgctccctaccccagggccctggccagccgggagcttggctctcctctggctagagtgggaagagggggctggccatggggccctcccagaacctcagcatttccttccagcccatccaaacactgaggcagccttggggaaccccgagctggggggttggcagcccactgcaccgcctcagggttttggggtcctgggctggggccaccatccctgatggcagaactcccacaaccacatgtatttattcctctgtcctaaaccgtcccctccttccctcacccccagcacagggggattctgagcagtgcctcttgtctgagggacatatcagtgacctcgacgttgcctttagactacagttgtgttagcctcttgcgtattggctttttcagagtcatttatgagcagaaaaaaaaaaagtaaaactttgctaatattaaaaaaaaaaaaaaaaaaaaaaaaaaaaaa

SEQ ID NO: 14 - DNA, 2350 bp, Homo sapiens
cccacgcgtccgcgcggcgccggggttcgcgggagctgcttggaggctcggcggccgggaggaggccggggccacgcttcttggaagctactgagtgacttctttgaagaaccatgaagtcacactatattgtgctagctctagcctccctgacgttcctgctgtgtctccccgtgtcccagagctgtaacaaagcactctgtgccagcgatgtgagcaaatgcctcattcaggagctctgccagtgccggcctggagaagggaactgcccctgctgtaaggagtgcatgctgtgcctcggggccctgtgggacgagtgctgcgactgtgtcggtaggtgcaaccctcggaattacagcgacaccccgcccacatccaagagcaccgtggaggagctgcacgagcccattccgtccctgttcagggcgctgacggagggcgacacccagctgaactggaacatcgtctccttccctgtggcagaggagctgtcacaccatgaaaacctagtctccttcctagaaactgtgaaccagctgcaccaccaaaacgtgtctgttcccagcaacaatgtccacgcccccttccccagcgacaaagagcgcatgtgcacagtggtttactttgatgactgcatgtccatccaccagtgtaagatatcctgcgaatccatgggtgcatccaagtatcgctggtttcacaacgcctgctgcgagtgcatcggtccagagtgcattgactatgggagtaaaactgtcaagtgtatgaactgcatgttttaaagagggggaagaaatgcaaaccaaagcagtaagtcatgaagtgtgcagaaatcttggttctggtatgctaggagtgtgttaagttatatgattgtaactgtgctttttatatctggtgcctattagtgtaggtcttttccattggattcaatggaactttagtcacatgaggatcgggagttcagaggagtcctgggaaaacctgacatgctgacagaaggtgccgtcttcttccagctttccaaacacttctcgttttgaacgtgatagcacaagcctggtacatgtgtggttctcacctgccagttgtagaacactaggtccctatagtcacacatctcttaattgtgccttggctggcttacctgttttgtatgagtaaatattacagtttataattctaacaactcacattcaagccatgctgaaacttaatttcaaaccactttacattggttttagaaagtaaatatttactatattttacaacagaagagttttgcctagggccagcgagctgactcagtggataaaggcgcttgctaccaagcctgataacctgagttccatccccagagcccgtacagtggaaggacaggaccagctgctgggagttgtcctctgacctccagacaggcacagtatcatgcgtggaggtgtgcttgtgtgtgcacacacataactaactgtttttaaaaatataaacctcttacatggtgaaatctaaatctgtcgtgtagctctcacactgacagtggtttggatgttatgtcccctgtccgcctgtagtgctggtgtggtgagacacagagtcgtcactgctctggtatagaagagttttgtctaccaagagtgtcatggcatacctttggaacttcatcaaatgcacttgaggatgacctgggtcaggaagtagccaggtaaaagcagcgggactgtaggcgatgctccattagactccgtgcagagcagcaggtgcacagcatagctgggtgtgcggctgaccaggagagggtctgactccgcaccagcagaacagcagggtctccagcacgtgtgggaagcacgtgggagagggttgaggaaggatgcacagatgtggacagagaagcataaaaatgtcgggaactcctagtagggtccaccttaaaatcgctttatagtctctggctttattactctgtaagattacacttgtttctggatatctgaatccaaataagcatcatattttaagaagctctgtttctgaacttccagggggaaatctgtttaatgtgtttactcctagcatactacagaattttctagctctatagcttcttacctagcgtttccatagtgctgagcttcattactacacgcccttcctagtaataaaattctcaccttcaagcatgaatcaaaaacaaatatctataatacacaggttcaattttatagaattgctattttctctagtgcatatctcattaaaagtaactttttaggaataatctttatatgggtacatattttggtacataaaatagaaaatgttcttaaaaaaaaaaaaaaaaaaaaaaaaa

SEQ ID NO: 15 - DNA, 1782 bp, Homo sapiens
aagttctgtagcttcagttcattgggaccatcctggctgtaggtagcgactacagttagggggcacctagcattcaggccctcatcctcctccttcccagcagggtgtcacgcttctccgaagactggatgactgccatggaggagtcacagtcggatatcagcctcgagctccctctgagccaggagacattttcaggcttatggaaactacttcctccagaagatatcctgccatcacctcactgcatggacgatctgttgctgccccaggatgttgaggagttttttgaaggcccaagtgaagccctccgagtgtcaggagctcctgcagcacaggaccctgtcaccgagacccctgggccagtggcccctgccccagccactccatggcccctgtcatcttttgtcccttctcaaaaaacttaccagggcaactatggcttccacctgggcttcctgcagtctgggacagccaagtctgttatgtgcacgtactctcctcccctcaataagctattctgccagctggcgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatggcctggctcctccccagcatcttatccgggtggaaggaaatttgtatcccgagtatctggaagacaggcagacttttcgccacagcgtggtggtaccttatgagccacccgaggccggctctgagtataccaccatccactacaagtacatgtgtaatagctcctgcatggggggcatgaaccgccgacctatccttaccatcatcacactggaagactccagtgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcacttaaaaaaaaaaaaaaaaaaaaaaaaaaaaaaaaaaaaaaaa

SEQ ID NO: 16 - DNA, 1052 bp, Homo sapiens
gcgcgctctcctagaagtagagggtcgcgcgatgccgctgcacaggtatcctgtgcacctgtggcagaagctgcggctgcggcagggcatctgtgcgcgcttgcccgcccacttcttgcgctcactggaagaagaacggacgccgaccccggtacactacaaacctcacgggaccaagttcaagatcaaccccaagaatgggcagcgcgagcgcgttgaggacgtacccattccagttcactaccctccagagtcccagcagggactgtggggtggagagggcttgattttaggctacagatacgccaacaacgacaagctctccaagagggtgaagaaggtgtggaagccacagctgttcactcgggagctttacagtgaaatcctggacaagaagttcaccgtgactgtgaccatgaggaccctggatctcatcgatgaggcctatggatttgacttctatattctcaagacccctaaggaggacctgggctccaagtttggcatggacctgaagcgagggatgctgctgaggcttgctcgccaggacccccatctccaccctgagaaccctgagcggagagcagccatctatgacaagtacaggagctttgtcatccccgaggcagaggccgagtgggttggcctgacgctggaggaggccctggagaaacagaggcttctggaggaaaaggaccctgtacccctgttcaaggtctacgtagaggagctggtccagcggcttcaggagcaggttctgtccaggcccgcagtggtgcagaagagagccggtgaccacgcctgagcactgggctcagcctcccctgtgggcagtgggcgaaccccacttggcatgtgccgagcacacagttcctgtggtagacttttcagagaacccttctgtgccaggactcgtggaagcctggagcggagcagatgcctgtccaagctggtcaagtccttctagttcttgggctgaaataagtaacagtctgaggcagcctgcgccacagtaaagtgtgttggtttgcttggaaaaaaaaaaaaaaaaa

SEQ ID NO: 17 - DNA, 3417 bp, Homo sapiens
ctctgaccggccggagctgcggagcggctccgctcgctggtttcccggagccgccgcccgctttctccgcccgcggtgagcccgtcaatccccgcacaacgcgctcctccatctgggccatggatggtgttgttacagatcttatagcagtcggtttaaagcggggatctgatgagcttctgtcttctggtgtcattaacggaccttctaccatgaacagctcgacttctgcagctaatgggaatgacaacaagaaatttaaaggagatagacctccctgttcgccttcccgtgttctccatcttcgaaaaattccatgtgatgtcaccgaagcagaggtcatatcattaggtctaccatttggcaaagtaactaatcttttgatgttgaaaggaaaaagccaggcctttttagaaatggcttctgaagaagctgctgttactatgataaattattacactcctgttactcctcatcttcgaagtcagcctgtttatatccagtattccaatcaccgagaacttaagactgacaatctgcctaatcaggctagagcccaagctgcactgcaggctgtcagtgcagtccagtcaggaaacttgtcccttcctggagctactgcaaatgaaggcacattgctacctgggcagagccctgtgctccggataattattgaaaacctattttaccctgtcactctggaagttctccatcagatattttctaaatttggcacggtgttgaagattatcacctttacaaagaataatcagtttcaagccttgcttcagtatgccgacccagtgaatgcgcagtatgccaaaatggctctggatggccagaatatctacaatgcgtgctgcactctccgtattgacttctccaagctcaccagccttaatgtgaaatacaataatgacaaaagcagagacttcactcgcttagaccttcctactggtgacggccagccatctcttgaacctcctatggctgctgcttttggtgcaccgggtataatgtcctctccatatgcaggggctgctggctttgccccagccatcgcatttcctcaagcagcaggtctgtctgtcccagctgttcccggagcccttggtcctctcacacttacctcctctgctgtcagtggaaggatggccattcctggggcaagtggtatgcctgggaattctgttctgctggtcaccaacctcaatcctgactttatcacaccacatgggctttttatcctatttggagtgtatggtgatgtacatcgagtgaagattatgtttaacaagaaagaaaacgccttggtgcagatggcggatgcgagtcaagcccagctagcaatgaaccacctaagtggtcagaggctctatggaaaagtacttcgggctacactgtccaaacatcaagccgtccagctccctagagagggacaggaagaccaaggtctgaccaaggacttcagcaatagtcccctgcatcgctttaaaaagcccggctcaaagaatttccaaaatatcttccctccatcagccacacttcatctttccaacattcctccttctgtcacaatggatgacctgaagaacctgttcacagaagctggatgctcagtgaaggcttttaagttcttccagaaagaccgtaagatggctctcattcagttgggatctgtggaagaagcaatccaggctcttattgagcttcacaatcatgaccttggagagaatcaccacctcagagtttccttctcaaaatccaccatctgaccttcccgtgaaattgtctccttatactggaccacagtttctgtaagatcttcaggcacagactgaagcagctcaagaccatttctgcctcttcacaaaataactcttcgggaatttgatattcgagaacagtcaaaacaagagagagtccttttcttcctctcgttttccccaacagtgtatgatcaaaagtttgctttgcctttgtattgtggtttctatgaaaataactttcggggggaaaaaatcaggaaaatctttttaaaataatttaattccctgaatgagatttaaaaaggacagagtttctttctgtttgggtccagagcagccttacatgacatttctaaactcctttgtactttgagaaatttaaacatacagacttttacaatttcttgatgtaatttaaatcacctacaaccaagtttttttaactttatgattcagaagtttccctgtagaaaatgacattcattcacttataaaaattataccagatatttgcatctattgatagttattatacataactacatggagatatattggaaacaggtttataaacgtgcatgctttctttcagatttcttttttccccccactgtagcacactcctttaaataatgtccatcttctctttttaagaccatttggaaaagcaaagtgttgcagttgtccccagggaaacagaagtgaaattcagcctggatcttttacaatatcagcataattagtcattttaggaaaaaaaatcatgttttaaatttcaaaatgacactgtctagtaatgtaatgtgatcttggaaaattttaaaagaaaaataatcctacttttttataattgttttcagaaaaaaagtttacagtcttaaggaaaaatattcaggtctatcatatggtttgacagattttttaaaagttatttttggtaaggtcttcttttagaaaaaaaaatctcaagggttttttgtaccactataatctctaatactcagaattactgtgtatttacttaatttcttattatgtgccttattatgtgcttaagatacaatagattagaattttatctaaatatctagaaagataaattgtgggcttggtgagcattctgttattttttcctgtcttggattttaagggttttggttgtttttgttttgtttttttctttttttctcgttgcaattttaagtggttttcaataagtaatagtttctactcagatttttggtgctttgggatagtggtgggatagagaagggtgagtggattgggaataaataactcagtgtgcacctgtaggcctctctaggttggctgccgccagccgtcagggaagtctgagctgtagacctgcagactcggatctctgtctgtctgccttcactgcccgttagattttgtctcaggattacttgattccttctcagcactcaagtgagaacctgattttctctgttaatgcgacttcattacagatagcccacatatttggagtatgagaaagtgggttatttttttatattctgaccctcttacttttttggttgaatgtaaaaagtacatattaatgttgcttcaatgattgtgtcatgtaaaattactttttaatatgaaaaaaaaaaaaaaaaaaaaa

SEQ ID NO: 18 - DNA, 2907 bp, Homo sapiens
gcgcaccaccacccgcctgcgccgccgccagccgctggcccgcgactgccctcggaggagctggtccagacaagatgtgaaatggagaagtatctgacccctcagctccctccagttccgataatttcagagcataaaaagtatagacgagacagtgcctcagtggtagaccagttcttcactgacactgaaggcataccttacagcatcaacatgaacgtcttcctccctgacatcactcacctgagaactggcctctacaaatcccagagaccatgcgtaacacagatcaagacagaacctgttaccattttcagccaccagagcgagtcgacggcccctcctcctcctccggcccccacccaggctctccccgagttcactagtatcttcagctcccaccagaccacagcgccaccacaggaggtgaacaatatcttcatcaaacaagaacttcctataccagatcttcatctctctgtcccttcccagcagggccacctgtaccagctgttgaatacaccggatctagacatgcccagttcgacaaaccagacggcagtaatggacacccttaatgtttctatggcaggccttaacccacacccctctgctgttccacagacgtcaatgaaacagttccagggcatgcccccttgcacgtacaccatgccaagtcagtttcttccacagcaggccacctactttcccccgtcaccaccaagctcagagcctggaagtcccgatagacaagctgagatgctgcagaatctcaccccacctccgtcctatgccgctacaattgcttccaaactggcgattcacaacccaaatttacctgccactctgccagttaattcgccaactctcccacctgtcagatacaacagaaggagtaacccggatctggagaagcgacgtatccacttctgcgattataatggttgcacaaaagtttatacaaagtcgtctcacttaaaagctcacctgaggactcatacgggtaggtgatcttcttgctaattctgcgagttctgtaaacagtgtaagcggcattcatccttttaaaaagccaatacgtgtttgatctcttctttcttctgtcaacttttattttttaatggcctaccttttcagcaagggcttgcttggctcaaaattccgttccccaagaaagtctggttccgggaaggctgaggtttccccagtgagaccttgccctgtcaccctcacgtagaattaatgaccctcaaaacgtaaacggcagtgtttactaatggattttaaaactttaatggcaatgctattcctgtcttgaaagaaggaagaaaaccctccttggtaaatcgtcgttgcttcaataaggcccctttttaagctaaaaacttttcccgtcaccatgttaggcaccggtgcttagcacattatcttcagtttcaccagtggcttcaaatatttgtgttttcacctttttgccatgtctgagaagtaggcctaagatctgcagagggtgaaagagctcggccagggtgagggcaggatttgaagcattcctctccagccaaccccgggccttggcttgtctgagaggcctagtccatccttaagtcttccatttcattttcttctgcagcttgcttttaagttcagtctctaattatattttgatgtgttttgttaaattcacagcaacccccttgtaggagctattcattttgaagccgcccttgtatcagaaacacgtttacaatgacgttcagtagcctactttattagagggatccacctacccataatatcttcaagtgtctggatggttactattataattttcttaaagaatagccctgaaggaaaagacagatataaaagtgactgcattccccccctcaataaatacaccctccttacctaaagttcaaagaataagtttgctaataaaagcagtgacctagcaaaatgtattattccatcattgctatggaaaccagagtattcaacagctcctttgaatgggttgttggattagtgtgaggacttgcctctccctaggctggaggttactatggaaattgagttttgataaatgaagtagttatctctaaccactgcattaaaaagtaaaagcttttggacaaaggtcagtggcgaagcagagctctgtgagagtatggtttcagacattgtgtatgcgtgttaggagggagcagtgcttggttatctgaatgcctaacacttcctaaaacccgggtggtagctgtgagctttccaagaatggcttttgaaagaaaaggcatctctcagtttatctgtgataggagtgaaattatttgggcctgaggttgggttgggttttttgtgggcaggtaagcatgaacagaataaatccaaagatgattcaaactccagtctaaaacctcccaaatagagttgacaggtgccagaagagtccatatagaaacactaaagcaacctactaaaatgtccttttgtagactagtgttagggaaacactgaacatggggttttgagctttggatcaagaggagaaacagaagtagtttaggaacatttgagctgttgtttcttctctctcatcagaagtccttgaaactggagataggaaagaggaaaggtattttttgaggttaaagtccccataataaaagaagtgtgcaattgcatataccgacgtaatcaagtgcttattgcaggcacatcgtcaggctatatctaaatagcctttccaccctgtcagggtttactgctctgaatgcttgtacctgggatcctggaacttgacctgctggcagagaaatttaatagtgggtcgtctgaacaatgcccacactaataaaaaaaaaaaaaaaaaaaaaaaaaaaaaaaaaaaaaaaaaaaaaaaaaaaa

SEQ ID NO: 19 - DNA, 2564 bp, Homo sapiens
cgtcctccaggtcgggctgacagaggttgccgtggccgccatattgacttagcggccagggttttgaggggtcagggagacccggacaggtccgcggaactactcggtgggctccggaggaaagccatcatgttaaggatacctataaaaagggccttgataggcctttctaattctcctaaaggatatgttcgcacaactggcacagcagcaagtaacttgattgaagtatttgttgatggtcagtctgtcatggtggaaccaggaaccactgttctgcaggcttgcgagaaggtcggcatgcaaatccctcgattctgttaccatgaaaggttgtctgttgctggaaattgcaggatgtgcctggtagagattgagaaagctccaaaggttgtcgctgcttgtgctatgcctgtaatgaagggctggaatatcttgacaaactcggaaaaatctaagaaagccagagaaggtgtgatggagttcttattagcaaatcacccattggattgtcctatttgtgaccagggaggtgaatgtgatctgcaggaccagtccatgatgtttggaagtgataggagccgatttctagaggggaagcgtgctgtggaggacaagaacattgggcccctagtaaagaccatcatgactagatgcatccagtgtacccggtgcatcaggtttgcaagtgagattgcaggagtagatgatttgggaacaacaggaagaggaaatgacatgcaagttggcacatacattgaaaaaatgtttatgtctgaactgtctgggaatgtcattgatatctgccctgtaggggccctaacctctaagccttatgcctttactgcccggccttgggaaacaagaaagacagagtccattgatgtaatggatgcagtgggaagtaacattgtggttagcacaagaactggagaggtaatgaggattttgccaagaatgcatgaagatattaatgaagaatggatctctgataaaaccaggtttgcttatgatggactaaaacgtcaaagacttactgaaccaatggtcagaaacgaaaaagggcttttaacttatacctcctgggaagatgcactctctcgtgtagctggaatgttacagaattttgaaggcaatgctgtggcagcgattgcaggaggcttggtggatgctgaagccttagtagctctgaaagacttgcttaataaagttgactctgacaacttatgcactgaagagatcttccccactgaaggagctggtacagacttacgttccaattatcttctcaataccacaattgctggtgtggaagaagcagatgttgttcttctagttggtacaaatccacgttttgaggcaccgctgtttaatgctagaattagaaagagctggcttcataatgacctaaaagtggccctcatcggtagtccagtggacctcacttacagatacgaccatctaggagactctcctaaaattctgcaagacattgcttcagggaggcattcattctgcgaggtcttaaaggatgctaaaaaaccaatggtggttttaggcagttctgcactccagagagatgatggagcagcaattcttgcagctgtgtccaacatggtacaaaagattcgagtgacaaccggtgttgctgcagagtggaaagttatgaatattctgcataggattgcaagccaggtagctgctttggaccttggctataaacctggggtagaagcgattaggaagaaccctcccaaaatgctgtttcttctgggagcagatggaggttgtatcacgcgacaggacttgccaaaggattgtttcattgtttatcaaggacaccatggtgatgttggtgctcccatggctgatgttattctcccaggggctgcttacacagaaaagtctgctacttatgtcaatactgagggcagagctcagcaaaccaaagtagcagtgacgcctcctggcttggcaagagaagactggaaaatcataagagctctctctgagattgcaggtatcactcttccatatgatactctggatcaagtaaggaaccgtcttgaagaggtctctcctaatctggttcgatatgatgatattgaagaaactaattactttcagcaagcaagtgagcttgccaagctagtaaaccaggaggttcttgctgacccactcgttccacctcagctaactataaaagacttctatatgacagactcaattagcagagcctcacagacaatggccaagtgtgtcaaagctgtcactgagggcgctcaggcagtagaggagccgtccatatgctgagacatctaccagggaccctttttgctacagacagtaaatggacattgctgtaatcccttataagttaatgttttccaacatgagtctgaatgatgtaatatttaaagttatactatgcttatccgaaaatggtattgtacttatcaaaaatcctggcagtttaaaatgtatgtaattgtgtgcaagcattaactagtttaataaaactatcattttctctttcatgttaaaaaaaaaaaaaaaaaaaaaaa

SEQ ID NO: 20 - DNA, 3360 bp, Homo sapiens
gcgccctgtcaggcgccgccattgttccaccggtaagacccaggtgtgggtccccggcgccatgagcaaacgcaaggcgccgcaggagaccctcaacggcggcatcacggacatgctcgtggaactcgcaaactttgagaagaacgtgagccaggcgatccacaagtacaatgcgtacagaaaagcggcatctgtgatagccaagtacccacacaaaatcaagagcggagcggaagctaagaaactgccaggagtaggaacaaaaattgctgaaaagattgatgaatttttagcaactggaaaattgcgtaaactggaaaagattcgtcaagatgatacaagttcatccatcaatttcctgactcgagttactggcattggatctcaggaaaaatgaagataaactgaaccatcatcaacgaattgggctgaaatatttcgaggactttgaaaagagaattcctcgtgaggaaatgctgcagatgcaggatattgtacttaatgaaattaaaaaagtggactctgagtacattgctacagtctgtggcagtttcagaagaggcgcagagtcgagtggagacatggatgtcctgctgacccacccaaacttcacgtcagaatccagcaaacaggtacctcggaatggataaccaaatgtgatctgtcttacgcagaaggagtattgctttctaattgatggttaggtattttcaccatgtaggttttgtttgtttatggtgctggggattgaactcagggcctcatgcagggactttaccactaagctataaccctaaccctgaaattgactttaatctttatatatagacatgcagacacacacacagagtattgctgcaaattaaacataagtgagggaacaaatgagtgagtgaatctctgtgagaggcatctgctgatgctcctcacaccataaaaagtaactcataactcataaataaagcataactcagtaaagtgagaaggatgaatacctaacatgaaggagatccttggtgacaggtaggcatggggacaagagccactcttgagatgtgaacgcctgtgtgttctcttactgttttccatgataagccagcgtttatagataaaaatatgccacgaacagtaccacataggtcttgcaaataaaaattcataacttgtagcgtgagcgcccacaggaaggaagagtggtgggagctctcagggagacgcacactgactgtcagagagttgtatgtagtacttcgtgcctagccacagggttagggggttgttacagttttcatatgtgtttccaccagctttagttttttgtttttttttttaaaggaatctatactaattcggagtttatcttaaacatagccaaagattgtgtttagagaggacattgggtaaaacatcaagagggacgtccctgtgccatccttccatggtcttgtccactaaaatagaagatcaagaaacccatagtagttttcttatctctctcctgtccccggggctttaccatacttggtttcttactgtaactgcctggttttgtagcacaacacttacatctgtctgatgtcacatgagccaggacacagacacctggcagaattcttcgcacagcagtggatacactggtgtcccctgaatgtcatcctgctgaccccctaagctgactgtgtcctgtgagaggcaaagtgagttacagagcaggattcagtttttttagcatatcctaacatctaaagagatgagattaaagaagatattttcagaattatttgttatcaatatcaatacttagcataaaatacatattttcattgagagaatttttatctgttacagaagggccagagatatggctcagcaagtgaagctgcttgtcattcgggttcagtccctgcacccacatggtgggggagagagctaacactctaaagttgtttctctgcctctgcatgtgtacgcgtgtgtacacttacacactaaataaataaatgcaaataactactgtaaaacttatttaaaaaaaacatataaaggcagatctttaaattttagaatatttttgctttttaaaaagagctaaccgggcagtggtggtgcacgcctttaagcccagcacttgggaggcagaggcaggcggatttctgagttcaaggtcagcctggtctacaaagtgagttctaggacagccaggggtatacagagaaaccctgtctcgaaaaacaaaacaaaacaaaaaaacagctaattctttgctttgttgaggccaaatgaagtttattatcattgaaagcataagtaggtttttttttttttttttaatgaatacaacattctgcctacatgtatgcttgcatgtcagaaaagagtacagggtctcaatgtagatgcttatgagccacctgtggttgctgggaattgaactcaggacctctggaagagcagccatctctccagccccatataagtagtttctaagaaaaagaaattgtttcatttgatggtcagacatggtgggtccttgaatctcatcacttgcactcggaaggtttagcatgagttcagtgttagcccactctacaaagtaaatcccaggcctgacggggtaagaaggagagagagggagaggggggagggggagagagagagggagagggagaacacgaataggaaggtgggcagtagaggcaggacagtcagtgcaagattgcagggcagtttccctggagaatgccgagtagagctgagagaccccaacatgagagcaaccagctctgaagccctgttccctggttataagccccaacatgcacaggttggtatgcacacacatatattaaatgagtaagtaattttggagacttatgtctttaatgatgcacttgtgtctgtgtaggagtgtgtgtgtgagcacaggtgccctcagagaccagaggtctctgttggttccctagaaatatagctacaggcagttgtgagccacctaatgtgaatgctagaaattgaactggggtcatctggaggaacggtaagtgttcttaaccactgagccatctttccaatcccaagtaacttctctttttaaaggaaacagcatgggactcaggacataactcaggtagaatgtttgaatgtgtagcattcacaaggccctgggttaggtccctaagacagggcatgatggtgcggcagcaggttatcctggtctacatattcctaacacagcctggatagaccttctcaaggaaaggaaggaaaaaaagaaacaagtgtagcagacatagcttagtggcagagactaaggccctgggttcactttccaacaccaaaaaaaaaaaaaaaaaaaaaaaa

SEQ ID NO: 21 - DNA, 1153 bp, Homo sapiens
gggagaccatggcaaagaatcctccagagaactgtgagggctgtcacattctaaatgcagaagctctgaaatctaagaagatatgtaaatcactgaagatttgtggactagtgtttggtatcctggccttaactctaattgtcctgttttgggggagcaaacacttctggcccgaggtatccaagaaaacctatgacatggagcacactttctacagcaacggcgagaagaagaagatttacatggaaattgatcccataaccagaacagaaatattcagaagtggaaatggcactgatgaaacattggaagtccatgactttaaaaatggatacactggcatctactttgtaggtcttcaaaaatgctttattaaaactcaaatcaaagtgattcctgaattttctgaaccagaggaagaaatagatgagaatgaagaaattactacaactttctttgaacagtcagtgatttgggttcccgcagaaaagcctattgaaaacagagacttcctgaaaaattctaaaattctggagatttgcgataatgtgaccatgtactggatcaatcccactctaatagcagtttcagaattacaggactttgaggaggacggtgaagatcttcactttcctaccagtgaaaaaaaggggattgaccagaatgagcaatgggtggtcccgcaagtgaaggtggagaagacccgccacaccagacaagcaagcgaggaagaccttcctataaatgactatactgaaaatggaattgaatttgacccaatgctggatgagagaggttactgttgtatttactgtcgtcgaggcaaccgttactgccgccgtgtctgtgaacctttactaggctactacccatacccctactgctaccaaggaggtcgagtcatctgtcgtgtcatcatgccttgcaactggtgggtggcccgcatgcttgggagagtctaataggaagattgagttcaaacgcttaaccttctgttagccaatatataattaatgcatgctactccatgaatttctgcctatgaggcatttgcctccaagtagcctatccttcagaattacttgtaggatattcctctcttcatgttctaataaacttctacatcatcaaaaaaaaaaaaaaaaaaaaaaaa

SEQ ID NO: 22 - DNA, 3674 bp, Homo sapiens
gcccggcggcggctcccggcgtactagtgcggtcgacggctcagataagttccgtggaggcggcggtggcgccgacgaggtgttgggcgacgggagcgggcccggaacttggcgctcagcatgccgacggtggaggagctgtaccgcaactacggcatcctcgccgatgccacggagcaagtgggccagcataaagatgcctaccaagtgatactggatggcgtgaaaggcggcaccaaggaaaagcgattagcagctcagtttataccaaaattctttaagcattttccagagctggctgattctgctatcaatgcacagttagacctctgtgaggatgaagatgtgtcaatacgacgtcaagcaattaaagagctgcctcagtttgccacaggagaaaaccttcctagagtagcagatatactgacccagcttctgcagacagatgactctgcagaatttaacctggtgaacaatgctctgctaagtatatttaagatggatgcaaaaggaacgttaggtggcttgtttagccagattcttcaaggagaagacattgttagagaacgagcaattaaattcctgtccacaaaactcaagacgctcccagatgaggtgttgactaaggaagtggaggaactcatactcactgagtccaagaaggtcctagaagacgtgactggtgaagagtttgttttgttcatgaagatactgtctgggttgaaaagcttacagacagtgagtggacgacagcagctggtggagctggtggcggaacaggctgacctggagcaggccttcagcccctcagaccccgactgtgtggacagactactgcagtgcacgcggcaggctgtgcctctcttctccaaaaatgtgcattctacaagatttgtgacgtatttctgtgagcaagttctccctaacctcagcaccctgaccaccccagtggaaggcctcgatatacagctggaggtattaaagttgttggcagagatgagttcattttgtggtgacatggaaaaactagaaacaaatttaagaaaactgtttgataagttattggagtatatgcctctccctccagaagaagcagaaaatggggagaatgctggtaatgaggagcccaagctgcagttcagttatgtggagtgcttattgtacagttttcatcagttggggcgaaaacttccagacttcttaacagccaaactgaatgcagaaaaactcaaggatttcaaaattaggttgcagtactttgcacggggcctgcaggtttatatcagacaacttcgcttggctctccagggtaaaacaggcgaggccttaaaaacagaggagaacaagataaaagttgttgcactgaaaataacaaacaatatcaatgttttaatcaaggatctcttccacattcctccttcttataagagcacagtaacattgtcctggaaacctgtgcagaaagttgagattgggcaaaagagaaccagtgaagatacaagttcaggttcaccacctaagaagtctccaggaggaccaaaaagagatgccagacagatttataatcctccgagtggaaagtacagcagcaatctgagcaactttaattatgagaggagccttcaggggaaatagaggtggccgaggttggggaacacgaggaaatcgaagtcgaggaagactctactgattgtgacgtcacattcttcagcattgtcatgagattaatatacttaaatctactactcattggattgccggggatgtccctttaaaaggactgctgccttcagctgaaaatgtaatgttctttctacctttgtatgtatgatctacttttgtaaaagaccatggttgtgtccaaggtaaaaccacaacaatatttttggatgctttgtctgcagccttgacttgttttcacaatatcctcactatccagacttcatcttgtgaatcttgcttttacacatcttgatggtttgtcactgagatctgactctattggaaaagtcagtctagcctgtgatctggtgaagatcatcactcttgaaacgttactgttctcctccccattccgttaggtttctgcccaacacatggagaagagttaagagcagtcttaaccttctgctttcattgttttctaaggaagctgctaggcagtgttgtcgcattcctgccaagtcatagaaatgaagcaccagcttctcacagtgtacaggcgatgccagccttttagaaagaacttgctttgcattgccccttcagtcttctccatattttgggatacagctttgaacacagggtcttattatttgaataggagtacatgtgcataatatacgtacagacataagcatatgttgtgtgtgtatatatgcatgctatggaacttgagtgtacaaccaatcaatacattaaattctcggaaggggtcctctgacatagcgattatccctttgaggctgaatccattactgacttggtatctaggttttcactccagtagggaggaatttcagtcagctgaacttacagattattattgttgtttaaaactaaaaaacaaaggctgtttttacagagacaaatttaaagacccttgttggtgaaatacagccagatctggtgtacatacaggttcccacaggatctgctagtgtaatgtaggatttggagacttaataaccagggctacccaggagtgtgacttggtgacatagtaccataaaagttgctcactccgcttgcttttgccactttcaaattttaacttctcaggttattaatcagattattgtgtaagttagccaatagtctttagattaaggcaacagacgggaggttcgtggagtgtctcatgttgggcatttttagtagcccagactctgttcttcatttgaatgtttcacacatttttgttcacagttaatcttccaagtttactattcaaatcagaaattcagatgacatttctagtggtttgctgttttggttttttatgttttttggtttttttgaggtttcatttcttacacaggtgtcttcatcaccatcacttctacactgggggaaaaacaatctcctttgtgagaatcactgcacgtatttatggcgaaaatatttctgaaagtctagagtgatacaagtgagcacaagaagttggtcagcttgcctatggagtgctggcaataaactctgaacattccacaagcctgagctgaacctaggctcccttggaagctgaacagacataggaacatgggattgccagctgaatcctgtgaccatctagttatggacctcagagatagatcagcatggcctaaagccatttcaagtacaaaaatgaattggactatagcaacataaatttgaacacagtaacttttcctctttgcctgagggcatttgtaaactcggcagaagtaacttgacctcatgtctactgcagagctcactgcattcacccctcctcatcctttgcttccttccccttgcctagtcagtagttcataacttagtgttccttttgcttcagaaatctgaaagaaaatcttcatgccatgaatctttttcttccgcaaggacacctgtgtctcccttgtttaaataaatgtaaatgctcccttatgcttttgaaataaatttccttttgtaaaaaaaaaaaaaaa

SEQ ID NO: 23 - DNA, 1551 bp, Homo sapiens
gcgattcttcccgcagagttgtgaagcgaaaggcttacaattaaaaggaagaaaaaaaaataaagataattcgggagtacaattgacaaagcgtgtgggtcgctcagcctccagcagtaactgctgatctccagttcttggagggttccggtgagaagaacgcccctactgcggtactgaggaagcggcaggaggagatgcggcccctggacatagacgaggtggaagcgcctgaggaagtggaggtgctggagcccgaggaggatttcgagcagttcctgctcccggtcatcaacgagatgcgcgaggacatcgcgtctcttatacgcgagcacgggcgggcgtacctgcggaccaggagcaagctgtgggagatggacaatatgcttatccagatcaaaacgcaggtggaggcctcggaggagagcgccctcaatcacgtgcagcacccgagtggcgaagccgacgagagagtgtcggagttgtgcgagaaggctgaggagaaagccaaggagattgcgaagatggcagagatgctggtcgagctcgtctggcgaatagagagaagcgagtcttcttgaaggaggagatcggtggttcacagctgaggactgtagtcaactggtaaggatgaactgacaccttgaggaaactgaaacagcttgtcattttctctgttttgtttttgtatgttttttacccccatgtaacagtctcccttatggtcagtgattgatgacctcgatatggatttagattatcaaatgtgtttggttttggaaatataacttttgtcaaagaaatactctcagaagagaaatggggcttaattaagttgtttttgtggtcacgtttattcttgttacttcgctgtgtttttgaaatgttgggcatggcctcgtattttgctgttacctttgtgacctgattgttttttggaacacgtcaagacgtgggatcagaatcttccaactttagaggtgcaatggaagacactacgctacttggttgagcctggtgaagaatgtattaatgagactgctttgcataaaactgggaagaaagagaagacagttggagatggaagatggttttgtatatattttggaactttagttcctctgtgagacgaaagaggagagctatgttttgtgtcacattgtctgatatatattgtgtaacctgtcaggtgagttgatttagacaacatagctgaccttttatgacaaggcagtttgaatagggactattgtaataccctcacacattataggggcatcagagaatggcatggaagagacagtctacagagagctttaagaggccggagaaaggaaaagacattatcagggcctggaaagtctcttccagttcatcagggtagtagacctgtcagattggaggtcaaagtcaaaagtataataagtgttaatttgccttgaaaaacaaagacctaaaaagccttttttaagaaataaattttttgttcacatgaccaaagcccctcctgtgtgtgttaataaaagaatcataaacaaaaaaaaaaaaaaa

SEQ ID NO: 24 - DNA, 1579 bp, Homo sapiens
cggtctccggcgccagctacgccgctgccgctgtcactatggcccattacaaagccgccgactcgaagcgtgagcagttccggaggtacttggagaagtcgggggtgctggacacgctgaccaaggtgttggtagccttatatgaagaaccagagaaacctaacagtgctttggattttttaaagcatcacttaggagctgctactccagaaaatccagaaatagagctgcttcgcctagaactggccgaaatgaaagagaagtatgaagctattgtagaagaaaataaaaaactgaaagcaaagcttgctcagtatgaaccacctcaggaggagaagcgtgctgaataggattcttctcagtttgaaagacaatgaaaaatggttttgtatgacttgaatagtttgtatagtatataatcttttctgaacagatgctatagaactcttttaatatgtttaattcacctatcacactctgttaaaaacacatagaatcatcaataaaaactcaatataactttctttgggtcttaaagcaggagaatccaaagtaaatcctgaacaaaacctaaacacagccatctaactcattaccttaaaagacattctgtttattagtctgattaggaatgatggcactggttgtattttagccaagacagtttagcatggagctattccttggtgcagttcaggatatgaacacaggtacagtcattctttgaaggtgacactgttctgtatattccctataggcagctggagagatctgtgtgacacaagatgcttttgtacgggttcccatgaatcttctgctcttgtttgtgtgacatggaacaaataacttctttgccaccactttgccttagataactgtgtgtgtgtgtgccagtttgaactctgacaccacattttccttctatgcaatcatgcctgtctgataatcttgcattgctttcctctgagctttagtgggtcctagttgcacactggcctttctgtgctgtttttcaatttgcctaataatagcagttaccctgattgtaatttatgtaactttaaacaggatcacactgtaccccctgcctgccttatttgcttactgagcacaggacagaggcaatatacaactctgggttcacacacaagctgagatgagaagaggaatgagccatatattggggaaaatcatagtttgtaggtataattatatagtgcttttctccctcaaagtatttttctagccttgaattcattttatcttcattatccctgtgaagtaggtgggacaagtataaggggaagaggggtgctgaatttttaggccaaagactgatattaatacaaatcactcactaactgtagagccttgggcattatcagtgaactactctgagatttactgtcttcatctgtttaatgagtagaatgtccgtgatgcctacctcacagggttgttgtgagggtcaaatgagaatgtatgtgaaagatttgtaaatggtaaagcactatattcttgtttgttaaaaaaaaaaaaaaaaaaaaaaaaaaaaaaaaaaaaaaaaaaaaaaaaaaaa

SEQ ID NO: 25 - DNA, 657 bp, Homo sapiens
gggacctggagcaacgggctgcaggacggctacgggaccgagacctactcggacggaggtggttggacgcggactagcgctgtcgaagcaggctgctggagggaggagggaggcccatctgctcagtgagagcccaggaatctcgtctttcagtggctgcatcgttttcaccattagttgagggaatcgatctgtgccttcattctaagatgccaccgcattcggggcagagccggggccggaagccagggagctgcctgctgctgctgctgctgctgctgctgctgctgctgctgctgctgtaagatggtttctgtgcagggaaccttggccggctctgcagctgcccgcctgcctggactctccgatatccactcctcagtgcacctgacacgcatggagccggtcctttcctggaagccagaccccaaacaaactggcttccccgaccagtccactcccatgtgggagcttatcctcagaggcactgggtcctctgcctccctcgggcggctcgcctgtttcaggcatggatgcctgggaagggagtgagacgcagcaatgacttgtgcttctgccgagaataaaaatcctgagcgtacgtgtgctctagtcctccccaaaaaaaaaaaaaaaaaaaaaaaaaaaaaaaaaaaa

SEQ ID NO: 26 - DNA, 1255 bp, Homo sapiens
tgccaacttaactgagattctgagtgtgcaaggataacccccgtcatggccccgggtgccaaggaaatcccttttagcctcctcttggcagacttggccagccttccccagtgacagcctggctctttggattcaagctgcctggtcagactgcaagggtcatctgtacaaagaaggaagcaacagtgccaggttccagcgtgcgttccaagttccacattctgtgtgcagactcccaacgccagagcaaaggcaggcccctggcaggggactgctgagaaagtggaactccagagacactcctgaaattgtcctaagtcaaaggagccaaacggtgtacgagctccctctctgccgtcccctctgagagcagccatggccctgagtgatgtcgatgtgaaaaagcagattaagcacatgatggctttcattgagcaggaagccaatgagaaagcagaggaaatcgatgccaaggctgaggaagagtttaacattgagaaaggacgcctcgtgcaaacccaacgactgaagattatggagtattatgagaaaaaggagaagcagatagagcagcagaagaaaatcctgatgtccaccatgaggaatcaggcgaggctgaaagtcctgagagcccgaaatgacctcatctcagatttgctcagtgaggcgaagctgagactcagcaggattgtggaggacccagaggtctaccaggggctgctggataaactggtgctccagggtctgctccgactgctggaacctgtgatgattgtacgctgccggccacaagacctcctcctggtggaggctgctgtacaaaaagccatccccgagtacatgacaatttcccagaaacatgtggaggtccagattgataaagaggcatacctggctgtgaatgcagctggaggtgtggaggtctacagtggcaatcagagaataaaggtttcaaataccttggaaagccgactggatctctcagccaagcaaaagatgccagaaatacgaatggccttgtttggtgctaacaccaacagaaagttctttatataagcctctgggaagtgaagctagtgttgaaccactaaaccataaaagtttaagttttttggggaaaggaaactagtagtgtttcctcctctttgatgctctgatactgttctgtttttcttcacgaaataccctttggatagctaaagtgttaactttgaaataaagcccaaattaatgctcagaaaaaaaaaaaaaaaaa

SEQ ID NO: 27 - DNA, 1851 bp, Homo sapiens
ggcacgagggagagaactagtctcgagttttttttttttttttttgttagtgaacaacgtctctttattttggggaggagcccggcgggacagtagaagtaaacccttgcctgttaattgaactggccgaggtcctgcggggagtgtggggtgggacgtgaagtggcagctcctcagtgacaaggccactatgtgctatacgcatgcgctgtttcttcagcgccagctccgcccccgccgcagcgaggcgggtaggttccgcccgcgcgcactacgccctgacgtcagcgtcgcgcgcagcgcagtgacgaaatcggctggtttatattggcgcggcccagacggcagaggtctctgcggcgcggtcctcggagacacgcggcggtgtcctgtgttggccatggccgactacctgattagtgggggcacgtcctacgtgccagacgacggactcacagcacagcagctcttcaactgcggagacggcctcacctacaagtgcgggcctatggggctcacctgggcgaagagaggggcggcaccctggggaaaaggccagatgggggcttccgagagccgccttgggcgtgggcgtgggggacaggtttcgggaattggccacccacctccggggctcactttgagacatctggagcggtgcttggggtacggagaagcatctcagtagtgcttggaatctcaggccggagattccatgctccagacacatggaattgtcagggtgccaggagcactgatgacagctcggtggcagaaaaggtcccctgtacccatttccaccttgtgctgtgcctgtgacacacagactctgtcagtgccactctctgctcacatgcgttacgggttgattggtacatggggatggagtctcatgctgccctaagctgagagctttactcccaattcactcccttccctcgcagtgactttctcattctccctgggtacatcgacttcactgcagaccaggtggtgagtatgatcaggaattgggtcctgaacatcaaggtgagctaaggtgctactatcacaatactaccaatcaatactcatcattaataatcataatggctatagcaataaaactaggaatagccccctttcacttctgagtcccagaggttacccaaggcacccccctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttataaaaaaaaaaaaaaaaaa

SEQ ID NO: 28 - DNA, 2291 bp, Homo sapiens
ggcacgagggttgtacttcgcagctctctggacattaattacctcctgcaccttccatttgggggagggtgaggaagaaggcactcacctacactgagcgaagaagtcctatcagcgtggaagagaagtttaagaatgaaccactgaatgtttcgcttttatttttaaatttactttggcacttaaaaaacctcatttccagatttctggacaatctcggctgcagacatctaagcctaaccttttggtatcctgagttttttccctctccttctccttcctcacgcccaccccgcttctctcgcacacggtcatctttacatatcaagagaaagcaaaaaggttccaagaggtacttttctttttagaaaatcaccttttctcccctaattcttctgcagaagaaaagaggtttcgaaagagggaaaaggaaaaaacaagccggctacggtatcagccggtcagcccaggtggaaaacgagagtgaaagtagggcttaacggggagcgcaccgcctctttcgaaagccgctgggccaccaccagtgcgtgagccttggatccctcaacgtattgcgagacgccggtgtatagcccggacctgtgccccaacatgatcgccgctcaggccaagctggtttaccagctcaataagtactacactgagcgctgtcaggcgcgcaaggcggccatcgccaaaaccatccgagaggtctgtaaggtggtctcggacgtgctcaaggaagtggaggtgcaggagcctcgcttcatcagctccttgagcgagatcgatgcccgctacgaggggctcgaggtcatttcgcccaccgaatttgaggtggtgctctacctaaaccagatgggcgtcttcaacttcgtggacgacggctcgctgcccggctgcgcagtgctcaaactgagcgatgggcggaagcggagcatgtctctctgggtcgagttcatcacggcgtcgggctatctctcagcgcgtaagatccgctcgcgtttccagacgctggtggcccaggcggtggacaagtgcagctatcgggatgtggtcaagatgatcgcggacaccagcgaggtcaagttgcgcatcagggagcgctatgtggtgcaaatcactccggcgttcaagtgcaccgggatctggcctcgcagcgcggcacagtggcctatgccccacatcccttggcccggccccaatcgggtggccgaggtcaaggccgaagggttcaacttgctctcgaaggagtgctactcgctgaccggcaagcagagctcggcagagagcgacgcctgggtgctacagttcggggaggcggagaaccgcctgctgatgggcggctgccgaaacaagtgcctctcagtgctgaagactctgcgggaccgccacctggagctacccggccagccgctcaacaactaccacatgaagacgctgctgctgtacgagtgcgagaaacacccacgagaaacggactgggacgagtcgtgcctgggcgaccggctcaacggcatcctgctgcagctcatctcctgcctgcagtgccgccgctgccctcactactttctgcccaacctcgacctctttcagggcaagccccattcggccctggagagcgctgccaagcagacctggaggttggccagggaaattctcaccaatcccaaaagcctggacaaactatagggtgctggggactgcttgaaaagcgacacaaacgggcgtgctctctcagacacacaactcagctataaacagcagaaactctggacacaaacttttatgtaagtcacctgaaataggaatccggcagaagaccttcattaattaagaagcaaacaaaaagagagcaacccaaccaaaacaaatcacattcttgcacaaaagtgatcgttttcttccaaacaatgtgaatttaaaaggtcacacaaaagaagcaatcgggctccgccaccacaaaatgaaacccaaggtacattttcaaatcaatgtatagtagttccccccccttttccttctttccccctatcctttttctctccaccccatcccccccccatttcgttttgcttttggttgcctgaatgttgtcaccaagtgaaaaaattatttaactatatgtaaaatttctcttttaaaaaaaagttttactgatgttaaacgttctcagtgccaatgtcagactgtgctcctccctctcctgaacctctaccctcaccctgagctgtcttgttgaaaacagtaataaaaacattactttacattgtaaaaaaaaaaaaaaaaaa

SEQ ID NO: 29 - DNA, 2143 bp, Homo sapiens
tatgctccttccttccccgccttccttgtctccccgactgcgctcgttcctgtggccatcccgcagcgccagtccaggtgccatggctgctatgtacctccccggcctgcggcttagccggcacgggctaaggccctggtgctggtcaccgtgccgtagcatccaaaccctgcgtgtgctcagtggagatatgagtcagctgccggctggagttcgagactttgtggcgcgcagtgcgcatctgtgccaaccagagggcatccacatctgtgatgggaccgaggctgagaacactgccatactggccctgctggaagaacagggtcttatccgcaaactccccaagtataagaactgctggctggcccgcacagaccccaaggatgtggcacgggtagaaagcaagacggtgattgtaactccttcgcagcgggacacagtgcctctcctggctggtggggccagggggcagctgggcaactggatgtccccagatgagttccagagagctgtggacgagagattcccaggatgcatgcagggccgcattatgtatgtgcttccattcagcatgggtcccgtgggctccccactctcccgcattggagtgcagctcactgactcggcttacgtggtggccagtatgcgtattatgacccgcttggggacacctgtacttcaggccctgggagatggtgacttcatcaagtgtctgcattcagtgggccagcccctgactggacatggggatcctgtgggccagtggccgtgcaatccagaaaaaaccctgattggccacgtgccagaccagcgggagatcgtctccttcggcagcggctatggtggtaactccttgctgggcaagaagtgctttgccctgcgcatcgcctctcgcctggccagggatgagggctggctggcagagcacatgctgattttgggcatcaccaaccctgcagggaaaaagcgctacgtggcagctgctttccccagtgcctgtggcaagaccaatctggccatgatgagacctgcattgccgggctggaaagtggagtgtgtgggggatgacatcgcctggatgaggtttgacagtgaaggtcaactccgggccatcaaccctgagaatggcttctttggggtggcccctggtacctctgctgccaccaatcccaatgccatggccacaattcagagtaatactctcttcaccaacgtggctgagaccagtgatggcggtgtgtactgggaaggcattgaccagcctcttccgcctggtgtcaccataacctcgtggctgggaaagccctggaaacctggtgacaaggaaccctgtgcacatccaaactcgcgcttttgtgtcccagctcgccagtgccccatcatggacccagcctgggaagcaccagaaggtgtccctattgatgccatcatctttggaggccgccgacccaaaggggtaccactggtgtacgaggccttcaactggcgtcatggggtgtttgtaggtagcgccatgcgctctgagtccactgccgctgcggagcacaaaggaaagaccattatgcatgatccctttgccatgcggcctttttttggctataactttggacgctacctggaacactggttgagcatggagggacaaaaaggtgcccggctgcctcgtatcttccatgtcaattggttccggagagatgaagcaggctgcttcctgtggccaggctttggagagaatgctcgtgtgctagactggatctgccgaagattagaaggagaagacagtgcccaagagactcccattgggctagtaccaaaggaaggagccctggacctcagtggcctcagcgcagtggacaccagtcagctgttctccatccccaaggacttctgggagcaggaggttcgtgatattcggggctacctgacagagcaagtcaaccaggacctgcccaaggaggtgttggctgagctcgaggctctggaaggacgtgtacaaaaaatgtgacctgaggctctaggctagcaagagcacagctcccccatttgggtagagaacccccaggctcacagaaaacatgaacaatctgacattaaaatgtgtgagtgttggaaggccaaaaaaaaaaaaaaaaaaaaaaaaaaa

SEQ ID NO: 30 - DNA, 797 bp, Homo sapiens
cgctccccgccagcccgcgtccgcaactcaccagctccggaattcgtcccgtggccctagcccgcgcttccacagccggctgggaacggcggcggcgcgggctccaggtacagcgcctctccgggcgagccgcgccgctcccgcgagtagcaggaggcgtccggtcgcagactcccttcgaggcgcttcctgtccggtgagcgtcgaacgactgaagccgcggcccatagtgccttgcgatggcgggtaggcgtgtgtaggcggagccagggccggaagtagaacggtggcggcggcggtgactctggcagctcgggactcagtgcaagtacagagactcagccatggttctgcagaggatcttcaggctctcctctgtccttcggtcagcagtctctgtgcatttgaagaggaacattggtgttacagctgtggcctttaataaggaacttgatcctgtacagaaactcttcgtggacaagataagagagtacaaatcaaagcgacaggcatctggaggacctgttgatattggcccagagtatcagcaagatctggacagagagctttataagcttaaacaaatgtatggtaaaggagagatggatacatttcctaccttcaaatttgatgatcccaaatttgaagtcatcgacaaaccccagtcctgaggaacatacaaaatccatgtggtaatttgtcatgaattagttgtacaactaatcaaaaaattcaaataaacattcatttcacagttaaaaaaaaaaaaaaaaaaaaaaaaaaaaaaaaaaaaaaaa

SEQ ID NO: 31 - DNA, 461 bp, Homo sapiens
aaaaccaggagagcatggagctttttcgctggtccggcgcggtctcttgcgttcctgtggcgggcacctgactccccatgcccgagtactgtcctcaggcggaacggcgtctgcagcgaggagcggggttccagcaggctcggctgacgcggtccctggcatttcccgtgctggccctgggtcgccctcgatgctgcttcccgctgggagcctccttctgacggcggaaaggagcttcacctggggcggccggcggggaacggagaagaatccaccggggaagtggacggggctggctcccgggcctggcgctgcggctctcgggccgtcagacctcccgcgggttgcgtcactttcctcggttttctagacagcagttgtggggttgaatccttgccgtggttttgtaagaattaaatgagataatgaacgcgaaaaaaaaaaaaaaaaaaaaaaaaaaa

SEQ ID NO: 32 - DNA, 2176 bp, Homo sapiens
agggggtgcaagggtccacgggacggacggacgggcgggcgggcactgctctctatatgcgcggcgtgtgcctcggccctgacgggtggggcttcggaaagatccgtggggacggaggccgtggttgtgattgaacgggctgcctccggacccctcccctatcgccaacgctgagccgaaaccatgcatgggcgcctgaaggtgaagacgtccgaagagcaggcagaggccaaaaggctagagcgggaacagaagctaaagctctaccagtcagccactcaagctgtcttccagaagcgggaggcaggcgagctggatgaatccgtcctagaactgacaagccagattctgggagccaaccctgattttgccaccctctggaactgtcgcagagaagtgctccagcagctagaaacccagaagtcccctgaggagttggctgctcttgtgaaggcagaactaggcttccttgagagctgtctgcgtgtgaaccctaagtcctatggcacttggcaccaccgctgctggctgctgagtcgcctgcctgagcccaactgggcccgggagctggagctgtgcgctcgcttcctcgaggccgatgagcggaactttcattgctgggactatcggcgatttgtagctgcacaggctgctgtcgcgcctgcagaggaactagccttcactgacagcctcatcacccggaacttctccaactattcttcctggcattatcgctcctgcctcttgccccagctgcacccccagccagactctggcccccagggacggctccctgaaaatgtactgctgagagagctggagttggtgcagaatgccttctttactgaccccaatgatcagagtgcctggttctatcaccgctggctcctggggcgggcggagccccacgatgttctgtgctgcctgcatgtgagccgggaagaggcctgtctgtcagtctgcttttctcgtcccctgatagtgggctccaagatggggaccttgctactcacggttgacgaggcacctttgagtgtggaatggaggactccagatggcaggaaccggcccagccatgtctgggtatcccgggattggtggggcaggattagggtgggacagtcagaaaagcagtgaggtagcatgtttccttgaccagtgctctagctctgtgacctgcctgctgcctctctcaatgaccacttgccccagcacacgtttcgtgtcatctggacaggaagcgatacccagaaggagtgtgtgcttttaaaaggtgatgctgtctaaagctccccttgcctcgccagccccccagccttctctctcctctccccacagtgtcccatccaactacttaacctttcccctaggtcaccaggagtgctggtgccgagactcagccactgatgaacagttgttcaggtgtgagctgtccgtggagaagtccacagtgctacagtccgagcttgagtcctgtaaggagctgcaggaactggagcctgagaataaatggtgcctgctgaccatcatcctcctgatgagggcactggaccccctcctctacgagaaagaaacactggagtacttcagtaccctcaaagctgtggacccgatgagagcagcctacttggacgacctgcgcagcaagttcttggtggaaaacagtgttctcaagatggagtatgctgacgtgcgtgtgctgcacctggctcacaaggatctcacagtgctctgccatttggaacaactgctcttggtcactcatcttgacctgtcccataatcgtctccgagccttgcccccagccctggctgctctgcgctgtctcgaggtgttgcaggccagtgataatgtcctggagaacttggacggcgtggctaaccttccccggctgcgggagctgttactatgtaataaccgcctccagcagtctgctgcacttcagactcttgcctcctgccccaggctagtcttcctcaacctgcagggcaactcgctgtgccaagaagagggcatccgggagcgtttggctgagatgctaccgtccgtcagcagcatcctcacctaggaggccccatctcaccctcaccctttaacttattgggactgaataaagactggagaggcccctctaggctgccaagctaccattaaaaaaaaaaaaaaaaaaaaaaaa

SEQ ID NO: 33 - DNA, 710 bp, Homo sapiens
gtatatatgcgtatgtatgtatacatatgtgtgtatatatacgtatgtatgtatacatatgtgtgtatatatacgtatgtatgtatacatatgtgtgtatatatacgtatgtatgtatacatatgtgtgtatatatacgtatgtatgtatacatatatgtgtatatatacgtgtgtatgtatacatatatgtgtatatatacgtgtgtatgtatacatatatgtgtatatatacgtgtgtatgtatacatatatgtgtatatatacgtgtgtatgtatacatatatgtgtatatatacgtgtgtatgtatacatatgtgtgtatatatacgtgtgtatatatatacacatatatacgtatatatgtatatatatatacacagttgaatcagtgggattaatacctataatctctggttttcaaaggtaatatggaatatttgacacttggtaaaaggtgaactacctttgtagtgaatcttttcctcttggtagcatcaacactggggataaatcagaaccattctgtggaatgaaatgtttctcaagagcctataatatagtagatagtgcatattaagatgtctggctgggcatggtggctcatgcctgtaatcccagcactttgggaggctgaggcgggaggatcacttgagcctagaagttggagactaacctggcgagaccctgtctcaaaaaaaaaaaaaaaaa

SEQ ID NO: 34 - DNA, 2113 bp, Homo sapiens
gcaggcttgaatggtcccttatgctgtgctctctgtcctgccgctggatgctgtggagggtgctcaggcaagcttggaagttgtgacatggtggatgcattgacctatgatgatgtgtatgtgaacttcactcaagaagaatgggctttgctgaatccttcacagaagagtctctacaaagatgtgatgttggagacctacaggaacctcaatgctgtaggctacaattgggaagacagtaatattgaagaacattgtgaaagctccagaagacatggaaggcatgaaagaaatcatactggagagaaaccctacgaaggtattcaatatggtgaagcctttgttcatcacagtagtctacaaatgcgtaaaataatacatactggagagaaacgctacaaatgcaatcaatgtgataaagcctattcacgacacagcattctacaaatacataaaagaacacatagtggagagaaaccctatgaatgtaatcaatgtggtaaagcctttacacaacacagtcatctcaaaatacatatggttacacatactggagagaaaccttacaaatgtgatcaatgtggtaaagcctttgcattccatagtaccctccaagtacataaaagaacacatactggagagaaaccctatgaatgtaatcagtgtagtaaagcctttgcacatcattgtcatctccgagtacataaaagaatacacactggagagaaaccttacaaatgtgatcaatgtggtaaagcctttgtaggtcagaatgatcttaagagacatgaaagagttcatactggagagaaaccttacaaatgcaatgaatgtggtaaagcctttgtatgtaatgccagtctccgtacacataaaacaacacatactggagtgaaaccttatgaatgtaagcagtgtactaaatcctttgcctctcatggtcaacttcaaaaacatgaaagaattcatactggagagaaaccttacaaatgtgatcagtgtggtaaagcctttgcctctcatgataaatttcaaaaacatgaaagaattcatactggagagaaaccttacaaatgtaagcaatgtactaaatcctttgcctctcatgataaacttcaaaaacatgaaagaattcatactggagagaaaccttatgaatgtaagcaatgtactaaatcctttgcctctcataataaacttcaaaaacatgaaagaattcatactggagagaagccttacaagtgtgatcaatgtaataaagcctttgtatatgaaagttatttacaagttcataaaaaaacacatactggagagaaaccttacaaatgtaatgaatgtggtaaagcctttgcacgacacagtcatctcaaagtgcataaaataacacatactggagagaaaccttacaaatgtaatcaatgtggtaaagcccttgcatatcatagtacactccaagtacatcaaagaacacatactggagagaagccctatgaatgtgagcaatgtggcaaagcctttgcaaatcaaagttatttccaagtacataaaagaatacatactggagagaaaccctacaaatgtgatcaatgtggtaaagcctttgtaggttcaagtgatcttaaaagacatgaaagagttcatactgggagagaaaccttacaaatgtgatcaatgtggtaaagccttttcataccattgtcatcctcgaatgcataaaagaacgcatactaaagagtaaccctatgaatataaccaatgtggtaaagccttacagtgatcttgaaaggtgtggtggactaaatatacttggcccatgacaagtggcagtagtagggagtgtggcctttctggcatagatgtggtgtttgagaaagtctgtcactatatgcttggaccttgagatccacacagggcagaagagtcttctggcttctgatcaagatgtagaactcttgtctcctccagcaccatgtctgcttggacactgccattcttttcgccttgatgataatagactgaacctctgaatctggacccaattaaatgtcctttataagagttaaaaaaaaaaaaaaaaaaaaaaaaaaaaaaaaaaaaaaaaaaaaaaaaaaaa

SEQ ID NO: 35 - DNA, 2326 bp, Homo sapiens
ggcggcgaagcgggggcacgccctcgcacacgcagagataaattgtgctcccatgacctttatttggaaagtgcctgcgggcctaaaattggcctttgtcccaccgagtacactcagcactgtactttaaaccggataaactgggctgtctggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaaaaaaaaaaaaaaaaaaaaaaa

SEQ ID NO: 36 - DNA, 1946 bp, Homo sapiens
ggcggcaaaaacaaaaatattaaaaaaaaggagagagggacaaaaaaaagaaaacccgaaagtttcagctgcattgctgcggaacgttttttagccagaaggcgaaagaagaaatcggaagtgacgtcgcggccaaaacaagcccgggcttggaggcctgtactgaagctggcctcagatgggaaggcccgactcgctgtctgctgtcgtcggtggtcgcgagaccttgcactctcaccgggtcggcctccagcccctgtgcccgggatccgctcgccgcggatgagcgagagtttcttcctgggacttttcgggcacagctggccggtggcgacagaacggactttctttcctgcaagagtctcccctccagcgggagacagcgggctcctgtctcgggacgccgggacacctgtcgcctatttttaaatatccagattccaagaacacactggatactgctcttacaaaaccaagaggaaatcatgaagaaatgttttagttattgaaactacagttgaaatcatggatacatcaacaaatctggatattggagcccagcttatcgtggaagagtgtcccagcacttatagcctaactggcatgccagacattaaaatagaacatccactggacccaaattcagaagaagggtcagctcagggtgttgccatgggaatgaaattcatattgcctaaccgatttgatatgaatgtgtgttctcgatttgtgaagtccttaaatgaagaagatagtaaaaatattcaagatcaggttaactctgacctggaggtggcatctgtcctatttaaagctgaatgcaatatccatacatctccttctccgggaattcaagtaaggcatgtctacaccccctctacaacaaagcatttctcacccataaaacagtcaaccactttaaccaacaaacacagaggaaatgaggtctctaccacacctctgttagcaaattctttgtctgttcaccagttggctgctcagggagagatgctctatctggctactcgtatcgaacaagaaaatgttatcaatcacacggatgaagaaggatttactcctctgatgtgggctgcagcacacgggcaaatagctgtggtagagttcctacttcagaatggtgctgatccccaacttttaggaaaaggtcgagaaagtgcactgtcgttggcctgtagtaaaggctacacagatattgtcaaaatgctgcttgattgtggagttgatgtaaatgaatatgattggaatggaggaacacctctgctttatgctgtacatggaaatcatgtgaaatgtgtaaagatgctcttagaaagtggggctgatccaacaattgaaactgactctggatataattctatggatctagctgtagccctaggctatagaagtgttcaacaggttattgagtcacatttgttgaagctgcttcaaaatatcaaggagtagacacagtcatcagaaaatgtctgcccttttgtttacttcttggtccttataaatgatagttttgtttacttataaatttttacctcagttgcaatatttactggtttttagtaggttttaataaatatttctctgagtaattcactggtttataataaatgtaatactctttttataactatgttttactgtatatttaaaattataaattaatgttttcgtggcatgtaaatttttatggtacagatagttatcatcagtctttgtatcaagtgctgtaatttgacattttcagaaattattctaccctagtcatcttcactcgtgtattaagtcattcactttatatagggtttgctataaatccctagaaaaaaattgttcttattgttgaataaaaaagtgcacagtgtgattgtttacaaaatgatattataaataaataaaatacttcttctgtcaaaaaaaaaaaaaaaaaa

SEQ ID NO: 37 - DNA, 1806 bp, Homo sapiens
gagaagagggcagagccgtgcatggggctgctccccaggacctgagcaggaacctggagttttcagagctgcctgatcattgctacagaatgaactctagcccagctgggaccccaagtccacagccctccagggccaatgggaacatcaacctggggccttcagccaacccaaatgcccagcccacggacttcgacttcctcaaagtcatcggcaaagggaactacgggaaggtcctactggccaagcgcaagtctgatggggcgttctatgcagtgaaggtactacagaaaaagtccatcttaaagaagaaagagcagagccacatcatggcagagcgcagtgtgcttctgaagaacgtgcggcaccccttcctcgtgggcctgcgctactccttccagacacctgagaagctctacttcgtgctcgactatgtcaacgggggagagctcttcttccacctgcagcgggagcgccggttcctggagccccgggccaggttctacgctgctgaggtggccagcgccattggctacctgcactccctcaacatcatttacagggatctgaaaccagagaacattctcttggactgccagggacacgtggtgctgacggattttggcctctgcaaggaaggtgtagagcctgaagacaccacatccacattctgtggtacccctgagtacttggcacctgaagtgcttcggaaagagccttatgatcgagcagtggactggtggtgcttgggggcagtcctctacgagatgctccatggcctgccgcccttctacagccaagatgtatcccagatgtatgagaacattctgcaccagccgctacagatccccggaggccggacagtggccgcctgtgacctcctgcaaagccttctccacaaggaccagaggcagcggctgggctccaaagcagactttcttgagattaagaaccatgtattcttcagccccataaactgggatgacctgtaccacaagaggctaactccacccttcaacccaaatgtgacaggacctgctgacttgaagcattttgacccagagttcacccaggaagctgtgtccaagtccattggctgtacccctgacactgtggccagcagctctggggcctcaagtgcattcctgggattttcttatgcgccagaggatgatgacatcttggattgctagaagagaaggacctgtgaaactactgaggccagctggtattagtaaggaattaccttcagctgctaggaagagcgactcaaactaacaatggcttcaacgagaagcaggtttattttttccagcacataaaagaaaaataatgtttcggagtccaggactggcaggacaggtcatcagatactcagaggctgtatctctgccctgccaaccttgacaaatggcttccaatgttaggtttgctacaagatggttactggagctctagctgcctattttgtgtttagggaagggaaaatggaggaaaggggagaagagcaaagggcgcttttaaagagctttcccaaaagctccacccaatgacttctgcttccatctcactaaccacccacccctacctggaatggaggctgggagatgtggcttatttgctgggtacgtgactatccctaataacaaaggggttctgacactaagacattaggggagaatgttgggtaggcagccagcactcttttaccagagggcctcctggtgtttggattttgatctcaatgtgtaaaatgacagagatgtaacaagctcatagggtatcaataaaaaaaaaaaaaaaaaa

SEQ ID NO: 38 - DNA, 1980 bp, Homo sapiens
ctttctttgatcctaatgatgcatcatgccaggaaattctttttgatcccaaaacttcagtttcagaattatttgccattttgagacagtgggttcctcaggtccaacaaaacattgacattattggaaatgagattcttaagagaggttgcaatgtgaatgatagagatggattgacagatatgactcttttacattatacctgcaaatctggagctcatggtattggtgatgtggaaacagctgtaaaatttgcaactcagcttattgacctgggagcagacattagtttgcggagtcgctggacaaacatgaatgctttgcattatgctgcttattttgatgtccctgaacttataagagtgattttgaaaacgtcgaaaccaaaagatgtggatgccacttgcagtgattttaattttggaacagctttgcatattgcagcatacaacttgtgtgcaggtgctgtgaagtgcctcttggagcagggagcaaatcctgcatttaggaatgacaaaggacagatccctgctgatgttgttccagacccagtagatatgccgttagagatggctgacgccgcagccactgctaaggaaatcaagcagatgcttctagatgcggtgcctctgtcatgtaacatctcaaaggccatgctcccaaattatgatcatgtcactggcaaggcaatgcttacgtcacttggcctgaagttgggggatcgtgttgttattgcaggacagaaggttggtacattaagattttgtggaacaactgaatttgcaagtgggcagtgggctggcattgaactggatgaaccagaaggaaaaaataatggaagtgttggaaaagtccagtactttaaatgtgcccccaagtatggtatttttgcacctctttcaaagataagtaaagcaaaaggtcgaaggaagaatataacacacactccttctacaaaagctgctgtacctctcatcaggtcccagaaaattgacgtagctcatgtgacgtcaaaagtaaatactggattaatgacatcaaaaaaagatagtgcttctgagtcaacactttcattgcctcctggtgaagaacttaaaactgtgacagagaaagatgttgccctgcttggatctgtcagcagctgctcctctacatcttctttggaacacagacagagctaccccaagaaacagaatgcaatcagcagtaacaagaagacaatgagcaaaagcccttccctttcatccagagccagtgctggtttgaattcctcagcaacatctacagcaaataatagccgttgcgagggggaactccgcctcggagagagagtgttagtggtaggacagagactgggcaccattaggttctttgggacaacaaacttcgctccagttctaggatattggtatggtatagagcttgaaaaaccccatggcaagaatgatggttcagttggaggtgtgcagtattttagctgttctccaagatatggaatatttgctcccccatccagggtgcaaagagtaacagattccctggataccctttcagaaatttcttcaaataaacagaaccattcttatcctggttttaggagaagttttagcacaacttctgcttcttcccaaaaggagattaacagaagaaatgctttttccaagtgagtattaagaagatttagaaaaacacctttcaagatgaacatgatgggattctgtgaagaaagaaaagtggagtgctgtatcttttgcattgcttggtagagttttaacacgtaggagaaaaggtgtgataagtgtattgagaagcgttcttcctccctcctttgcaagcgcaattaaaaatgtcaccagtgactaaaactagtgcactgcatatattttatcttcatagaggtcagaactgttaataaaagttaaaggaatttaaaaaaaaaaaaaaaaaaaaaaaaaaaaaaaaaaaaaaaaaaaaaaaaaa

SEQ ID NO: 39 - DNA, 1260 bp, Homo sapiens
aaaaagacaagagggaaagtatttctcttgattcttggttaaatttgtttaataataataatatcctaaaaaaaaaaaaaaagggcggccgcagggccatgctagccttgcgcgtggcgcgcggctcgtggggggccctgcgcggcgccgcttgggctccgggaacgcggccgagtaagcgacgcgcctgctgggccctgctgccgcccgtgccctgctgcttgggctgcctggccgaacgctggaggctgcgtccggccgctcttggcttgcggctgcccgggatcggccagcggaaccactgttcgggcgcggggaaggcggctcccaggccagcggccggagcgggcgccgctgccgaagccccgggcggccagtggggcccggcgagcacccccagcctgtatgaaaacccatggacaatcccgaatatgttgtcaatgacgagaattggcttggccccagttctgggctatttgattattgaagaagattttaatattgcactaggagtttttgctttagctggactaacagatttgttggatggatttattgctcgaaactgggccaatcaaagatcagctttgggaagtgctcttgatccacttgctgataaaatacttatcagtatcttatatgttagcttgacctatgcagatcttattccagttccacttacttacatgatcatttcgagagatgtaatgttgattgctgctgttttttatgtcagataccgaactcttccaacaccacgaacacttgccaagtatttcaatccttgctatgccactgctaggttaaaaccaacattcatcagcaaggtgaatacagcagtccagttaatcttggtggcagcttctttggcagctccagttttcaactatgctgacagcatttatcttcagatactatggtgttttacagctttcaccacagctgcatcagcttatagttactatcattatggccggaagactgttcaggtgataaaagactgatgaaagtcatccctcactgttagtaaggaagcagtatacatcaatgggaacagggcccatggaaatgtacaggagtttccctattttggtgttcagcttgaaaaaggacttgtcagaatcaactgtgtcatcaaaatttaagtaatgtgcattgaaaataaggttgatcatgggaatatgcagaatttccaatgtatttttaaatacaaataaaattgtaatttagaatttttaaaaaaaaaaaaaaaaaaaaaaa

SEQ ID NO: 40 - DNA, 1779 bp, Homo sapiens
agcggaggtgtgagtgagtggatctgggtctctgccgttggcttggctcttcccgtcttcctcccctcctccctccctgactgaggttggcatctagggggccgagttcaggtggcggcgccgggcgcagcgcaggggtcacggccacggcggttgacggctggaagggcaggctttcttcgccgctcgtcctccttccccggtccgctcggtgtcaggcgcggcggcggcggcgcggcgggcgcgcttcgtccctcttcctgttccctcactccccggagcgggctctcttggcggtgccatcccccgacccttcaccccagggactaggcgcctgcactggcgcagctcgcggagcgggggccggtctcctgctcggctgtcgcgtctccatgtcggataaccagagctggaactcgtcgggctcggaggaggatccggagacggagtccgggccgcctgtggagcgctgcggggtcctcagcaagtggacaaactatattcatggatggcaggatcgttgggtagttttgaaaaataatactttgagttactacaaatctgaagatgaaacagaatatggctgtaggggatccatctgtcttagcaaggctgtgatcacgcctcacgattttgatgaatgccggtttgatatcagtgtaaatgatagtgtttggtaccttcgagctcaggacccggagcacagacagcaatgggtagacgccattgaacagcacaagactgaatcgggatatggatctgagtccagcttgcgtagacatggctcaatggtgtcactggtgtctggagcgagtggctattctgctacgtccacctcttctttcaagaaaggccacagtttacgtgagaaactggctgaaatggagacatttcgggacatcctgtgccggcaggttgatactctccagaagtactttgatgtctgtgctgacgctgtctccaaggatgagcttcagagggataaagtcgtagaagatgatgaagatgacttccctacaactcgttctgatggagactttttgcacaataccaatggtaataaagaaaaattatttccacatgtaacaccaaaaggaattaatggcatagactttaaaggggaagcaataacttttaaagcaactactgctggaatccttgctacactttctcattgtattgaattaatggtaaaacgggaagagagctggcaaaaaagacacgatagggaagtggaaaagaggagacgagtggaggaagcgtacaagaatgtgatggaagaacttaagaagaaaccccgtttcggagggccggattatgaagaaggtccaaacagtctgattaatgaggaagagttctttgatgctgttgaagctgctcttgacagacaagataaaatagaggaacagtcacagagtgaaaaggtcaggttacactggcccacatcattgccatctggagacaccttttcttctgtcgggacgcatagatttgtacaaaagccctatagtcgctcttcctccatgtcttccattgatctagtcagtgcctctgacgatgttcacagattcagctcccaggttgaagaaatggtacagaaccacatgaactattcattacaggatgtaggtggtgcactgtatactcccaaagttctggtctcgtttttatattaaaaaaaaatacaaatgttttattgtaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 41 - DNA, 1659 bp, Homo sapiens
ggagagagcaggacacagctatggatgccgccaggagaggagatacacagccagtgatgtggaccaccggatggctgttgctgctgccgcttctgctgtgtgaaggagcgcaagccctggagtgctacagctgcgtgcagaaggcggacgatggatgctctccgcacaggatgaagacagtcaaatgtggtcccggggtggacgtctgtaccgaggccgtgggagcggtagagaccatccacgggcaattctctgtggcggtgcggggctgcggttccggaatcccgggcaagaacgaccgcggactggaccttcacgggctcctggccttctttcagctacagcagtgctccgaggaccgatgcaacgccaaactcaacctcactttgcgaggcctcaaccctgcaggcaatgagagtgcatatgagcctaacggtgcagagtgctacagctgtgtgggtctgagccgcgagaagtgccagggctccatgccgccggtcgtgaactgctacaacgccagtggccgtgtctacaagggctgcttcgatggtaacgtcaccctgacggcagccaacgtgaccgtgtccttacctgtccgaggctgcgtccaggacgagacctgcacccgggatggggtgacgggtccaggattcacactcagcggctcttgctgtcagggcccccgctgtaacgccgaccttcgcaacaagacctatttctcccctcgaatcccacccctagtcctgctgccccctccaaccaccgcagccccatccactcgggcccagaactcctccagcacgacctctacagcagccccaaccacgaccacctccatcatcaagcccaccacagcccaagccagccacacttctccccatgaaatggatctcgaagtcatacaggaagagggggcgtcgttgagtggaggtgctgcgggccatggaggtactgcgggccatggaggtgctgcgggccaccaagaccgcagcaatatggagaagtatccaggaaagggtggggcccagatcccagctaaaggaggctctggcactctagggtcctggttgtctgcagttctgttgactgtggttgctggcgcgatgctgtgaatgtctcatctcgaaaagtccatctcactttgtttccctggccccgtggtaccaactctttccatttctcacttgactggactggctccgcccccatccttcagcattctcagttccgactgcactggtttgcagcttcggaaaacagtcctctgttgtaaatattccgctcgggtggccctacttttttgatgcggccacagcattccccctgatggtgaccaggacagagggaagagacgtctactggctgagagaggcccagagagtccacggcaagcctcctcttcccgttttcctgaccaggctggaagatgaccaggcaggtagacaatggatccatcctccgagcactgtgcttgcctggcacattgtgcggaaatctggtcgcctgtcttccttaggagactgtgaacaactctacaacagggtcttgtctctggcctctctatgtgttctgtctggcacaggaaggtgtcaataaagatttagttactttgtatagtgagttaactaaaaaaaaaaaaaaaaaaaaaaaaaaaaaaaa

SEQ ID NO: 42 - DNA, 1843 bp, Homo sapiens
ggccgcccttttttttttttctaattctcttttcctctcttcctcttgtcctccagcctcagcagatgagacttttacgcatcatcttcagcgactgagaaaactcattaaaaagcgctctgaactgtatgaagctgaagagagagccctcagagttatgctagaaggagaacaagaggaagagaggaaaagagaattagaaaagaaacaaagaaaagaaaaagagaaaattttacttcagaaacgtgaaattgagtccaagttgtttggggatccagatgagttcccacttgctcacctcttggagcctttccgacagtattatctccaagccgagcactccctgccagcgctcatccagatcaggcatgattgggatcagtacctggtgccatccgatcatcccaaaggcaacttcgttccccaaggatgggtccttcccccgctccccagcaacgacatctgggcaactgctgttaagctgcattagtaaagatgctccaggagtgtggtccagccagcgctctttccagctgtaaatattagcgatggtgccatcttttgctgtagactaaactgcaacttctaaattccatgtggcattcccctaccctgaagttatgctttccttctgtgctctgtgctggccagaggtgcctcttgaatcagattaatgtggtttttcaggaaaggacttaggtgaactgaggtttttaccacaggcagtgaatgaccttggttcaccaaatttgcctctgttttgaggggcttggtccagagtgacttgttaatttactctaacttccttgtgtgttgatgggtaagtacactcaaacactgaatacaggtgtgtgatgggtagatttcacagcccttctactaatagtgagtgtgaaggcaagcttgatgcaaaacctcctgacctttcctacctgaagagccctttgacttctaggaagaaaggtcaaaaatgttatcttcagttgtgttaatcccagttttagtgcagcttaggaggctgctagttaggaagatggcagtggctgtaggctgggttgccagaaaagatggtggcctagtcttattattcagatggagaacttagaaaacctgaagagtacccaaattggattgtattttaatggacaatggctgtattttttccatgttagaaggatcctaatgaaagcacctgttatttttaagtttctaagggtctagttgttcagaatccccaaggatatttccctaacctcactcagtcacattgtaggagccagtgtagctatggaattatcttaggaactcaagcttctaaaactatccatgtagtcaaatctaggggaaaaagcaaataaaaatagtaaaatttggccgggcacagtggctcacgcctgtaatcccaacactttgggaggccgaggcgggccgatcacgaggtcaggagatcaaggccatcctggctaacacggtgaaaccctgtctctactaaaaatacaaaaaaatattagctgggcgtaggtggtgcacacctgtagtcccagctactggggaggctgaggcaggagaatggtgtaaaacccaggaggcagagcttgcagtgagccgagatcgcgccacggcactccagcctgggagacagagcaagactccgtctcaaaaaaaaaaaaagtaaaatttattttttatattcattaataaatgctgttgtgcttggagatgatcactcatataacatgtcatttttttggttctgttttggtttggtttttgccaattattttgttatatttccaaaaaactaaataaaaatcatttttattttttaaaaaaaaaaaaaaaa

SEQ ID NO: 43 - DNA, 2112 bp, Homo sapiens
cggacgcgtgggcggacgcgtgggtcgtaaaggcaaaaaggccaaagcctcactgtttcaattgtggttctgaagaacaccaaatgaaagattgcccaatgcctcggaatgctgctcgaataagtgaaaagagaaaagagtatatggatgcctgtggagaagcaaacaatcagaatttccagcagcgataccacgcagaagaagtagaagaaagatttggaagattcaagccaggagttattagtgaggaacttcaagatgcactaggtgtgacagacaagagtcttccaccttttatctatcggatgcgccagctagggtacccaccagggtggctcaaagaggctgaattggagaattcggggcttgcactctatgatggaaaagatggcactgatggggaaacagaagttggagaaatacaacagaataaaagtgtcacttacgatctctcaaaattggtcaactatcctggttttaatatatctactcccagaggaattccagacgaatggaggatctttggttccataccaatgcaggcatgtcagcagaaggatgtgtttgccaattaccttacttctaacttccaagcgccaggtgtgaagtctggcaacaagaggtcttcatctcactctagcccaggtagtccaaagaagcagaagaatgaaagcaactcagcgggatctcccgccgacatggagctcgattcagatatggaggtaccacatggttctcagagcagcgaaagttttcagtttcaaccaccattacctcctgacactcctccactcccccggggaactcctccacccgtcttcacccctccactcccaaagggcaccccgccgctgactcccagtgactcaccccagaccagaacagcatctggagctgtggatgaggacgcactgactctagaagaacttgaagaacagcagaggcggatctgggcagctcttgagcaggccgagagcgtaaacagcgactccgacgttcctgtggacacacctttaactggcaattccgttgcctcatcaccttgtccaaatgagctagacctccctgtcccggagggaaaaacatctgaaaagcagacgctggatgagcctgaggtaccagagatttttacaaagaaatcagaagctggacatgcctccagtccagactctgaggtgacatcactttgtcagaaggaaaaagcagagttggctccggtaaacactgaaggtgcccttcttgataatggcagtgtcgtaccaaactgtgacatcagcaatgggggcagccagaagctctttcctgcagacaccagtccttcaacggccactaaaattcatagccctatacctgacatgagcaaatttgcaactggaatcacgccatttgaatttgagaatatggcagaatctactggaatgtacctcaggataagaagcttattaaagaactcaccccgaaaccagcagaaaaacaaaaaggcctctgaataatggcttgacttagcactgagagctatttaataactttgttccgttaattagtactaattaagtggatagatagaatggttttcctgtttgtccctcccatgtttaaaaatctatccaaggttcatgttccaaagtcaagcctattttaaagaaagactgagctcactagttcagtatattttattctcactgacaaaacttggggggagatgtgaatatgacctggtttagagagggtttgttaaggtttatactatttttggattgtgactatccgtcgagagtgatggtttttatctgtcttttgtacattgttttccctttctacattttgctaattatcctgtatataagtttaatatatcactttttaaaagaaaaaattctaccattttaaattcatgtttcaactcctacaaccaaatgagaaaaatcagggatgagcagctttatcccatttggggtatttttgtaagtgatttacatgtgtcaattttagtaatacttttacttttttgtaacttcatccttcatatatgcttgctatacaggtatgttcatctttgtgtacagaggtttaataaattagttttcatatacataaaaaaaaaaaaaaaaa

SEQ ID NO: 44 - DNA, 3228 bp, Homo sapiens
cggacgcgtgggtcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaaaaaaaaaaaaaaaaaaaaaaaaaaaaaaaaaaa

SEQ ID NO: 45 - DNA, 2710 bp, Homo sapiens
gcggctcagaccccctccccggcccgcatctgtgcagctttccgggcgatgccagaatagatgccggggcaatgtcccgccgcaaacagggcaacccgcagcacttgtcccagagggaactcatcacgccagaggctgaccatgtggaggctaccatcctcgaggaagacgagggtctggagatagaggagcctagcagcctggggctgatggtgggaggccccgaccctgatctactcacctgtggccagtgtcagatgaacttcccgctgggggacatcctggtttttatagagcacaagaagaaacagtgtggaggcctgggcccctgctacgacaaggtcctggacaagagcagtccacctccctcctctcgctctgagctcaggagagtatctgagccagtggagatcgggatccaggtcacccctgatgaagatgaccacctactgtcacccacgaaaggcatctgtcccaagcaggagaacattgcaggtaaagatgagccttccagctacatttgcacaacatgcaagcagcccttcaacagcgcctggttcctgctgcagcacgcacagaacacgcatggcttccgaatctacctggagcctgggccggccagcacctcgctcacgcccaggctcaccatcccgccaccgctcgggccggagaccgtggcgcagtccccactcatgaatttcctgggggacagcaatcctttcaacctgctgcgcatgacgggccccatcctgcgggaccaccctggcttcggtgagggccgcttgccaggtacgccaccgctcttcagcccaccgccacgccatcacttggacccacaccgcctcagtgcagaggagatggggctcgtggcccagcaccccagtgccttcgaccgagtcatgcgcctgaaccccatggccatagactctcctgccatggacttctcccggcggctgcgagaactggccggcaacagctccacgccgccgcccgtgtccccaggccgtggcaaccctatgcaccggctgctgaaccctttccagcccagtcccaagtccccgttcctcagcacgccaccgctgccacccatgcctgcgggcacaccgccaccgcagccgcctgccaagagcaagtcctgtgagttctgcggcaagaccttcaagttccagagcaatctcatcgtgcaccggcgcagccacacgggcgagaagccctacaagtgccagctgtgcgaccatgcgtgctcgcaggcgagcaagctcaagcgccacatgaagacgcacatgcacaaggcgggctctctggctggccgctcagacgacgggctctcagctgccagctcccctgagccgggcaccagcgagctgccaggtgacctgaaagcggccgatggcgacttccgccaccatgagagcgacccatctctgggccccgagcctgaggacgacgaggacgaggaggaggaagaagaggagctgctgctggagaacgagagccggcctgagtcgagcttcagcatggactcggagctgggccgtggccgcgagaacggaggtggtgtgccaccgggggtggcgggcgcaggggctgcagctgcggctctggcggatgagaaggctctggccctgggcaaggtgatggaggacgcagggctgggcgcactgccgcagtatggggagaagcggggcgccttcctgaagcgtgcaggcgacacgggtgatgccggagctgttggctgtggggacgcgggtgcaccgggtgcagtgaacgggcgcggcggggccttcgcgccaggcgcagagccctttccagctctcttcccacgcaagccagcaccgctgcccagccctgggctcggtggtcccgcgctgcacgcggccaagcgcatcaaggtggagaaagacctggagctgccacctgccgccctcatcccatctgagaacgtgtactcgcagtggctcgtgggctacgcagcatcgcgccacttcatgaaggacccattcctgggcttcacggatgcgcgccagtcgcctttcgccacatcgtcggaacattcctctgagaacggcagcctgcgcttctcaacgccacccggggacctgctggacggcgggctgtccgggcgcagtggcacggcgagcgggggcagcacacctcacctgggtggtccgggtcctgggaggccgagctccaaggagggccgccgcagcgacacatgtgagtactgcggcaaggtcttcaagaactgtagcaacctgacggtgcaccggaggagccacaccggcgagcggccttacaagtgcgagctgtgcaactacgcgtgtgcgcagagcagcaagctcacgcgccacatgaagacgcacgggcagatcggcaaggaggtgtaccgctgcgacatctgccagatgcccttcagcgtctacagcaccctggagaaacacatgaaaaagtggcacggtgaacacttgctgactaatgatgtcaaaatcgagcaggctgagaggagctaagcgcatacgtgggggacactgcgtgcgtgcgtctgtacagcgtgaccatcgccaaccttcgccaacgggaccggtgaccggactggcctctgcatccccggggcccagggaggcggcagtccaacctaacctgtgtctgcgaagtcctatggaaacctgagggttgattaaggcagtaaaaaattaaaaaaaaaaaaaaa

SEQ ID NO: 46 - DNA, 1066 bp, Homo sapiens
ccggctcccagggccgccggtccccagggaaccccgcaggcttagccccacccggccggcgctgctcgctcccacgcccccgccgccgcttgtcgggagcgcacccagggagccagcggggcgcgggcgctgcaggggctgacatggacccaaatcctcgagcagccctggagcgccaacagctgcggctccgggagaggcagaagttctttgaggacattttacagccagagacagagtttgtcttccccctgtcccatctgcacctggagtcacaaagaccccccataggtagcatctcgtctatggaagtgaatgtggacacactggagcaagtggagtttattgatcttgcggatcaggatggagcagatgtgttcttgccttgtgaggagtcctcgccagctccccagatgtctggagtggatgaccatccagaggagctgagcctgctggtacccacgtctgacaggaccacatcccggacctcctccttgtcctctgactcctccaacctgcgcagtccaaatccaagtgatgggggaggagacactcccttggcacagtctgatgaggaggacggggatgacggaggggcagagcctggaccctgcagctagcagtgggcctcgtacagactgaccagcccggctgttctccatggagaggagacctaggcccagcagagcctggagaagacctgacactttccttacttcagcaccaaagggagggaaggatggtggatggtgtgcctgagagttagcctcccctgctttactgctaacgctatcctgctgccacgcccccacagtgcttttcttctgaggtaggacttccaagtgagactcgagaggtgaggtgggacaagacgcagctgctttcttagtcccctcctgcccccagatgatcctgttgtcttccacagagtctcctaagccagtgtctctgaggggatgttctgaggagttccactttccagttatcctgcctctataagttcttttgggaacaggatatggtataaataataaataataatataccactcaaaaaaaaaaaaaaa

SEQ ID NO: 47 - DNA, 1361 bp, Homo sapiens
ccctagttgaattaagaaactttaaaggttataaaagaaaaaaaaataaaatatttgttttctgctagatgcaaaaatgactaagcatgtatattttatcaacctcctgtattttttgaagttatgaactatataaaaatgttgacacttttttttgttttgttttgtttgctgggacagtgttaaataatttgcagtagttggttcatcctgcactgggactctcgggttgatgagcagcaggcggctgctgctgtcaggaggatgcacagttgcagtggtctccctgacgtgctttgttatacagagaaaccgtggactccatggaggaggggaggattggttcaacagctttattctctggaagcagtgacactcggtgattgtctgggaaaagggatcctttggtttccctgggtaccttgttccagacagtcttatttgccttcctagtatttagccccctctgacaattttttaaatgtgccttgtgggttggctagacactaaaatagaagtagaccagaggatactagagttgggacagttctctctttgttgggaggtgagcacgtttataggtagagagatttttggcttcgaagttctgtgccatctgcccagctggattgctatttcagcagtagaggttcctttttttttttttttcctccagtgacactgaccagaagtctgtccatccctctcccctttgcccttcccccaggctctcagccctccttaacactgacccaaaggtgcttgtaatgcaggtctcatctcctttgttagcgctcttcctatgactagaactggttaagcagtcaactgacgttaaccctgcttgcctaggccaaggttctctgttcctgccttctcaaattcccagcagagtgggctgtgttagggaagtgagtgctgggcacttgaggcccgtccctgcctgagcttctcctaccttctgctcattgtccccttacaagtatcacttttaggataactagccctggaaaaacggtatctgtttgaagtgaagggactggttcttcttgctttcagcccgtgtaaatatttaaataaaacagtattaacatttttgtgctgcttctcgttaggttgtagatctagccatgctctggccgcctctgccatcctggaggtagttttccttaacttccagaatagtgattttaaaactttaaaaaaaaaaagggggggggataacctatccttacatacaagcataacagattgtatctaactttatcacatatgatagagatgtatatgctgtaaagtgaggggaaggagctttctaataaaccagtgctttgtgcaaacttaaaaaaaaaaaaaaaaaa

SEQ ID NO: 48 - DNA, 3436 bp, Homo sapiens
cggacgcgtgggctctttcggatttgggcatttggagctggtgtcgcagatcctgagcaccatggctgaagaggaagtagggaacagccagaggcagagtgaagaaatcgaagcaatggcagccatttatggcgaggagtggtgtgtcattgatgaaaatgccaaaatattttgtattagagtcactgacttcatggatgaccccaaatggacactttgtttacaggtgatgttgccaagtgagtacccgggtacagcaccgccttcttatcagctgaacgctccctggctgaaagggcaagaacgcgcagacttatcgaacagccttgaggagatatatgtccacaacatgggtgaatctattctttaccagtgggtggagaaaataagagatgctctgatacagaaatctcagataaccgagccagacccagatgtcaagaagaaaactgaagaggttgaggttgagtccgaagaagaccctattctagagcacccgccggaaaatccagttaaaacattggacctcaaaatcagtgaagaaactcaaccagaaactgaagaattgcctccagttgctcatggcgttcctattactgaccgaaggagcacttttcaggctcatgtggcccctgtcgtctgtcctgagcaggtgaaactggttcttgccaagctgtacgagaataagaagattgccagcgccacccacaacatctatgcctacaggatattctgtgaggataaacagaccttcttgcaggactgtgaagatgatggagaaacagctgctgggggccgccttcttcacctcatggagattttgaatgtgaagaatgtcatggtggtggtgtcccgctggtatggagggattctgctgggacctgaccgtttcaaacacatcaacaactgtgccaggaacattctggtggagaagaacttcacgaacactcctgacgagtcaactaagaatttggggaaaaagaaggtgaagaaagacaagaagaagaatgatcattaagacgtgaacctgtgtgagaggttaacttcccagccactgtggacacgttcccagtggtcatgagtacattaagatagcacactgacagctcaagtcagccagttcatcatggttccaccagcattgtgtttcctcctctggttccaccatttgccagaactagagaatctagaacatattcacatgtgtttcacatttatttggaagaacagctgtgagcttacttaccatttcatataatactaggaagtgacacatgcacagtgttttcctgagctaaatgtcaatttcagtattgtcgcgtgcctttgttaggcagggaagacgtgtctccagaggaatagaaaggccacatgtcttgcgatagaaattgccatgctgttaagtgagcattgctttttcagctcccagttgctttcagagtgggggcgcatggaagacggaggtcttggggagtctttctcttgtgatttcacaagtgagtcacgcttccttccatagtattcatcatggagtgatagtttacgtgaaaacccgagtattcatctgtgatcatgtgccaaagtgatagtttacacatttgctagtccaagtgttactcctcagttgtttcaaattggtgattgtggaatttcaaaaagttactatttcttagttgttgccttttgttgtttgattcctgtagtgacacacagtgaccattcagtctcagtagtccttcccagtagtccttccgtcccatcccttacgtgtgttgaggtaacattccggtgtctctgaacgacctggaagcacttccctgtgctcttccctgtgctctggtcaactcacttgcagaacgagatagaggcagcaggacaccagaggagcaggctgcccactgcaccagtttgatatacatttgccatttgaactctggccagattttttttttttttagttctgtgattttttttttttttttttttgaggagagatagagggacaaatcttttgattagattaaaaaaaaaacctctttccctgcggaagtttttgtctaatctccttttttaaacacgtgtttaatttattttttacatgcatattacttgtaagaaataagacaaaaattccctttgtgtaggtaagctgtacctgttatctttcagaaataatgccaacaatgactaaattgatcaacggatcgcagactagtttgattcccctcccgaggaagaggcagccttgggagagtgctggagcacacatagacagcgtctgaggttgtgcaggttttttctcttagtttgtaagcttgaaagatccacatcctcacttggtattttaaataggaatccagtagccagctttacactagagtgggttctgagacagaagtcttattttcaacattatttaatgttgtaaattatcaagagttataggatgacctatatgtgtgtttggaacacttacctctgtttccatgaaatgctaagtatatcctatgtattaattgttacagagcttagacagtatttgctaaagataatgatataatcttgacatttcagacctgtgaaaagagattaaaacactaagaattttatgctgcagaaaaaaggagcataaaatattttcctagcacactggattagaatttctttcctggagttttgtctaaaatgtattagtgtttacagaagaaaagacaattttttatatctctgatctcattttccttcacttaaaaattacgtttccccattttacaagattgctaacctcattttgtgggttgagctccttaaccttcgctgcaggtgggaggggagaccccagttcctcacttctaactcctgaaattgcgtatttcacatgccattagtgcaaagtaatgatagcatagttttctctattgaagtagagaaaatcatatgtagagaattttctagttctcccaattataactttgtaaagccaggcgtctgctgtggccttctctatattttattagttgacatgttctccacatgccagtatctatgagcaaatggcatccacgtagacgggcagagcagaagctgctagtgaggtgtctgtcagagacaggctgttggaaggaagcactgatacaggacagataaagcagatactgtttacagggcaggtgagtgatactgtaatcacttcagtgaattgtaatcacatccagtccactaaagagatgaccataaatggtgtatacagtttttattttttattatgcatgattttgtatatatggctatttttctttccgtaaaaatggtattaaaccgtatatactgttttgtatcctacatatttcatatagaagtatattgttaacattttcccatatcaataaatattcttctatggcttaaaaaaaaaaaaaaaaaaaaaaaaaaaaaa

SEQ ID NO: 49 - protein, 730 aa, Homo sapiens
MAAGVEAAAEVAATEIKMEEESGAPGVPSGNGAPGPKGEGERPAQNEKRKEKNIKRGGNRFEPYANPTKRYRAFITNIPFDVKWQSLKDLVKEKVGEVTYVELLMDAEGKSRGCAVVEFKMEESMKKAAEVLNKHSLSGRPLKVKEDPDGEHARRAMQKVMATTGGMGMGPGGPGMITIPPSILNNPNIPNEIIHALQAGRLGSTVFVANLDYKVGWKKLKEVFSMAGVVVRADILEDKDGKSRGIGTVTFEQSIEAVQAISMFNGQLLFDRPMHVKMDERALPKGDFFPPERPQQLPHGLGGIGMGLGPGGQPIDANHLNKGIGMGNIGPAGMGMEGIGFGINKMGGMEGPFGGGMENMGRFGSGMNMGRINEILSNALKRGEIIAKQGGGGGGGSVPGIERMGPGIDRLGGAGMERMGAGLGHGMDRVGSEIERMGLVMDRMGSVERMGSGIERMGPLGLDHMASSIERMGQTMERIGSGVERMGAGMGFGLERMAAPIDRVGQTIERMGSGVERMGPAIERMGLSMERMVPAGMGAGLERMGPVMDRMATGLERMGANNLERMGLERMGANSLERMGLERMGANSLERMGPAMGPALGAGIERMGLAMGGGGGASFDRAIEMERGNFGGSFAGSFGGAGGHAPGVARKACQIFVRNLPFDFTWKMLKDKFNECGHVLYADIKMENGKSKGCGVVKFESPEVAERACRMMNGMKLSGREIDVRIDRNA

SEQ ID NO: 50 - protein, 118 aa, Homo sapiens
MRRAGLGEGVPPGNYGNYGYANSGYSACEEENERLTESLRSKVTAIKSLSIEIGHEVKTQNKLLAEMDSQFDSTTGFLGKTMGKLKILSRGSQTKLLCYMMLFSLFVFFIIYWIIKLR

SEQ ID NO: 51 - protein, 205 aa, Homo sapiens
MSSMNPEYDYLFKLLLIGDSGVGKSCLLLRFADDTYTESYISTIGVDFKIRTIELDGKTIKLQIWDTAGQERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

SEQ ID NO: 52 - protein, 237 aa, Homo sapiens
MNIFDRKINFDALLKFSHITPSTQQHLKKVYASFALCMFVAAAGAYVHMVTHFIQAGLLSALGSLILMIWLMATPHSHETEQKRLGLLAGFAFLTGVGLGPALEFCIAVNPSILPTAFMGTAMIFTCFTLSALYARRRSYLFLGGILMSALSLLLLSSLGNVFFGSIWLFQANLYVGLVVMCGFVLFDTQLIIEKAEHGDQDYIWHCIDLFLDFITVFRKLMMILAMNEKDKKKEKK

SEQ ID NO: 53 - protein, 299 aa, Homo sapiens
MEEFDSEDFSTSEEDEDYVPSGGEYSEDDVNELVKEDEVDGEEQTQKTQGKKRKAQSIPARKRRQGGLSLEEEEEEDANSESEGSSSEEEDDAAEQEKGIGSEDARKKKEDELWASFLNDVGPKSKVPPSTQVKKGEETEETSSSKLLVKAEELEKPKETEKVKITKVFDFAGEEVRVTKEVDATSKEAKSFFKQNEKEKPQANVPSALPSLPAGSGLKRSSGMSSLLGKIGAKKQKMSTLEKSKLDWESFKEEEGIGEELAIHNRGKEGYIERKAFLDRVDHRQFEIERDLRLSKMKP

SEQ ID NO: 54 - protein, 434 aa, Homo sapiens
MVSSCELELQEQSLRTASDQESGDEELNRLKEENEKLRSLTFSLAEKDILEQSLDEARGSRQELVERIHSLRERAVAAERQREQYWEEKEQTLLQFQKSKMACQLYREKVNALQAQVCELQKERDQAYSARDSAQREISQSLVEKDSLRRQVFELTDQVCELRTQLRQLQAEPPGVLKQEARTREPCPREKQRLVRMHAICPRDDSDCSLVSSTESQLLSDLSATSSRELVDSFRSSSPAPPSQQSLYKRVAEDFGEEPWSFSSCLEIPEGDPGALPGAKAGDPHLDYELLDTADLPQLESSLQPVSPGRLDVSESGVLMRRRPARRILSQVTMLAFQGDALLEQISVIGGNLTGIFIHRVTPGSAADQMALRPGTQIVMVSRARPLLSPGLLMGTVAAGGVTQADFTSPRRCRSTLGWASALSWADVKRSAHL

SEQ ID NO: 55 - protein, 121 aa, Homo sapiens
MTCHPRRAPGSQGLVDERVVARPSGEPSVKEEEDKASAAEGEGVQQLREALKILAERVLILEHMIGVHDPLASPEGGSGQDAALRANLKMKRGGPRPDGILAALLGPDPAQKSADQAGDRK

SEQ ID NO: 56 - protein, 490 aa, Homo sapiens
MTSHSTSAQCSASDSACRISSEQISQQVRPKLQLLKILHAAGAQGEVFTMKEVMHYLGQYIMVKQLYDQQEQHMVYCGGDLLGDLLGCQSFSVKDPSPLYDMLRKNLVTSASNNTDAAQTLALAQDHTMDFPSQDRLKHGATEYSNPRKRTEEEDTHTLPTSRHKCRDSRADEDLIEHLSQDETSRLDLDFEEWDVAGLPWWFLGNLRNNCIPKSNGSTDLQTNQDIGTAIVSDTTDDLWFLNETVSEQLGVGIKVEAANSEQTSEVGKTSNKKTVEVGKDDDLEDSRSLSDDTDVELTSEDEWQCTECKKFNSPSKRYCFRCWALRKDWYSDCSKLTHSLSTSNITAIPEKKDNEGIDVPDCRRTISAPVVRPKDGYLKEEKPRFDPCNSVGFLDLAHSSESQEIISSAREQTDIFSEQKAETESMEDFQNVLKPCSLCEKRPRDGNIIHGKTSHLTTCFHCARRLKKSGASCPVCKKEIQLVIKVFIA

SEQ ID NO: 57 - protein, 269 aa, Homo sapiens
MASANTRRVGDGAGGAFQPYLDSLRQELQQRDPTLLSVAVALLAVLLTLVFWKFIWSRKSSQRAVLFVGLCDSGKTLLFVRLLTGQYRDTQTSITDSSAIYKVNNNRGNSLTLIDLPGHESLRFQLLDRFKSSARAVVFVVDSAAFQREVKDVAEFLYQVLIDSMALKNSPSLLIACNKQDIAMAKSAKLIQQQLEKELNTLRVTRSAAPSTLDSSSTAPAQLGKKGKEFEFSQLPLKVEFLECSAKGGRGDTGSADIQDLEKWLAKIA

SEQ ID NO: 58 - protein, 363 aa, Homo sapiens
MKDNFSFAATSRNITSSRPFDNLNATGTNESAFNCSHKPSDKHLEAIPVLYYMIFVIGFAVNIVVVSLFCCQKGPKKVSSIYIFNLALADLLLLATLPLWATYYSYRYDWLFGPVMCKVFGSFLTLNMFASIFFITCMSVDRYQSVIYPFLSQRRNPWQASYVVPLVWCMACLSSLPTFYFRDVRTIEYLGVNACIMAFPPEKYAQWSAGIALMKNILGFIIPLIFIATCYFGIRKHLLKTNSYGKNRITRDQVLKMAAAVVLAFIICWLPFHVLTFLDALTWMGIINSCEVIAVIDLALPFAILLGFTNSCVNPFLYCFVGNRFQQKLRSVFRVPITWLQGKRETMSCRKGSSLREMDTFVS

SEQ ID NO: 59 - protein, 424 aa, Homo sapiens
MEAVYLVVNGVGLVLDLLTLMLDLNFLLVSSLLATLAWLLAFIYNLPHTVLTSLLHLGRGFLLSLLALVEAVVRFTFGGLQALGTVLYSCYSGLESLKLLGHLASHGALRSREFLNRGILNMVSNGHALLRQACDICAIAMSLVAYVINSLVNICLIGTQNFFSLVLALWDAVTGPLWRMTDVVAAFLAHISSSAVAMAILLWTPCQLALELLASAARLLASCVVFHLTGLVLLACVLAVILIVLHPEQTLRLATQALSQLHARPSYHRLWEDIVRLTRLPLGLEAWRRVWSRSLQLASWPNRGGAPGAPQGGPRRVFSARIQPQDTPPEAEEEVIRAAPARGREQLNEDEPAAGQDPWKLLKEQEERKKCVICQDQSKTVLLLPCRHLCLCQACTEILMRHPVYHRNCPLCRRSILQTLNVYL

SEQ ID NO: 60 - protein, 301 aa, Homo sapiens
MGAQLSTLGHMVLFPVWFLYSLLMKLFQRSTPAITLESPDIKYPLRLIDREIISHDTRRFRFALPSPQHILGLPVGQHIYLSARIDGNLVVRPYTPISSDDDKGFVDLVIKVYFKDTHPKFPAGGKMSQYLESMQIGDTIEFRGPSGLLVYQGKGKFAIRPDKKSNPIIRTVKSVGMIAGGTGITPMLQVIRAIMKDPDDHTVCHLLFANQTEKDILLRPELEELRNKHSARFKLWYTLDRAPEAWDYGQGFVNEEMIRDHLPPPEEEPLVLMCGPPPMIQYACLPNLDHVGHPTERCFVF

SEQ ID NO: 61 - protein, 222 aa, Homo sapiens
MKSHYIVLALASLTFLLCLPVSQSCNKALCASDVSKCLIQELCQCRPGEGNCPCCKECMLCLGALWDECCDCVGRCNPRNYSDTPPTSKSTVEELHEPIPSLFRALTEGDTQLNWNIVSFPVAEELSHHENLVSFLETVNQLHHQNVSVPSNNVHAPFPSDKERMCTVVYFDDCMSIHQCKISCESMGASKYRWFHNACCECIGPECIDYGSKTVKCMNCMF

SEQ ID NO: 62 - protein, 390 aa, Homo sapiens
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 63 - protein, 129 aa, Homo sapiens
MRTLDLIDEAYGFDFYILKTPKEDLGSKFGMDLKRGMLLRLARQDPHLHPENPERRAAIYDKYRSFVIPEAEAEWVGLTLEEALEKQRLLEEKDPVPLFKVYVEELVQRLQEQVLSRPAVVQKRAGDHA

SEQ ID NO: 64 - protein, 520 aa, Homo sapiens
MNSSTSAANGNDNKKFKGDRPPCSPSRVLHLRKIPCDVTEAEVISLGLPFGKVTNLLMLKGKSQAFLEMASEEAAVTMINYYTPVTPHLRSQPVYIQYSNHRELKTDNLPNQARAQAALQAVSAVQSGNLSLPGATANEGTLLPGQSPVLRIIIENLFYPVTLEVLHQIFSKFGTVLKIITFTKNNQFQALLQYADPVNAQYAKMALDGQNIYNACCTLRIDFSKLTSLNVKYNNDKSRDFTRLDLPTGDGQPSLEPPMAAAFGAPGIMSSPYAGAAGFAPAIAFPQAAGLSVPAVPGALGPLTLTSSAVSGRMAIPGASGMPGNSVLLVTNLNPDFITPHGLFILFGVYGDVHRVKIMFNKKENALVQMADASQAQLAMNHLSGQRLYGKVLRATLSKHQAVQLPREGQEDQGLTKDFSNSPLHRFKKPGSKNFQNIFPPSATLHLSNIPPSVTMDDLKNLFTEAGCSVKAFKFFQKDRKMALIQLGSVEEAIQALIELHNHDLGENHHLRVSFSKSTI

SEQ ID NO: 65 - protein, 309 aa, Homo sapiens
MEKYLTPQLPPVPIISEHKKYRRDSASVVDQFFTDTEGIPYSINMNVFLPDITHLRTGLYKSQRPCVTQIKTEPVTIFSHQSESTAPPPPPAPTQALPEFTSIFSSHQTTAPPQEVNNIFIKQELPIPDLHLSVPSQQGHLYQLLNTPDLDMPSSTNQTAVMDTLNVSMAGLNPHPSAVPQTSMKQFQGMPPCTYTMPSQFLPQQATYFPPSPPSSEPGSPDRQAEMLQNLTPPPSYAATIASKLAIHNPNLPATLPVNSPTLPPVRYNRRSNPDLEKRRIHFCDYNGCTKVYTKSSHLKAHLRTHTGR

SEQ ID NO: 66 - protein, 727 aa, Homo sapiens
MLRIPIKRALIGLSNSPKGYVRTTGTAASNLIEVFVDGQSVMVEPGTTVLQACEKVGMQIPRFCYHERLSVAGNCRMCLVEIEKAPKVVAACAMPVMKGWNILTNSEKSKKAREGVMEFLLANHPLDCPICDQGGECDLQDQSMMFGSDRSRFLEGKRAVEDKNIGPLVKTIMTRCIQCTRCIRFASEIAGVDDLGTTGRGNDMQVGTYIEKMFMSELSGNVIDICPVGALTSKPYAFTARPWETRKTESIDVMDAVGSNIVVSTRTGEVMRILPRMHEDINEEWISDKTRFAYDGLKRQRLTEPMVRNEKGLLTYTSWEDALSRVAGMLQNFEGNAVAAIAGGLVDAEALVALKDLLNKVDSDNLCTEEIFPTEGAGTDLRSNYLLNTTIAGVEEADVVLLVGTNPRFEAPLFNARIRKSWLHNDLKVALIGSPVDLTYRYDHLGDSPKILQDIASGRHSFCEVLKDAKKPMVVLGSSALQRDDGAAILAAVSNMVQKIRVTTGVAAEWKVMNILHRIASQVAALDLGYKPGVEAIRKNPPKMLFLLGADGGCITRQDLPKDCFIVYQGHHGDVGAPMADVILPGAAYTEKSATYVNTEGRAQQTKVAVTPPGLAREDWKIIRALSEIAGITLPYDTLDQVRNRLEEVSPNLVRYDDIEETNYFQQASELAKLVNQEVLADPLVPPQLTIKDFYMTDSISRASQTMAKCVKAVTEGAQAVEEPSIC

SEQ ID NO: 67 - protein, 111 aa, Homo sapiens
MSKRKAPQETLNGGITDMLVELANFEKNVSQAIHKYNAYRKAASVIAKYPHKIKSGAEAKKLPGVGTKIAEKIDEFLATGKLRKLEKIRQDDTSSSINFLTRVTGIGSQEK

SEQ ID NO: 68 - protein, 317 aa, Homo sapiens
MAKNPPENCEGCHILNAEALKSKKICKSLKICGLVFGILALTLIVLFWGSKHFWPEVSKKTYDMEHTFYSNGEKKKIYMEIDPITRTEIFRSGNGTDETLEVHDFKNGYTGIYFVGLQKCFIKTQIKVIPEFSEPEEEIDENEEITTTFFEQSVIWVPAEKPIENRDFLKNSKILEICDNVTMYWINPTLIAVSELQDFEEDGEDLHFPTSEKKGIDQNEQWVVPQVKVEKTRHTRQASEEDLPINDYTENGIEFDPMLDERGYCCIYCRRGNRYCRRVCEPLLGYYPYPYCYQGGRVICRVIMPCNWWVARMLGRV

SEQ ID NO: 69 - protein, 504 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQAFSPSDPDCVDRLLQCTRQAVPLFSKNVHSTRFVTYFCEQVLPNLSTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQKVEIGQKRTSEDTSSGSPPKKSPGGPKRDARQIYNPPSGKYSSNLSNFNYERSLQGK

SEQ ID NO: 70 - protein, 127 aa, Homo sapiens
MRPLDIDEVEAPEEVEVLEPEEDFEQFLLPVINEMREDIASLIREHGRAYLRTRSKLWEMDNMLIQIKTQVEASEESALNHVQHPSGEADERVSELCEKAEEKAKEIAKMAEMLVELVWRIERSESS

SEQ ID NO: 71 - protein, 103 aa, Homo sapiens
MAHYKAADSKREQFRRYLEKSGVLDTLTKVLVALYEEPEKPNSALDFLKHHLGAATPENPEIELLRLELAEMKEKYEAIVEENKKLKAKLAQYEPPQEEKRAE

SEQ ID NO: 72 - protein, 31 aa, Homo sapiens
MPPHSGQSRGRKPGSCLLLLLLLLLLLLLLL

SEQ ID NO: 73 - protein, 226 aa, Homo sapiens
MALSDVDVKKQIKHMMAFIEQEANEKAEEIDAKAEEEFNIEKGRLVQTQRLKIMEYYEKKEKQIEQQKKILMSTMRNQARLKVLRARNDLISDLLSEAKLRLSRIVEDPEVYQGLLDKLVLQGLLRLLEPVMIVRCRPQDLLLVEAAVQKAIPEYMTISQKHVEVQIDKEAYLAVNAAGGVEVYSGNQRIKVSNTLESRLDLSAKQKMPEIRMALFGANTNRKFFI

SEQ ID NO: 74 - protein, 165 aa, Homo sapiens [sequence omitted]

SEQ ID NO: 75 - protein, 359 aa, Homo sapiens
MIAAQAKLVYQLNKYYTERCQARKAAIAKTIREVCKVVSDVLKEVEVQEPRFISSLSEIDARYEGLEVISPTEFEVVLYLNQMGVFNFVDDGSLPGCAVLKLSDGRKRSMSLWVEFITASGYLSARKIRSRFQTLVAQAVDKCSYRDVVKMIADTSEVKLRIRERYVVQITPAFKCTGIWPRSAAQWPMPHIPWPGPNRVAEVKAEGFNLLSKECYSLTGKQSSAESDAWVLQFGEAENRLLMGGCRNKCLSVLKTLRDRHLELPGQPLNNYHMKTLLLYECEKHPRETDWDESCLGDRLNGILLQLISCLQCRRCPHYFLPNLDLFQGKPHSALESAAKQTWRLAREILTNPKSLDKL

SEQ ID NO: 76 - protein, 640 aa, Homo sapiens
MAAMYLPGLRLSRHGLRPWCWSPCRSIQTLRVLSGDMSQLPAGVRDFVARSAHLCQPEGIHICDGTEAENTAILALLEEQGLIRKLPKYKNCWLARTDPKDVARVESKTVIVTPSQRDTVPLLAGGARGQLGNWMSPDEFQRAVDERFPGCMQGRIMYVLPFSMGPVGSPLSRIGVQLTDSAYVVASMRIMTRLGTPVLQALGDGDFIKCLHSVGQPLTGHGDPVGQWPCNPEKTLIGHVPDQREIVSFGSGYGGNSLLGKKCFALRIASRLARDEGWLAEHMLILGITNPAGKKRYVAAAFPSACGKTNLAMMRPALPGWKVECVGDDIAWMRFDSEGQLRAINPENGFFGVAPGTSAATNPNAMATIQSNTLFTNVAETSDGGVYWEGIDQPLPPGVTITSWLGKPWKPGDKEPCAHPNSRFCVPARQCPIMDPAWEAPEGVPIDAIIFGGRRPKGVPLVYEAFNWRHGVFVGSAMRSESTAAAEHKGKTIMHDPFAMRPFFGYNFGRYLEHWLSMEGQKGARLPRIFHVNWFRRDEAGCFLWPGFGENARVLDWICRRLEGEDSAQETPIGLVPKEGALDLSGLSAVDTSQLFSIPKDFWEQEVRDIRGYLTEQVNQDLPKEVLAELEALEGRVQKM

SEQ ID NO: 77 - protein, 108 aa, Homo sapiens
MVLQRIFRLSSVLRSAVSVHLKRNIGVTAVAFNKELDPVQKLFVDKIREYKSKRQASGGPVDIGPEYQQDLDRELYKLKQMYGKGEMDTFPTFKFDDPKFEVIDKPQS

SEQ ID NO: 78 - protein, 317 aa, Homo sapiens
MHGRLKVKTSEEQAEAKRLEREQKLKLYQSATQAVFQKREAGELDESVLELTSQILGANPDFATLWNCRREVLQQLETQKSPEELAALVKAELGFLESCLRVNPKSYGTWHHRCWLLSRLPEPNWARELELCARFLEADERNFHCWDYRRFVAAQAAVAPAEELAFTDSLITRNFSNYSSWHYRSCLLPQLHPQPDSGPQGRLPENVLLRELELVQNAFFTDPNDQSAWFYHRWLLGRAEPHDVLCCLHVSREEACLSVCFSRPLIVGSKMGTLLLTVDEAPLSVEWRTPDGRNRPSHVWVSRDWWGRIRVGQSEKQ

SEQ ID NO: 79 - protein, 127 aa, Homo sapiens
MYTYVCIYTYVCIHMCVYIRMYVYICVYIYVCMYTYVCIYTYVCIHICVYIRVYVYIYVYIYVCMYTYMCIYTCVCIHICVYIRVYVYIYVYIYVCMYTYVCIYTCVYIYTYIRIYVYIYTQLNQWD

SEQ ID NO: 80 - protein, 493 aa, Homo sapiens
MLETYRNLNAVGYNWEDSNIEEHCESSRRHGRHERNHTGEKPYEGIQYGEAFVHHSSLQMRKIIHTGEKRYKCNQCDKAYSRHSILQIHKRTHSGEKPYECNQCGKAFTQHSHLKIHMVTHTGEKPYKCDQCGKAFAFHSTLQVHKRTHTGEKPYECNQCSKAFAHHCHLRVHKRIHTGEKPYKCDQCGKAFVGQNDLKRHERVHTGEKPYKCNECGKAFVCNASLRTHKTTHTGVKPYECKQCTKSFASHGQLQKHERIHTGEKPYKCDQCGKAFASHDKFQKHERIHTGEKPYKCKQCTKSFASHDKLQKHERIHTGEKPYECKQCTKSFASHNKLQKHERIHTGEKPYKCDQCNKAFVYESYLQVHKKTHTGEKPYKCNECGKAFARHSHLKVHKITHTGEKPYKCNQCGKALAYHSTLQVHQRTHTGEKPYECEQCGKAFANQSYFQVHKRIHTGEKPYKCDQCGKAFVGSSDLKRHERVHTGRETLQM

SEQ ID NO: 81 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 82 - protein, 313 aa, Homo sapiens
MDTSTNLDIGAQLIVEECPSTYSLTGMPDIKIEHPLDPNSEEGSAQGVAMGMKFILPNRFDMNVCSRFVKSLNEEDSKNIQDQVNSDLEVASVLFKAECNIHTSPSPGIQVRHVYTPSTTKHFSPIKQSTTLTNKHRGNEVSTTPLLANSLSVHQLAAQGEMLYLATRIEQENVINHTDEEGFTPLMWAAAHGQIAVVEFLLQNGADPQLLGKGRESALSLACSKGYTDIVKMLLDCGVDVNEYDWNGGTPLLYAVHGNHVKCVKMLLESGADPTIETDSGYNSMDLAVALGYRSVQQVIESHLLKLLQNIKE

SEQ ID NO: 83 - protein, 396 aa, Homo sapiens
EEGRAVHGAAPQDLSRNLEFSELPDHCYRMNSSPAGTPSPQPSRANGNINLGPSANPNAQPTDFDFLKVIGKGNYGKVLLAKRKSDGAFYAVKVLQKKSILKKKEQSHIMAERSVLLKNVRHPFLVGLRYSFQTPEKLYFVLDYVNGGELFFHLQRERRFLEPRARFYAAEVASAIGYLHSLNIIYRDLKPENILLDCQGHVVLTDFGLCKEGVEPEDTTSTFCGTPEYLAPEVLRKEPYDRAVDWWCLGAVLYEMLHGLPPFYSQDVSQMYENILHQPLQIPGGRTVAACDLLQSLLHKDQRQRLGSKADFLEIKNHVFFSPINWDDLYHKRLTPPFNPNVTGPADLKHFDPEFTQEAVSKSIGCTPDTVASSSGASSAFLGFSYAPEDDDILDC

SEQ ID NO: 84 - protein, 494 aa, Homo sapiens
MTLLHYTCKSGAHGIGDVETAVKFATQLIDLGADISLRSRWTNMNALHYAAYFDVPELIRVILKTSKPKDVDATCSDFNFGTALHIAAYNLCAGAVKCLLEQGANPAFRNDKGQIPADVVPDPVDMPLEMADAAATAKEIKQMLLDAVPLSCNISKAMLPNYDHVTGKAMLTSLGLKLGDRVVIAGQKVGTLRFCGTTEFASGQWAGIELDEPEGKNNGSVGKVQYFKCAPKYGIFAPLSKISKAKGRRKNITHTPSTKAAVPLIRSQKIDVAHVTSKVNTGLMTSKKDSASESTLSLPPGEELKTVTEKDVALLGSVSSCSSTSSLEHRQSYPKKQNAISSNKKTMSKSPSLSSRASAGLNSSATSTANNSRCEGELRLGERVLVVGQRLGTIRFFGTTNFAPVLGYWYGIELEKPHGKNDGSVGGVQYFSCSPRYGIFAPPSRVQRVTDSLDTLSEISSNKQNHSYPGFRRSFSTTSASSQKEINRRNAFSK

SEQ ID NO: 85 - protein, 301 aa, Homo sapiens
MLALRVARGSWGALRGAAWAPGTRPSKRRACWALLPPVPCCLGCLAERWRLRPAALGLRLPGIGQRNHCSGAGKAAPRPAAGAGAAAEAPGGQWGPASTPSLYENPWTIPNMLSMTRIGLAPVLGYLIIEEDFNIALGVFALAGLTDLLDGFIARNWANQRSALGSALDPLADKILISILYVSLTYADLIPVPLTYMIISRDVMLIAAVFYVRYRTLPTPRTLAKYFNPCYATARLKPTFISKVNTAVQLILVAASLAAPVFNYADSIYLQILWCFTAFTTAASAYSYYHYGRKTVQVIKD

SEQ ID NO: 86 - protein, 427 aa, Homo sapiens
MSDNQSWNSSGSEEDPETESGPPVERCGVLSKWTNYIHGWQDRWVVLKNNTLSYYKSEDETEYGCRGSICLSKAVITPHDFDECRFDISVNDSVWYLRAQDPEHRQQWVDAIEQHKTESGYGSESSLRRHGSMVSLVSGASGYSATSTSSFKKGHSLREKLAEMETFRDILCRQVDTLQKYFDVCADAVSKDELQRDKVVEDDEDDFPTTRSDGDFLHNTNGNKEKLFPHVTPKGINGIDFKGEAITFKATTAGILATLSHCIELMVKREESWQKRHDREVEKRRRVEEAYKNVMEELKKKPRFGGPDYEEGPNSLINEEEFFDAVEAALDRQDKIEEQSQSEKVRLHWPTSLPSGDTFSSVGTHRFVQKPYSRSSSMSSIDLVSASDDVHRFSSQVEEMVQNHMNYSLQDVGGALYTPKVLVSFLY

SEQ ID NO: 87 - protein, 363 aa, Homo sapiens
MDAARRGDTQPVMWTTGWLLLLPLLLCEGAQALECYSCVQKADDGCSPHRMKTVKCGPGVDVCTEAVGAVETIHGQFSVAVRGCGSGIPGKNDRGLDLHGLLAFFQLQQCSEDRCNAKLNLTLRGLNPAGNESAYEPNGAECYSCVGLSREKCQGSMPPVVNCYNASGRVYKGCFDGNVTLTAANVTVSLPVRGCVQDETCTRDGVTGPGFTLSGSCCQGPRCNADLRNKTYFSPRIPPLVLLPPPTTAAPSTRAQNSSSTTSTAAPTTTTSIIKPTTAQASHTSPHEMDLEVIQEEGASLSGGAAGHGGTAGHGGAAGHQDRSNMEKYPGKGGAQIPAKGGSGTLGSWLSAVLLTVVAGAML

SEQ ID NO: 88 - protein, 110 aa, Homo sapiens
MLEGEQEEERKRELEKKQRKEKEKILLQKREIESKLFGDPDEFPLAHLLEPFRQYYLQAEHSLPALIQIRHDWDQYLVPSDHPKGNFVPQGWVLPPLPSNDIWATAVKLH

SEQ ID NO: 89 - protein, 469 aa, Homo sapiens
MKDCPMPRNAARISEKRKEYMDACGEANNQNFQQRYHAEEVEERFGRFKPGVISEELQDALGVTDKSLPPFIYRMRQLGYPPGWLKEAELENSGLALYDGKDGTDGETEVGEIQQNKSVTYDLSKLVNYPGFNISTPRGIPDEWRIFGSIPMQACQQKDVFANYLTSNFQAPGVKSGNKRSSSHSSPGSPKKQKNESNSAGSPADMELDSDMEVPHGSQSSESFQFQPPLPPDTPPLPRGTPPPVFTPPLPKGTPPLTPSDSPQTRTASGAVDEDALTLEELEEQQRRIWAALEQAESVNSDSDVPVDTPLTGNSVASSPCPNELDLPVPEGKTSEKQTLDEPEVPEIFTKKSEAGHASSPDSEVTSLCQKEKAELAPVNTEGALLDNGSVVPNCDISNGGSQKLFPADTSPSTATKIHSPIPDMSKFATGITPFEFENMAESTGMYLRIRSLLKNSPRNQQKNKKASE

SEQ ID NO: 90 - protein, 648 aa, Homo sapiens [sequence omitted]

SEQ ID NO: 91 - protein, 812 aa, Homo sapiens
MSRRKQGNPQHLSQRELITPEADHVEATILEEDEGLEIEEPSSLGLMVGGPDPDLLTCGQCQMNFPLGDILVFIEHKKKQCGGLGPCYDKVLDKSSPPPSSRSELRRVSEPVEIGIQVTPDEDDHLLSPTKGICPKQENIAGKDEPSSYICTTCKQPFNSAWFLLQHAQNTHGFRIYLEPGPASTSLTPRLTIPPPLGPETVAQSPLMNFLGDSNPFNLLRMTGPILRDHPGFGEGRLPGTPPLFSPPPRHHLDPHRLSAEEMGLVAQHPSAFDRVMRLNPMAIDSPAMDFSRRLRELAGNSSTPPPVSPGRGNPMHRLLNPFQPSPKSPFLSTPPLPPMPAGTPPPQPPAKSKSCEFCGKTFKFQSNLIVHRRSHTGEKPYKCQLCDHACSQASKLKRHMKTHMHKAGSLAGRSDDGLSAASSPEPGTSELPGDLKAADGDFRHHESDPSLGPEPEDDEDEEEEEEELLLENESRPESSFSMDSELGRGRENGGGVPPGVAGAGAAAAALADEKALALGKVMEDAGLGALPQYGEKRGAFLKRAGDTGDAGAVGCGDAGAPGAVNGRGGAFAPGAEPFPALFPRKPAPLPSPGLGGPALHAAKRIKVEKDLELPPAALIPSENVYSQWLVGYAASRHFMKDPFLGFTDARQSPFATSSEHSSENGSLRFSTPPGDLLDGGLSGRSGTASGGSTPHLGGPGPGRPSSKEGRRSDTCEYCGKVFKNCSNLTVHRRSHTGERPYKCELCNYACAQSSKLTRHMKTHGQIGKEVYRCDICQMPFSVYSTLEKHMKKWHGEHLLTNDVKIEQAERS

SEQ ID NO: 92 - protein, 158 aa, Homo sapiens
MDPNPRAALERQQLRLRERQKFFEDILQPETEFVFPLSHLHLESQRPPIGSISSMEVNVDTLEQVEFIDLADQDGADVFLPCEESSPAPQMSGVDDHPEELSLLVPTSDRTTSRTSSLSSDSSNLRSPNPSDGGGDTPLAQSDEEDGDDGGAEPGPCS

SEQ ID NO: 93 - protein, 65 aa, Homo sapiens
MCLVGWLDTKIEVDQRILELGQFSLCWEVSTFIGREIFGFEVLCHLPSWIAISAVEVPFFFFFLQ

SEQ ID NO: 94 - protein, 318 aa, Homo sapiens
MAEEEVGNSQRQSEEIEAMAAIYGEEWCVIDENAKIFCIRVTDFMDDPKWTLCLQVMLPSEYPGTAPPSYQLNAPWLKGQERADLSNSLEEIYVHNMGESILYQWVEKIRDALIQKSQITEPDPDVKKKTEEVEVESEEDPILEHPPENPVKTLDLKISEETQPETEELPPVAHGVPITDRRSTFQAHVAPVVCPEQVKLVLAKLYENKKIASATHNIYAYRIFCEDKQTFLQDCEDDGETAAGGRLLHLMEILNVKNVMVVVSRWYGGILLGPDRFKHINNCARNILVEKNFTNTPDESTKNLGKKKVKKDKKKNDH